(19) United States
(12) Patent Application Publication
(10) Pub. No.: US 2015/0294064 A1
(43) Pub. Date: Oct. 15, 2015

(54) IDENTIFICATION, MONITORING AND TREATMENT OF INFECTIOUS DISEASE AND CHARACTERIZATION OF INFLAMMATORY CONDITIONS RELATED TO INFECTIOUS DISEASE USING GENE EXPRESSION PROFILES

(71) Applicant: LIFE TECHNOLOGIES CORPORATION, Carlsbad, CA (US)

(72) Inventors: Michael BEVILACQUA, Boulder, CO (US); Danute BANKAITIS-DAVIS, Longmont, CO (US); John CHERONIS, Conifer, CO (US); Victor TRYON, Loveland, CO (US)

(21) Appl. No.: 14/685,373

(22) Filed: Apr. 13, 2015

Related U.S. Application Data
(63) Continuation of application No. 13/110,714, filed on May 18, 2011.
Application No. 13/110,714 is a continuation of application No. 10/742,458, filed on Dec. 19, 2003, now abandoned.
Application No. 10/742,458 is a continuation-in-part of application No. 10/291,225, filed on Nov. 8, 2002, now Pat. No. 6,960,439.
Application No. 10/291,225 is a continuation-in-part of application No. 09/821,850, filed on Mar. 29, 2001, now Pat. No. 6,692,916.
Application No. 09/821,850 is a continuation-in-part of application No. 09/605,581, filed on Jun. 28, 2000, now abandoned.
(60) Provisional application No. 60/435,257, filed on Dec. 19, 2002.
(60) Provisional application No. 60/141,542, filed on Jun. 28, 1999.
(60) Provisional application No. 60/195,522, filed on Apr. 7, 2000.

(51) Int. Cl. G06F 19/18; A61B 5/145; A61B 5/0205; G06F 19/00

(52) U.S. Cl. CPC G06F 19/18; G06F 19/345; A61B 5/145; A61B 5/02055; A61B 5/01

(57) ABSTRACT

A method is provided in various embodiments for determining a profile data set for a subject with infectious disease or inflammatory conditions related to infectious disease based on a sample from the subject, wherein the sample provides a source of RNAs. The method includes using amplification for measuring the amount of RNA corresponding to at least 2 constituents from Table 1. The profile data set comprises the measure of each constituent, and amplification is performed under measurement conditions that are substantially repeatable.

### CROSS-REFERENCE TO RELATED APPLICATIONS

This application is a continuation of U.S. application Ser. No. 13/110,714 filed May 18, 2011, which is a continuation of U.S. application Ser. No. 10/742,458 filed Dec. 19, 2003, now abandoned, which claims priority benefit to U.S. Provisional Application No. 60/435,257 filed Dec. 19, 2002 and is a continuation-in-part of U.S. application Ser. No. 10/291,225 filed Nov. 8, 2002 (now U.S. Pat. No. 6,960,439), which is a continuation-in-part of U.S. application Ser. No. 09/821,850 filed Mar. 29, 2001 (now U.S. Pat. No. 6,692,916), which is continuation-in-pan of U.S. application Ser. No. 09/605,581 filed Jun. 28, 2000, now abandoned which claims priority benefit to U.S. application No. 60/141,542 filed Jun. 28, 1999 and 60/195,522 filed Apr. 7, 2000, which disclosures are herein incorporated by reference in their entirety.

### TECHNICAL FIELD AND BACKGROUND ART

The present invention relates to use of gene expression data, and in particular to use of gene expression data in identification, monitoring and treatment of infectious disease and in characterization and evaluation of inflammatory conditions of a subject induced or related to infectious disease.

The prior art has utilized gene expression data to determine the presence or absence of particular markers as diagnostic of a particular condition, and in some circumstances have described the cumulative addition of scores for over expression of particular disease markers to achieve increased accuracy or sensitivity of diagnosis. Information on any condition of a particular patient and a patient's response to types and dosages of therapeutic or nutritional agents has become an important issue in clinical medicine today not only from the aspect of efficiency of medical practice for the health care industry but for improved outcomes and benefits for the patients.

### SUMMARY OF THE INVENTION

In a first embodiment there is provided a method for determining a profile data set for a subject with infectious disease or inflammatory conditions related to infectious disease based on a sample from the subject, the sample providing a source of RNAs, the method comprising using amplification for measuring the amount of RNA corresponding to at least 2 constituents from Table 1 and arriving at a measure of each constituent, wherein the profile data set comprises the measure of each constituent and wherein amplification is performed under measurement conditions that are substantially repeatable.

In addition, the subject may have presumptive signs of a systemic infection including at least one of elevated white blood cell count, elevated temperature, elevated heart rate, and elevated or reduced blood pressure, relative to medical standards or the inflammatory conditions related to infectious disease may be inflammatory conditions arising from at least one of blunt or penetrating trauma, surgery, endocarditis, urinary tract infection, pneumonia, or dental or gynecological examinations or treatments.

In other embodiments, the measurement conditions that are substantially repeatable may be within a degree of repeatability of better than five percent, or better than three percent and the efficiencies of amplification for all constituents may be substantially similar wherein the efficiency of amplification for all constituents is within two percent, or alternatively, is less than one percent. In such embodiments, the sample may be selected from the group consisting of blood, a blood fraction, body fluid, a population of cells and tissue from the subject.

In another embodiment there is provided a method of characterizing infectious disease or inflammatory conditions related to infectious disease in a subject, based on a sample from the subject, the sample providing a source of RNAs, the method comprising assessing a profile data set of a plurality of members, each member being a quantitative measure of the amount of a distinct RNA constituent in a panel of constituents selected so that measurement of the constituents enables characterization of the presumptive signs of a systemic infection, wherein such measure for each constituent is obtained under measurement conditions that are substantially repeatable.

In addition, the subject may have presumptive signs of a systemic infection including at least one of elevated white blood cell count, elevated temperature, elevated heart rate, and elevated or reduced blood pressure, relative to medical standards, or alternatively, the subject may have presumptive signs of a systemic infection that are related to inflammatory conditions arising from at least one of blunt or penetrating trauma, surgery, endocarditis, urinary tract infection, pneumonia, or dental or gynecological examinations or treatments. In such embodiments, assessing may further comprises comparing the profile data set to a baseline profile data set for the panel, wherein the baseline profile data set is related to the infectious disease or inflammatory conditions related to infectious disease to be characterized.

In other embodiments, the efficiencies of amplification for all constituents are substantially similar and the infectious disease or inflammatory conditions related to infectious disease are from a microbial infection, more particularly a bacterial infection, or a eukaryotic parasitic infection, or a viral infection, or a fungal infection or are related to systemic inflammatory response syndrome (SIRS). More particularly, the infectious disease or inflammatory conditions that are related to infectious disease may be from bacteremia, viremia, or fungemia, or from septicemia due to any class of microbe. In addition, the infectious disease or inflammatory conditions related to infectious disease may be with respect to a localized tissue of the subject and the sample may be derived from a tissue or fluid of a type distinct from that of the localized tissue.

Other embodiments include storing the profile data set in a digital storage medium, wherein storing the profile data set may include storing it as a record in a database.

Yet another embodiment provides a method for evaluating infectious disease or inflammatory conditions related to infectious disease in a subject based on a first sample from the subject, the sample providing a source of RNAs, the method comprising deriving from the first sample a first profile data set, the profile data set including a plurality of members, each member being a quantitative measure of the amount of a distinct RNA constituent in a panel of constituents selected so that measurement of the constituents enables evaluation of the infectious disease or inflammatory conditions related to infectious disease wherein such measure for each constituent is obtained under measurement conditions that are substantially repeatable. The method also includes producing a calibrated profile data set for the panel, wherein each member of the calibrated profile data set is a function of a corresponding member of the first profile data set and a corresponding member of a baseline profile data set for the panel, and wherein the baseline profile data set is related to the infectious disease or inflammatory conditions related to infectious disease to be evaluated, with the calibrated profile data set being a comparison between the first profile data set and the baseline profile data set, thereby providing evaluation of the infectious disease or inflammatory conditions related to infectious disease of the subject.

In related embodiments, the subject has presumptive signs of a systemic infection including at least one of: elevated white blood cell count, elevated temperature, elevated heart rate, and elevated or reduced blood pressure, relative to medical standards, or alternatively, the infectious disease or inflammatory conditions may be related to inflammatory conditions arising from at least one of blunt or penetrating trauma, surgery, endocarditis, urinary tract infection, pneumonia, or dental or gynecological examinations or treatments.

In addition, the baseline profile data set may be derived from one or more other samples from the same subject taken under circumstances different from those of the first sample, and the circumstances may be selected from the group consisting of (i) the time at which the first sample is taken, (ii) the site from which the first sample is taken, (iii) the biological condition of the subject when the first sample is taken.

Also, the one or more other samples may be taken over an interval of time that is at least one month between the first sample and the one or more other samples, or taken over an interval of time that is at least twelve months between the first sample and the one or more samples, or they may be taken pre-therapy intervention or post-therapy intervention. In such embodiments, the first sample may be derived from blood and the baseline profile data set may be derived from tissue or body fluid of the subject other than blood. Alternatively, the first sample is derived from tissue or body fluid of the subject and the baseline profile data set is derived from blood.

In other embodiments, the baseline profile data set may be derived from one or more other samples from the same subject, taken when the subject is in a biological condition different from that in which the subject was at the time the first sample was taken, with respect to at least one of age, nutritional history, medical condition, clinical indicator, medication, physical activity, body mass, and environmental exposure, and the baseline profile data set may be derived from one or more other samples from one or more different subjects.

In addition, the one or more different subjects may have in common with the subject at least one of age group, gender, ethnicity, geographic location, nutritional history, medical condition, clinical indicator, medication, physical activity, body mass, and environmental exposure. In other embodiments, a clinical indicator may be used to assess infectious disease or inflammatory conditions related to infectious disease of the one or more different subjects, and may also include interpreting the calibrated profile data set in the context of at least one other clinical indicator, wherein the at least one other clinical indicator is selected from the group consisting of blood chemistry, urinalysis, X-ray or other radiological or metabolic imaging technique, other chemical assays, and physical findings.

In such embodiments, the infectious disease or inflammatory conditions related to infectious disease may be from a microbial infection, a bacterial infection, a eukaryotic parasitic infection, a viral infection, a fungal infection, or alternatively, the infectious disease or inflammatory conditions related to infectious disease may be from systemic inflammatory response syndrome (SIRS), from bacteremia, viremia, fungemia, or septicemia due to any class of microbe.

In yet other embodiments, the function is a mathematical function and is other than a simple difference, including a second function of the ratio of the corresponding member of first profile data set to the corresponding member of the baseline profile data set, or a logarithmic function. In related embodiments, each member of the calibrated profile data set has biological significance if it has a value differing by more than an amount D, where D=F(1.1)−F(0.9), and F is the second function. In such embodiments, the first sample is obtained and the first profile data set quantified at a first location, and the calibrated profile data set is produced using a network to access a database stored on a digital storage medium in a second location, wherein the database may be updated to reflect the first profile data set quantified from the sample. Additionally, using a network may include accessing a global computer network.

In related embodiments, the quantitative measure is determined by amplification, and the measurement conditions are such that efficiencies of amplification for all constituents differ by less than approximately 2 percent, or alternatively by less than approximately 1 percent.

Still another embodiment is a method of providing an index that is indicative of infectious disease or inflammatory conditions related to infectious disease of a subject based on a first sample from the subject, the first sample providing a source of RNAs, the method comprising deriving from the first sample a profile data set, the profile data set including a plurality of members, each member being a quantitative measure of the amount of a distinct RNA constituent in a panel of constituents selected so that measurement of the constituents is indicative of the presumptive signs of a systemic infection, the panel including at least two of the constituents of the Gene Expression Panel of Table 1. In deriving the profile data set, such measure for each constituent is achieved under measurement conditions that are substantially repeatable, at least one measure from the profile data set is applied to an index function that provides a mapping from at least one measure of the profile data set into one measure of the presumptive signs of a systemic infection, so as to produce an index pertinent to the infectious disease or inflammatory conditions related to infectious disease of the subject.

In addition, the subject may have presumptive signs of a systemic infection including at least one of: elevated white blood cell count, elevated temperature, elevated heart rate, and elevated or reduced blood pressure, relative to medical standards, or alternatively, the infectious disease or inflammatory conditions may be related to inflammatory conditions arising from at least one of blunt or penetrating trauma, surgery, endocarditis, urinary tract infection, pneumonia, or dental or gynecological examinations or treatments.

In related embodiments, the index function is constructed as a linear sum of terms having the form: I=ΣCᵢMᵢ^{P(i)}, wherein I is the index, Mᵢ is the value of the member i of the profile data set, Cᵢ is a constant, and P(i) is a power to which Mᵢ is raised, the sum being formed for all integral values of i up to the number of members in the data set. In addition, the values Cᵢ and P(i) are determined using statistical techniques, such as latent class modeling, to correlate data, including clinical, experimentally derived, and any other data pertinent to the presumptive signs of a systemic infection. In alternative embodiments, there is provided a normative value of the index function, determined with respect to a relevant set of subjects, so that the index may be interpreted in relation to the normative value, wherein the normative value may include constructing the index function so that the normative value is approximately 1, alternatively so that the normative value is approximately 0 and deviations in the index function from 0 are expressed in standard deviation units. In still other embodiments, the relevant set of subjects has in common a property that is at least one of age group, gender, ethnicity, geographic location, nutritional history, medical condition, clinical indicator, medication, physical activity, body mass, and environmental exposure, or alternatively has in common a property that is at least one of age group, gender, ethnicity, geographic location, nutritional history, medical condition, clinical indicator, medication, physical activity, body mass, and environmental exposure.

In other embodiments, a clinical indicator may be used to assess the infectious disease or inflammatory conditions related to infectious disease of the relevant set of subjects by interpreting the calibrated profile data set in the context of at least one other clinical indicator, wherein the at least one other clinical indicator is selected from the group consisting of blood chemistry, urinalysis, X-ray or other radiological or metabolic imaging technique, other chemical assays, and physical findings. In addition, the quantitative measure may be determined by amplification, the measurement conditions being such that efficiencies of amplification for all constituents differ by less than approximately 2 percent, or they differ by less than approximately 1 percent, and the measurement conditions that are substantially repeatable are within a degree of repeatability of better than five percent, or within a degree of repeatability of better than three percent.

In such embodiments, the infectious disease or inflammatory conditions related to infectious disease being evaluated are with respect to a localized tissue of the subject and the first sample is derived from tissue or fluid of a type distinct from that of the localized tissue, wherein the infectious disease or inflammatory conditions related to infectious disease are from a microbial infection, more particularly a bacterial infection, still more particularly a eukaryotic parasitic infection, a viral infection, a fungal infection or from a systemic inflammatory response syndrome (SIRS).

A method of providing an index according to claim 61, further comprising:

deriving from at least one other sample at least one other profile data set, the at least one other profile data set including a plurality of members, each being a quantitative measure of the amount of a distinct RNA constituent in a panel of constituents selected so that measurement of the constituents is indicative of the presumptive signs of a systemic infection,

wherein the at least one other sample is from the same subject, taken under circumstances different from those of the first sample with respect to at least one of time, nutritional history, medical condition, clinical indicator, medication, physical activity, body mass, and environmental exposure; and

applying at least one measure from the at least one other profile data set to an index function that provides a mapping from the at least one measure of the at least one other profile data set into one measure of the infectious disease or inflammatory conditions related to infectious disease under different circumstances, so as to produce at least one other index pertinent to the infectious disease or inflammatory conditions related to infectious disease of the subject under circumstances different from those of the first sample.

Related embodiments include providing an index wherein the index function has 2, 3, 4, or 5 components including disease status, disease severity, or disease course. In addition, the index function may be constructed as a linear sum of terms having the form: I=ΣCᵢMᵢ^{P(i)}, wherein I is the index, Mᵢ is the value of the member i of the profile data set, Cᵢ is a constant, and P(i) is a power to which Mᵢ is raised, the sum being formed for all integral values of i up to the number of members in the data set, wherein the values Cᵢ and P(i) are determined using statistical techniques, such as latent class modeling, to correlate data, including clinical, experimentally derived, and any other data pertinent to the presumptive signs of a systemic infection.

Alternatively, a normative value of the index function is provided, determined with respect to a relevant set of subjects, so that the at least one other index may be interpreted in relation to the normative value, wherein providing the normative value includes constructing the index function so that the normative value is approximately 1, or so that the normative value is approximately 0 and deviations in the index function from 0 are expressed in standard deviation units. Such embodiments may also include using a clinical indicator to assess infectious disease or inflammatory conditions related to infectious disease of the relevant set of subjects by interpreting the calibrated profile data set in the context of at least one other clinical indicator selected from the group consisting of blood chemistry, urinalysis, X-ray or other radiological or metabolic imaging technique, other chemical assays, and physical findings.

As in other embodiments, the quantitative measure is determined by amplification, and the measurement conditions are such that efficiencies of amplification for all constituents differ by less than approximately 2 percent, or differ by less than approximately 1 percent, and the measurement conditions that are substantially repeatable are within a degree of repeatability of better than five percent or within a degree of repeatability of better than three percent.

In addition, the infectious disease or inflammatory conditions related to infectious disease are with respect to a localized tissue of the subject and the first sample is derived from tissue or fluid of a type distinct from that of the localized tissue.

Still other embodiments include a method for providing an index wherein the infectious disease or inflammatory conditions related to infectious disease are from a microbial infection, a bacterial infection, a viral infection, a fungal infection, a eukaryotic parasite infection, or from systemic inflammatory response syndrome (SIRS) and the panel of constituents includes at least two constituents of Table 1.

Another embodiment provides a method for evaluating infectious disease or inflammatory conditions related to infectious disease of a subject based on a first sample from the subject, the first sample providing a source of RNAs, the method comprising deriving from the first sample a first profile data set, the first profile data set including a plurality of members, each member being a quantitative measure of the amount of a distinct RNA constituent in a panel of constituents selected so that measurement of the constituents enables evaluation of the infectious disease or inflammatory conditions related to infectious disease wherein such measure for each constituent is obtained under measurement conditions that are substantially repeatable. The method also includes producing a calibrated profile data set for the panel, wherein each member of the calibrated profile data set is a function of a corresponding member of the first profile data set and a corresponding member of a baseline profile data set for the panel, wherein each member of the baseline profile data set is a normative measure determined with respect to a relevant set of subjects of the amount of one of the constituents in the panel and the baseline profile data set is related to the infectious disease or inflammatory conditions related to infectious disease to be evaluated, and the calibrated profile data set is a comparison between the first profile data set and the baseline profile data set, thereby providing evaluation of the infectious disease or inflammatory conditions related to infectious disease of the subject.

In such an embodiment, the subject may have presumptive signs of a systemic infection including at least one of: elevated white blood cell count, elevated temperature, elevated heart rate, and elevated or reduced blood pressure, relative to medical standards, or the infectious disease or inflammatory conditions may be related to inflammatory conditions arising from at least one of: blunt or penetrating trauma, surgery, endocarditis, urinary tract infection, pneumonia, or dental or gynecological examinations or treatments.

Additionally, the relevant set of subjects is a set of healthy subjects having in common a property that is at least one of age group, gender, ethnicity, geographic location, nutritional history, medical condition, clinical indicator, medication, physical activity, body mass, and environmental exposure. As with other embodiments, the quantitative measure is determined by amplification, and the measurement conditions are such that efficiencies of amplification for all constituents differ by less than approximately 2 percent, or they differ by less than approximately 1 percent, and the measurement conditions are substantially repeatable within a degree of repeatability of better than five percent or within a degree of repeatability of better than three percent.

In such embodiments, the infectious disease or inflammatory conditions related to infectious disease being evaluated is with respect to a localized tissue of the subject and the first sample is derived from tissue or fluid of a type distinct from that of the localized tissue and the profile data set may be stored in a digital storage medium, including storing it as a record in a database. In addition, the baseline profile data set is derived from one or more other samples from the same subject taken under circumstances different from those of the first sample, wherein the one or more other samples are taken pre-therapy intervention or alternatively taken post-therapy intervention, or the one or more other samples are taken over an interval of time that is at least one month between an initial sample and the sample, or at least twelve months between an initial sample and the sample. Also, the first sample is derived from blood and the baseline profile data set is derived from tissue or body fluid of the subject other than blood, or alternatively, the first sample is derived from tissue or body fluid of the subject and the baseline profile data set is derived from blood.

Yet another embodiment provides a method for evaluating infectious disease or inflammatory conditions related to infectious disease of a subject based on a first sample from the subject and a second sample from a defined population of indicator cells, the samples providing a source of RNAs, the method comprising applying the first sample or a portion thereof to the defined population of indicator cells. The method also includes deriving from the second sample a first profile data set, the first profile data set including a plurality of members, each member being a quantitative measure of the amount of a distinct RNA or protein constituent in a panel of constituents selected so that measurement of the constituents enables measurement of the presumptive signs of a systemic infection, wherein such measure for each constituent is obtained under measurement conditions that are substantially repeatable, and also includes producing a calibrated profile data set for the panel, wherein each member of the calibrated profile data set is a function of a corresponding member of the first profile data set and a corresponding member of a baseline profile data set for the panel, wherein each member of the baseline data set is a normative measure determined with respect to a relevant set of subjects of the amount of one of the constituents in the panel and wherein the baseline profile data set is related to the infectious disease or inflammatory conditions related to infectious disease to be evaluated, the calibrated profile data set being a comparison between the first profile data set and the baseline profile data set, thereby providing evaluation of the infectious disease or inflammatory conditions related to infectious disease of the subject.

In related embodiments, the subject may have presumptive signs of a systemic infection including at least one of: elevated white blood cell count, elevated temperature, elevated heart rate, and elevated or reduced blood pressure, relative to medical standards, or alternatively, the infectious disease or inflammatory conditions may be related to inflammatory conditions arising from at least one of: blunt or penetrating trauma, surgery, endocarditis, urinary tract infection, pneumonia, or dental or gynecological examinations or treatments, and the relevant set of subjects is a set of healthy subjects.

In addition, the relevant set of subjects has in common a property that is at least one of age group, gender, ethnicity, geographic location, nutritional history, medical condition, clinical indicator, medication, physical activity, body mass, and environmental exposure. Additionally, a clinical indicator may be used to assess infectious disease or inflammatory conditions-related to infectious disease of the relevant set of subjects by interpreting the calibrated profile data set in the context of at least one other clinical indicator, wherein the at least one other clinical indicator is selected from the group consisting of blood chemistry, urinalysis, X-ray or other radiological or metabolic imaging technique, other chemical assays, and physical findings.

As with other embodiments, the quantitative measure is determined by amplification, and the measurement conditions are such that efficiencies of amplification for all constituents differ by less than approximately 2 percent, or they differ by less than approximately 1 percent, and the measurement conditions are substantially repeatable within a degree of repeatability of better than five percent, or within a degree of repeatability of better than three percent. Also, the infectious disease being evaluated is with respect to a localized tissue of the subject and the first sample is derived from tissue or fluid of a type distinct from that of the localized tissue, and the infectious disease or inflammatory conditions related to infectious disease is a microbial infection.

In related embodiments, the baseline profile data set is derived from one or more other samples from the same subject taken under circumstances different from those of the first sample, wherein the one or more other samples are taken pre-therapy intervention, or are taken post-therapy intervention, or are taken over an interval of time that is at least one month between an initial sample and the sample, or are taken over an interval of time that is at least twelve months between an initial sample and the sample. In such embodiments, the first sample is derived from blood and the baseline profile data set is derived from tissue or body fluid of the subject other than blood, or the first sample is derived from tissue or body fluid of the subject and the baseline profile data set is derived from blood.

In another embodiment of the invention, a method for evaluating infectious disease or inflammatory conditions related to infectious disease of a target population of cells affected by a first agent, based on a sample from the target population of cells to which the first agent has been administered, the sample providing a source of RNAs, is presented. The method comprises deriving from the sample a first profile data set, the first profile data set including a plurality of members, each member being a quantitative measure of the amount of a distinct RNA constituent in a panel of constituents selected so that measurement of the constituents enables evaluation of the infectious disease or inflammatory conditions related to infectious disease affected by the first agent, wherein such measure for each constituent is obtained under measurement conditions that are substantially repeatable; and producing a calibrated profile data set for the panel, wherein each member of the calibrated profile data set is a function of a corresponding member of the first profile data set and a corresponding member of a baseline profile data set for the panel, wherein each member of the baseline data set is a normative measure determined with respect to a relevant set of target populations of cells of the amount of one of the constituents in the panel, and wherein the baseline profile data set is related to the infectious disease or inflammatory conditions related to infectious disease to be evaluated, the calibrated profile data set being a comparison between the first profile data set and the baseline profile data set, thereby providing an evaluation of the infectious disease or inflammatory conditions related to infectious disease of the target population of cells affected by the first agent. The target population of cells may have presumptive signs of a systemic infection including at least one of: elevated white blood cell count, elevated temperature, elevated heart rate, and elevated or reduced blood pressure, relative to medical standards. The infectious disease or inflammatory conditions related to infectious disease may be related to inflammatory conditions arising from at least one of: blunt or penetrating trauma, surgery, endocarditis, urinary tract infection, pneumonia, or dental or gynecological examinations or treatments. The relevant set of target populations of cells may be a set of healthy target populations of cells. Alternatively, the relevant set of target populations of cells may have in common a property that is at least one of age group, gender, ethnicity, geographic location, nutritional history, medical condition, clinical indicator, medication, physical activity, body mass, and environmental exposure. In such a case, a clinical indicator may be used to assess infectious disease or inflammatory conditions related to infectious disease of the relevant set of target populations of cells, and the method further comprises interpreting the calibrated profile data set in the context of at least one other clinical indicator; the at least one other clinical indicator may be selected from the group consisting of blood chemistry, urinalysis, X-ray or other radiological or metabolic imaging technique, other chemical assays, and physical findings. The quantitative measure may be determined by amplification, and the measurement conditions are such that efficiencies of amplification for all constituents differ by less than approximately 2 percent, or alternatively, less than approximately 1 percent. The measurement conditions that are substantially repeatable may be within a degree of repeatability of better than five percent, or alternatively better than three percent. Also, the infectious disease or inflammatory conditions related to infectious disease being evaluated may be with respect to a localized tissue of the subject and the first sample is derived from tissue or fluid of a type distinct from that of the localized tissue. The infectious disease or inflammatory conditions related to infectious disease may be a microbial infection, a bacterial infection, a eukaryotic parasitic infection, a viral infection, a fungal infection, systemic inflammatory response syndrome (SIRS), bacteremia, viremia, fungemia, or septicemia due to any class of microbe. A related embodiment of the method may further comprise storing the profile data set in a digital storage medium. Storing the profile data set may include storing it as a record in a database. The embodiment may include the limitations that the first sample is derived from blood and the baseline profile data set is derived from tissue or body fluid of the subject other than blood. Alternatively, the first sample may be derived from tissue or body fluid of the subject and the baseline profile data set is derived from blood. As well, the baseline profile data set may be derived from one or more other samples from the same subject taken under circumstances different from those of the first sample. Such one or more other samples may be taken pre-therapy intervention, post-therapy intervention, or over an interval of time that is at least one month between an initial sample and the sample.

Other embodiments of the invention are directed toward a method for evaluating infectious disease or inflammatory conditions related to infectious disease of a target population of cells affected by a first agent in relation to the infectious disease or inflammatory conditions related to infectious disease of the target population of cells affected by a second agent, based on a first sample from the target population cells to which the first agent has been administered and a second sample from the target population of cells to which the second agent has been administered, the samples providing a source of RNAs. Such a method includes the steps of deriving from the first sample a first profile data set and from the second sample a second profile data set, the first and second profile data sets each including a plurality of members, each member being a quantitative measure of the amount of a distinct RNA constituent in a panel of constituents selected so that measurement of the constituents enables evaluation of the infectious disease or inflammatory conditions related to infectious disease affected by the first agent in relation to the second agent, wherein such measure for each constituent is obtained under measurement conditions that are substantially repeatable; and producing a first calibrated profile data set and a second calibrated profile data set for the panel, wherein (i) each member of the first calibrated profile data set is a function of a corresponding member of the first profile data set and a corresponding member of a baseline profile data set for the panel, and (ii) each member of the second calibrated profile data set is a function of a corresponding member of the second profile data set and a corresponding member of the baseline profile data set, wherein each member of the baseline data set is a normative measure, determined with respect to a relevant set of subjects, of the amount of one of the constituents in the panel, and wherein the baseline profile data set is related to the infectious disease or inflammatory conditions related to infectious disease to be evaluated, the first and second calibrated profile data sets being a comparison between the first profile data set and the baseline profile set and a comparison between the second profile data set and the baseline profile data set, thereby providing an evaluation of the infectious disease or inflammatory conditions related to infectious disease of the target population of cells affected by the first agent in relation to the infectious disease or inflammatory conditions related to infectious disease of the target population of cells affected by the second agent. The target population of cells may have presumptive signs of a systemic infection including at least one of: elevated white blood cell count, elevated temperature, elevated heart rate, and elevated or reduced blood pressure, relative to medical standards. As well, the target population of cells may have presumptive signs of a systemic infection that are related to inflammatory conditions arising from at least one of: blunt or penetrating trauma, surgery, endocarditis, urinary tract infection, pneumonia, or dental or gynecological examinations or treatments. The first agent may be a first drug and the second agent may be a second drug. Alternatively, the first agent is a drug and the second agent is a complex mixture or a nutriceutical. The quantitative measure may be determined by amplification, and the measurement conditions are such that efficiencies of amplification for all constituents differ by less than approximately 2 percent, or alternatively by less than approximately 1 percent. The measurement conditions that are substantially repeatable may be within a degree of repeatability of better than five percent, or alternatively better than three percent. The infectious disease or inflammatory conditions related to infectious disease being evaluated may be with respect to a localized tissue of the subject and the first sample is derived from tissue or fluid of a type distinct from that of the localized tissue. The infectious disease or inflammatory conditions related to infectious disease may be a microbial infection, bacterial infection, a eukaryotic parasitic infection, a viral infection, a fungal infection, systemic inflammatory response syndrome (SIRS), bacteremia, viremia, fungemia, or septicemia due to any class of microbe. This method may further include the step of storing the first and second profile data sets in a digital storage medium. The first and second profile data sets may include storing each data set as a record in a database. The baseline profile data set may be derived from one or more other samples from the same subject taken under circumstances different from those of the first sample, or alternatively different from those of the second sample. The first sample may be derived from blood and the baseline profile data set may be derived from tissue or body fluid of the subject other than blood. The first sample may be derived from tissue or body fluid of the subject and the baseline profile data set may be derived from blood.

In yet another embodiment of the invention, a method of providing an index that is indicative of an inflammatory condition of a subject with presumptive signs of a systemic infection, based on a first sample from the subject, the first sample providing a source of RNAs, is presented. The method comprises deriving from the first sample a profile data set, the profile data set including a plurality of members, each member being a quantitative measure of the amount of a distinct RNA constituent in a panel of constituents selected so that measurement of the constituents is indicative of the inflammatory condition, the panel including at least two of the constituents of the Gene Expression Panel of Table 1; and in deriving the profile data set, achieving such measure for each constituent under measurement conditions that are substantially repeatable; applying at least one measure from the profile data set to an index function that provides a mapping from at least one measure of the profile data set into at least one measure of the inflammatory condition, so as to produce an index pertinent to the inflammatory condition of the sample; wherein the index function uses data from a baseline profile data set for the panel, each member of the baseline data set being a normative measure, determined with respect to a relevant set of subjects, of the amount of one of the constituents in the panel, wherein the baseline data set is related to the inflammatory condition to be evaluated. The subject may have presumptive signs of a systemic infection including at least one of: elevated white blood cell count, elevated temperature, elevated heart rate, and elevated or reduced blood pressure, relative to medical standards. Alternatively, the presumptive signs of a systemic infection are related to inflammatory conditions arising from at least one of: blunt or penetrating trauma, surgery, endocarditis, urinary tract infection, pneumonia, or dental or gynecological examinations or treatments. The at least one measure of the profile data set that is applied to the index function may be 2, 3, 4, or 5.

Still other embodiments provide a method of using an index to direct therapy intervention in a subject with infectious disease or inflammatory conditions related to infectious disease, the method comprising providing an index according to any of the above-discussed embodiments, comparing the index to a normative value of the index, determined with respect to a relevant set of subjects to obtain a difference, and using the difference between the index and the normative value for the index to direct therapy intervention, wherein therapy intervention is microbe-specific therapy, or is bacteria-specific therapy, or is fungus-specific therapy, or is virus-specific therapy, or is eukaryotic parasite-specific therapy.

Another embodiment provides a method for differentiating a type of pathogen within a class of pathogens of interest in a subject with infectious disease or inflammatory conditions related to infectious disease, based on at least one sample from the subject, the sample providing a source of RNA, the method comprising: determining at least one profile data set for the subject, comparing the profile data set to at least one baseline profile data set, determined with respect to at least one relevant set of samples within the class of pathogens of interest to obtain a difference, and using the difference to differentiate the type of pathogen in the at least one profile data set for the subject from the class of pathogen in the at least one baseline profile data set, wherein the class of pathogens is microbial. Alternatively, the class of pathogens is bacterial and the difference is used to differentiate a Gram(+) bacterial pathogen from a Gram(−) bacterial pathogen. Alternatively, the class of pathogens is fungal and the difference is used to differentiate an acute Candida pathogen from a chronic Candida pathogen. More particularly, the class of pathogens is viral and the difference is used to differentiate a DNA viral pathogen from an RNA viral pathogen, or the class of pathogens is viral and the difference is used to differentiate a rhinovirus pathogen from an influenza pathogen. Still more particularly, the class of pathogens is eukaryotic parasites and the difference is used to differentiate a plasmodium parasite pathogen from a trypanosomal pathogen.

Yet another embodiment provides a method of using an index for differentiating a type of pathogen within a class of pathogens of interest in a subject with infectious disease or inflammatory conditions related to infectious disease, based on at least one sample from the subject, the method comprising providing at least one index according to any of the above disclosed embodiments for the subject, comparing the at least one index to at least one normative value of the index, determined with respect to at least one relevant set of subjects to obtain at least one difference, and using the at least one difference between the at least one index and the at least one normative value for the index to differentiate the type of pathogen from the class of pathogen.

### BRIEF DESCRIPTION OF THE DRAWINGS

The foregoing features of the invention will be more readily understood by reference to the following detailed description, taken with reference to the accompanying drawings, in which:

FIG. 1A shows the results of assaying 24 genes from the Source Inflammation Gene Panel (shown in Table 1) on eight separate days during the course of optic neuritis in a single male subject.

FIG. 1B illustrates use of an inflammation index in relation to the data of FIG. 1A, in accordance with an embodiment of the present invention.

FIG. 2 is a graphical illustration of the same inflammation index calculated at 9 different, significant clinical milestones.

FIG. 3 shows the effects of single dose treatment with 800 mg of ibuprofen in a single donor as characterized by the index.

FIG. 4 shows the calculated acute inflammation index displayed graphically for five different conditions.

FIG. 5 shows a Viral Response Index for monitoring the progress of an upper respiratory infection (URI).

FIGS. 6 and 7 compare two different populations using Gene Expression Profiles (with respect to the 48 loci of the Inflammation Gene Expression Panel of Table 1).

FIG. 8 compares a normal population with a rheumatoid arthritis population derived from a longitudinal study.

FIG. 9 compares two normal populations, one longitudinal and the other cross sectional.

FIG. 10 shows the shows gene expression values for various individuals of a normal population.

FIG. 11 shows the expression levels for each of four genes (of the Inflammation Gene Expression Panel of Table 1), of a single subject, assayed monthly over a period of eight months.

FIGS. 12 and 13 similarly show in each case the expression levels for each of 48 genes (of the Inflammation Gene Expression Panel of Table 1), of distinct single subjects (selected in each case on the basis of feeling well and not taking drugs), assayed, in the case of FIG. 12 weekly over a period of four weeks, and in the case of FIG. 13 monthly over a period of six months.

FIG. 14 shows the effect over time, on inflammatory gene expression in a single human subject, of the administration of an anti-inflammatory steroid, as assayed using the Inflammation Gene Expression Panel of Table 1.

FIG. 15, in a manner analogous to FIG. 14, shows the effect over time, via whole blood samples obtained from a human subject, administered a single dose of prednisone, on expression of 5 genes (of the Inflammation Gene Expression Panel of Table 1).

FIG. 16 also shows the effect over time, on inflammatory gene expression in a single human subject suffering from rheumatoid arthritis, of the administration of a TNF-inhibiting compound, but here the expression is shown in comparison to the cognate locus average previously determined (in connection with FIGS. 6 and 7) for the normal (i.e., undiagnosed, healthy) population.

FIG. 17A further illustrates the consistency of inflammatory gene expression in a population.

FIG. 17B shows the normal distribution of index values obtained from an undiagnosed population.

FIG. 17C illustrates the use of the same index as FIG. 17B, where the inflammation median for a normal population has been set to zero and both normal and diseased subjects are plotted in standard deviation units relative to that median.

FIG. 18 plots, in a fashion similar to that of FIG. 17A, Gene Expression Profiles, for the same 7 loci as in FIG. 17A, two different (responder v. non-responder) 6-subject populations of rheumatoid arthritis patients.

FIG. 19 thus illustrates use of the inflammation index for assessment of a single subject suffering from rheumatoid arthritis, who has not responded well to traditional therapy with methotrexate.

FIG. 20 similarly illustrates use of the inflammation index for assessment of three subjects suffering from rheumatoid arthritis, who have not responded well to traditional therapy with methotrexate.

FIGS. 21-23 show the inflammation index for an international group of subjects, suffering from rheumatoid arthritis, undergoing three separate treatment regimens.

FIG. 24 illustrates use of the inflammation index for assessment of a single subject suffering from inflammatory bowel disease.

FIG. 25 shows Gene Expression Profiles with respect to 24 loci (of the Inflammation Gene Expression Panel of Table 1) for whole blood treated with Ibuprofen in vitro in relation to other non-steroidal anti-inflammatory drugs (NSAIDs).

FIGS. 26A-26D illustrate how the effects of two competing anti-inflammatory compounds can be compared objectively, quantitatively, precisely, and reproducibly.

FIGS. 27 through 41 illustrate the use of gene expression panels in early identification and monitoring of infectious disease.

FIG. 27 uses a novel bacterial Gene Expression Panel of 24 genes, developed to discriminate various bacterial conditions in a host biological system.

FIG. 28 shows differential expression for a single locus, IFNG, to LTA derived from three distinct sources: S. pyogenes, B. subtilis, and S. aureus.

FIGS. 29 and 30 show the response after two hours of the Inflammation 48A and 48B loci respectively (discussed above in connection with FIGS. 6 and 7 respectively) in whole blood to administration of a Gram-positive and a Gram-negative organism.

FIGS. 31 and 32 correspond to FIGS. 29 and 30 respectively and are similar to them, with the exception that the monitoring here occurs 6 hours after administration.

FIG. 33 compares the gene expression response induced by E. coli and by an organism-free E. coli filtrate.

FIG. 34 is similar to FIG. 33, but here the compared responses are to stimuli from E. coli filtrate alone and from E. coli filtrate to which has been added polymyxin B.

FIG. 35 illustrates the gene expression responses induced by S. aureus at 2, 6, and 24 hours after administration.

FIGS. 36 through 41 compare the gene expression induced by E. coli and S. aureus under various concentrations and times.

FIG. 42 illustrates application of a statistical T-test to identify potential members of a signature gene expression panel that is capable of distinguishing between normal subjects and subjects suffering from unstable rheumatoid arthritis.

FIG. 43 illustrates, for a panel of 17 genes, the expression levels for 8 patients presumed to have bacteremia.

FIG. 44 illustrates application of a statistical T-test to identify potential members of a signature gene expression panel that is capable of distinguishing between normal subjects and subjects suffering from bacteremia FIG. 45 illustrates application of an algorithm (shown in the figure), providing an index pertinent to rheumatoid arthritis (RA) as applied respectively to normal subjects, RA patients, and bacteremia patients.

FIG. 46 illustrates application of an algorithm (shown in the figure), providing an index pertinent to bacteremia as applied respectively to normal subjects, rheumatoid arthritis patients, and bacteremia patients.

### DETAILED DESCRIPTION OF SPECIFIC EMBODIMENTS

### Definitions

The following terms shall have the meanings indicated unless the context otherwise requires:

“Algorithm” is a set of rules for describing a biological condition. The rule set may be defined exclusively algebraically but may also include alternative or multiple decision points requiring domain-specific knowledge, expert interpretation or other clinical indicators.

An “agent” is a “composition” or a “stimulus”, as those terms are defined herein, or a combination of a composition and a stimulus.

“Amplification” in the context of a quantitative RT-PCR assay is a function of the number of DNA replications that are tracked to provide a quantitative determination of its concentration. “Amplification” here refers to a degree of sensitivity and specificity of a quantitative assay technique. Accordingly, amplification provides a measurement of concentrations of constituents that is evaluated under conditions wherein the efficiency of amplification and therefore the degree of sensitivity and reproducibility for measuring all constituents is substantially similar.

A “baseline profile data set” is a set of values associated with constituents of a Gene Expression Panel resulting from evaluation of a biological sample (or population or set of samples) under a desired biological condition that is used for mathematically normative purposes. The desired biological condition may be, for example, the condition of a subject (or population or set of subjects) before exposure to an agent or in the presence of an untreated disease or in the absence of a disease. Alternatively, or in addition, the desired biological condition may be health of a subject or a population or set of subjects. Alternatively, or in addition, the desired biological condition may be that associated with a population or set of subjects selected on the basis of at least one of age group, gender, ethnicity, geographic location, nutritional history, medical condition, clinical indicator, medication, physical activity, body mass, and environmental exposure.

A “set” or “population” of samples or subjects refers to a defined or selected group of samples or subjects wherein there is an underlying commonality or relationship between the members included in the set or population of samples or subjects.

A “population of cells” refers to any group of cells wherein there is an underlying commonality or relationship between the members in the population of cells, including a group of cells taken from an organism or from a culture of cells or from a biopsy, for example,

A “biological condition” of a subject is the condition of the subject in a pertinent realm that is under observation, and such realm may include any aspect of the subject capable of being monitored for change in condition, such as health, disease including cancer, trauma; aging; infection; tissue degeneration; developmental steps; physical fitness; obesity, and mood. As can be seen, a condition in this context may be chronic or acute or simply transient. Moreover, a targeted biological condition may be manifest throughout the organism or population of cells or may be restricted to a specific organ (such as skin, heart, eye or blood), but in either case, the condition may be monitored directly by a sample of the affected population of cells or indirectly by a sample derived elsewhere from the subject. The term “biological condition” includes a “physiological condition”.

“Body fluid” of a subject includes blood, urine, spinal fluid, lymph, mucosal secretions, prostatic fluid, semen, haemolymph or any other body fluid known in the art for a subject.

“Calibrated profile data set” is a function of a member of a first profile data set and a corresponding member of a baseline profile data set for a given constituent in a panel.

A “clinical indicator” is any physiological datum used alone or in conjunction with other data in evaluating the physiological condition of a collection of cells or of an organism. This term includes pre-clinical indicators.

A “composition” includes a chemical compound, a nutriceutical, a pharmaceutical, a homeopathic formulation, an allopathic formulation, a naturopathic formulation, a combination of compounds, a toxin, a food, a food supplement, a mineral, and a complex mixture of substances, in any physical state or in a combination of physical states.

To “derive” a profile data set from a sample includes determining a set of values associated with constituents of a Gene Expression Panel either (i) by direct measurement of such constituents in a biological sample or (ii) by measurement of such constituents in a second biological sample that has been exposed to the original sample or to matter derived from the original sample.

“Distinct RNA or protein constituent” in a panel of constituents is a distinct expressed product of a gene, whether RNA or protein. An “expression” product of a gene includes the gene product whether RNA or protein resulting from translation of the messenger RNA.

A “Gene Expression Panel” is an experimentally verified set of constituents, each constituent being a distinct expressed product of a gene, whether RNA or protein, wherein constituents of the set are selected so that their measurement provides a measurement of a targeted biological condition.

A “Gene Expression Profile” is a set of values associated with constituents of a Gene Expression Panel resulting from evaluation of a biological sample (or population or set of samples).

A “Gene Expression Profile Inflammatory index” is the value of an index function that provides a mapping from an instance of a Gene Expression Profile into a single-valued measure of inflammatory condition.

The “health” of a subject includes mental, emotional, physical, spiritual, allopathic, naturopathic and homeopathic condition of the subject.

“Index” is an arithmetically or mathematically derived numerical characteristic developed for aid in simplifying or disclosing or informing the analysis of more complex quantitative information. A disease or population index may be determined by the application of a specific algorithm to a plurality of subjects or samples with a common biological condition.

“Inflammation” is used herein in the general medical sense of the word and may be an acute or chronic; simple or suppurative; localized or disseminated; cellular and tissue response, initiated or sustained by any number of chemical, physical or biological agents or combination of agents.

“Inflammatory state” is used to indicate the relative biological condition of a subject resulting from inflammation, or characterizing the degree of inflammation

A “large number” of data sets based on a common panel of genes is a number of data sets sufficiently large to permit a statistically significant conclusion to be drawn with respect to an instance of a data set based on the same panel.

A “normative” condition of a subject to whom a composition is to be administered means the condition of a subject before administration, even if the subject happens to be suffering from a disease.

A “panel” of genes is a set of genes including at least two constituents.

A “sample” from a subject may include a single cell or multiple cells or fragments of cells or an aliquot of body fluid, taken from the subject, by means including venipuncture, excretion, ejaculation, massage, biopsy, needle aspirate, lavage sample, scraping, surgical incision or intervention or other means known in the art.

A “Signature Profile” is an experimentally verified subset of a Gene Expression Profile selected to discriminate a biological condition, agent or physiological mechanism of action.

A “Signature Panel” is a subset of a Gene Expression Panel, the constituents of which are selected to permit discrimination of a biological condition, agent or physiological mechanism of action.

A “subject” is a cell, tissue, or organism, human or non-human, whether in vivo, ex vivo or in vitro, under observation. When we refer to evaluating the biological condition of a subject based on a sample from the subject, we include using blood or other tissue sample from a human subject to evaluate the human subject's condition; but we also include, for example, using a blood sample itself as the subject to evaluate, for example, the effect of therapy or an agent upon the sample.

A “stimulus” includes (i) a monitored physical interaction with a subject, for example ultraviolet A or B, or light therapy for seasonal affective disorder, or treatment of psoriasis with psoralen or treatment of melanoma with embedded radioactive seeds, other radiation exposure, and (ii) any monitored physical, mental, emotional, or spiritual activity or inactivity of a subject.

“Therapy” includes all interventions whether biological, chemical, physical, metaphysical, or combination of the foregoing, intended to sustain or alter the monitored biological condition of a subject.

The PCT patent application publication number WO 01/25473, published Apr. 12, 2001, entitled “Systems and Methods for Characterizing a Biological Condition or Agent Using Calibrated Gene Expression Profiles,” filed for an invention by inventors herein, and which is herein incorporated by reference, discloses the use of Gene Expression Panels for the evaluation of (i) biological condition (including with respect to health and disease) and (ii) the effect of one or more agents on biological condition (including with respect to health, toxicity, therapeutic treatment and drug interaction).

In particular, Gene Expression Panels may be used for measurement of therapeutic efficacy of natural or synthetic compositions or stimuli that may be formulated individually or in combinations or mixtures for a range of targeted biological conditions; prediction of toxicological effects and dose effectiveness of a composition or mixture of compositions for an individual or for a population or set of individuals or for a population of cells; determination of how two or more different agents administered in a single treatment might interact so as to detect any of synergistic, additive, negative, neutral or toxic activity; performing pre-clinical and clinical trials by providing new criteria for pre-selecting subjects according to informative profile data sets for revealing disease status; and conducting preliminary dosage studies for these patients prior to conducting phase 1 or 2 trials. These Gene Expression Panels may be employed with respect to samples derived from subjects in order to evaluate their biological condition.

A Gene Expression Panel is selected in a manner so that quantitative measurement of RNA or protein constituents in the Panel constitutes a measurement of a biological condition of a subject. In one kind of arrangement, a calibrated profile data set is employed. Each member of the calibrated profile data set is a function of (i) a measure of a distinct constituent of a Gene Expression Panel and (ii) a baseline quantity.

We have found that valuable and unexpected results may be achieved when the quantitative measurement of constituents is performed under repeatable conditions (within a degree of repeatability of measurement of better than twenty percent, and preferably five percent or better, and more preferably three percent or better). For the purposes of this description and the following claims, we regard a degree of repeatability of measurement of better than twenty percent as providing measurement conditions that are “substantially repeatable”. In particular, it is desirable that, each time a measurement is obtained corresponding to the level of expression of a constituent in a particular sample, substantially the same measurement should result for the substantially the same level of expression. In this manner, expression levels for a constituent in a Gene Expression Panel may be meaningfully compared from sample to sample. Even if the expression level measurements for a particular constituent are inaccurate (for example, say, 30% too low), the criterion of repeatability means that all measurements for this constituent, if skewed, will nevertheless be skewed systematically, and therefore measurements of expression level of the constituent may be compared meaningfully. In this fashion valuable information may be obtained and compared concerning expression of the constituent under varied circumstances.

In addition to the criterion of repeatability, it is desirable that a second criterion also be satisfied, namely that quantitative measurement of constituents is performed under conditions wherein efficiencies of amplification for all constituents are substantially similar (within one to two percent and typically one percent or less). When both of these criteria are satisfied, then measurement of the expression level of one constituent may be meaningfully compared with measurement of the expression level of another constituent in a given sample and from sample to sample.

Present embodiments relate to the use of an index or algorithm resulting from quantitative measurement of constituents, and optionally in addition, derived from either expert analysis or computational biology (a) in the analysis of complex data sets; (b) to control or normalize the influence of uninformative or otherwise minor variances in gene expression values between samples or subjects; (c) to simplify the characterization of a complex data set for comparison to other complex data sets, databases or indices or algorithms derived from complex data sets; (d) to monitor a biological condition of a subject; (e) for measurement of therapeutic efficacy of natural or synthetic compositions or stimuli that may be formulated individually or in combinations or mixtures for a range of targeted biological conditions; (f) for predictions of toxicological effects and dose effectiveness of a composition or mixture of compositions for an individual or for a population or set of individuals or for a population of cells; (g) for determination of how two or more different agents administered in a single treatment might interact so as to detect any of synergistic, additive, negative, neutral of toxic activity (h) for performing pre-clinical and clinical trials by providing new criteria for pre-selecting subjects according to informative profile data sets for revealing disease status and conducting preliminary dosage studies for these patients prior to conducting phase 1 or 2 trials.

Gene expression profiling and the use of index characterization for a particular condition or agent or both may be used to reduce the cost of phase 3 clinical trials and may be used beyond phase 3 trials; labeling for approved drugs; selection of suitable medication in a class of medications for a particular patient that is directed to their unique physiology; diagnosing or determining a prognosis of a medical condition or an infection which may precede onset of symptoms or alternatively diagnosing adverse side effects associated with administration of a therapeutic agent; managing the health care of a patient; and quality control for different batches of an agent or a mixture of agents.

### The subject

The methods disclosed here may be applied to cells of humans, mammals or other organisms without the need for undue experimentation by one of ordinary skill in the art because all cells transcribe RNA and it is known in the art how to extract RNA from all types of cells.

### Selecting Constituents of a Gene Expression Panel

The general approach to selecting constituents of a Gene Expression Panel has been described in PCT application publication number WO 01/25473. We have designed and experimentally verified a wide range of Gene Expression Panels, each panel providing a quantitative measure, of biological condition, that is derived from a sample of blood or other tissue. For each panel, experiments have verified that a Gene Expression Profile using the panel's constituents is informative of a biological condition. (We show elsewhere that in being informative of biological condition, the Gene Expression Profile can be used to used, among other things, to measure the effectiveness of therapy, as well as to provide a target for therapeutic intervention.) Examples of Gene Expression Panels, along with a brief description of each panel constituent, are provided in tables attached hereto as follows:

Table 1. Inflammation Gene Expression Panel

Table 2. Diabetes Gene Expression Panel

Table 3. Prostate Gene Expression Panel

Table 4. Skin Response Gene Expression Panel

Table 5. Liver Metabolism and Disease Gene Expression Panel

Table 6. Endothelial Gene Expression Panel

Table 7. Cell Health and Apoptosis Gene Expression Panel

Table 8. Cytokine Gene Expression Panel

Table 9. TNF/IL1 Inhibition Gene Expression Panel

Table 10. Chemokine Gene Expression Panel

Table 11. Breast Cancer Gene Expression Panel

Table 12. Infectious Disease Gene Expression Panel

Other panels may be constructed and experimentally verified by one of ordinary skill in the art in accordance with the principles articulated in the present application.

### Design of Assays

We commonly run a sample through a panel in quadruplicate; that is, a sample is divided into aliquots and for each aliquot we measure concentrations of each constituent in a Gene Expression Panel. Over a total of 900 constituent assays, with each assay conducted in quadruplicate, we found an average coefficient of variation, (standard deviation/average)*100, of less than 2 percent, typically less than 1 percent, among results for each assay. This figure is a measure of what we call “intra-assay variability”. We have also conducted assays on different occasions using the same sample material. With 72 assays, resulting from concentration measurements of constituents in a panel of 24 members, and such concentration measurements determined on three different occasions over time, we found an average coefficient of variation of less than 5 percent, typically less than 2 percent. We regard this as a measure of what we call “inter-assay variability”.

We have found it valuable in using the quadruplicate test results to identify and eliminate data points that are statistical “outliers”; such data points are those that differ by a percentage greater, for example, than 3% of the average of all four values and that do not result from any systematic skew that is greater, for example, than 1%. Moreover, if more than one data point in a set of four is excluded by this procedure, then all data for the relevant constituent is discarded.

### Measurement of Gene Expression for a Constituent in the Panel

For measuring the amount of a particular RNA in a sample, we have used methods known to one of ordinary skill in the art to extract and quantify transcribed RNA from a sample with respect to a constituent of a Gene Expression Panel. (See detailed protocols below. Also see PCT application publication number WO 98/24935 herein incorporated by reference for RNA analysis protocols). Briefly, RNA is extracted from a sample such as a tissue, body fluid, or culture medium in which a population of cells of a subject might be growing. For example, cells may be lysed and RNA eluted in a suitable solution in which to conduct a DNAse reaction. First strand synthesis may be performed using a reverse transcriptase. Gene amplification, more specifically quantitative PCR assays, can then conducted and the gene of interest size calibrated against a marker such as 18S rRNA (Hirayama et al., Blood 92, 1998: 46-52). Samples are measured in multiple duplicates, for example, 4 replicates. Relative quantitation of the mRNA is determined by the difference in threshhold cycles between the intemal control and the gene of interest. In an embodiment of the invention, quantitative PCR is performed using amplification, reporting agents and instruments such as those supplied commercially by Applied Biosystems (Foster City, Calif.). Given a defined efficiency of amplification of target transcripts, the point (e.g., cycle number) that signal from amplified target template is detectable may be directly related to the amount of specific message transcript in the measured sample. Similarly, other quantifiable signals such as fluorescence, enzyme activity, disintegrations per minute, absorbance, etc., when correlated to a known concentration of target templates (e.g., a reference standard curve) or normalized to a standard with limited variability can be used to quantify the number of target templates in an unknown sample.

Although not limited to amplification methods, quantitative gene expression techniques may utilize amplification of the target transcript. Alternatively or in combination with amplification of the target transcript, amplification of the reporter signal may also be used. Amplification of the target template may be accomplished by isothermic gene amplification strategies, or by gene amplification by thermal cycling such as PCR.

It is desirable to obtain a definable and reproducible correlation between the amplified target or reporter and the concentration of starting templates. We have discovered that this objective can be achieved by careful attention to, for example, consistent primer-template ratios and a strict adherence to a narrow permissible level of experimental amplification efficiencies (for example 99.0 to 100% relative efficiency, typically 99.8 to 100% relative efficiency). For example, in determining gene expression levels with regard to a single Gene Expression Profile, it is necessary that all constituents of the panels maintain a similar and limited range of primer template ratios (for example, within a 10-fold range) and amplification efficiencies (within, for example, less than 1%) to permit accurate and precise relative measurements for each constituent. We regard amplification efficiencies as being “substantially similar”, for the purposes of this description and the following claims, if they differ by no more than approximately 10%. Preferably they should differ by less than approximately 2% and more preferably by less than approximately 1%. These constraints should be observed over the entire range of concentration levels to be measured associated with the relevant biological condition. While it is thus necessary for various embodiments herein to satisfy criteria that measurements are achieved under measurement conditions that are substantially repeatable and wherein specificity and efficiencies of amplification for all constituents are substantially similar, nevertheless, it is within the scope of the present invention as claimed herein to achieve such measurement conditions by adjusting assay results that do not satisfy these criteria directly, in such a manner as to compensate for errors, so that the criteria are satisfied after suitable adjustment of assay results.

In practice, we run tests to assure that these conditions are satisfied. For example, we typically design and manufacture a number of primer-probe sets, and determine experimentally which set gives the best performance. Even though primer-probe design and manufacture can be enhanced using computer techniques known in the art, and notwithstanding common practice, we still find that experimental validation is useful. Moreover, in the course of experimental validation, we associate with the selected primer-probe combination a set of features:

The reverse primer should be complementary to the coding DNA strand. In one embodiment, the primer should be located across an intron-exon junction, with not more than three bases of the three-prime end of the reverse primer complementary to the proximal exon. (If more than three bases are complementary, then it would tend to competitively amplify genomic DNA.)

In an embodiment of the invention, the primer probe should amplify cDNA of less than 110 bases in length and should not amplify genomic DNA or transcripts or cDNA from related but biologically irrelevant loci.

A suitable target of the selected primer probe is first strand cDNA, which may be prepared, in one embodiment, is described as follows:

(a) Use of Whole Blood for Ex Vivo Assessment of a Biological Condition Affected by an Agent.

Human blood is obtained by venipuncture and prepared for assay by separating samples for baseline, no stimulus, and stimulus with sufficient volume for at least three time points. Typical stimuli include lipopolysaccharide (LPS), phytohemagglutinin (PHA) and heat-killed staphylococci (HKS) or carrageean and may be used individually (typically) or in combination. The aliquots of heparinized, whole blood are mixed without stimulus and held at 37° C. in an atmosphere of 5% CO2 for 30 minutes. Stimulus is added at varying concentrations, mixed and held loosely capped at 37° C. for 30 min. Additional test compounds may be added at this point and held for varying times depending on the expected pharmacokinetics of the test compound. At defined times, cells are collected by centrifugation, the plasma removed and RNA extracted by various standard means.

Nucleic acids, RNA and or DNA are purified from cells, tissues or fluids of the test population of cells or indicator cell lines. RNA is preferentially obtained from the nucleic acid mix using a variety of standard procedures (or RNA Isolation Strategies, pp. 55-104, in RNA Methodologies, A laboratory guide for isolation and characterization, 2nd edition, 1998, Robert E. Farrell, Jr., Ed., Academic Press), in the present using a filter-based RNA isolation system from Ambion (RNAqueous™, Phenol-free Total RNA Isolation Kit. Catalog #1912, version 9908; Austin, Tex.).

In accordance with one procedure, the whole blood assay for Gene Expression Profiles determination was carried out as follows: Human whole blood was drawn into 10 mL Vacutainer tubes with Sodium Heparin. Blood samples were mixed by gently inverting tubes 4-5 times. The blood was used within 10-15 minutes of draw. In the experiments, blood was diluted 2-fold, i.e. per sample per time point, 0.6 mL whole blood+0.6 mL stimulus. The assay medium was prepared and the stimulus added as appropriate.

A quantity (0.6 mL) of whole blood was then added into each 12×75 mm polypropylene tube, 0.6 mL of 2×LPS (from E. coli serotye 0127:B8, Sigma#L3880 or serotype 055, Sigma #L4005, 10 ng/ml, subject to change in different lots) into LPS tubes was added. Next, 0.6 mL assay medium was added to the “control” tubes with duplicate tubes for each condition. The caps were closed tightly. The tubes were inverted 2-3 times to mix samples. Caps were loosened to first stop and the tubes incubated @37° C., 5% CO2 for 6 hours. At 6 hours, samples were gently mixed to resuspend blood cells, and 1 mL was removed from each tube (using a micropipettor with barrier tip), and transferred to a 2 mL “dolphin” microfuge tube (Costar #3213).

The samples were then centrifuged for 5 min at 500×g, ambient temperature (IEC centrifuge or equivalent, in microfuge tube adapters in swinging bucket), and as much serum from each tube was removed as possible and discarded. Cell pellets were placed on ice; and RNA extracted as soon as possible using an Ambion RNAqueous kit.

(b) Amplification Strategies.

Specific RNAs are amplified using message specific primers or random primers. The specific primers are synthesized from data obtained from public databases (e.g., Unigene, National Center for Biotechnology Information, National Library of Medicine, Bethesda, Md.), including information from genomic and cDNA libraries obtained from humans and other animals. Primers are chosen to preferentially amplify from specific RNAs obtained from the test or indicator samples, see, for example, RT PCR, Chapter 15 in RNA Methodologies, A laboratory guide for isolation and characterization, 2nd edition, 1998, Robert E. Farrell, Jr., Ed., Academic Press; or Chapter 22 pp. 143-151, RNA isolation and characterization protocols, Methods in molecular biology, Volume 86, 1998, R. Rapley and D. L. Manning Eds., Human Press, or 14 in Statistical refinement of primer design parameters, Chapter 5, pp. 55-72, PCR applications: protocols for functional genomics, M. A. Innis, D. H. Gelfand and J. J. Sninsky, Eds., 1999, Academic Press). Amplifications are carried out in either isothermic conditions or using a thermal cycler (for example, a ABI 9600 or 9700 or 7700 obtained from Applied Biosystems, Foster City, Calif.; see Nucleic acid detection methods, pp. 1-24, in Molecular methods for virus detection, D. L. Wiedbrauk and D. H., Farkas, Eds., 1995, Academic Press). Amplified nucleic acids are detected using fluorescent-tagged detection primers (see, for example, Taqman™ PCR Reagent Kit, Protocol, part number 402823 revision A, 1996, Applied Biosystems, Foster City Calif.) that are identified and synthesized from publicly known databases as described for the amplification primers. In the present case, amplified DNA is detected and quantified using the ABI Prism 7700 Sequence Detection System obtained from Applied Biosystems (Foster City, Calif.). Amounts of specific RNAs contained in the test sample or obtained from the indicator cell lines can be related to the relative quantity of fluorescence observed (see for example, Advances in quantitative PCR technology: 5′ nuclease assays, Y. S. Lie and CJ. Petropolus, Current Opinion in Biotechnology, 1998, 9:43-48, or Rapid thermal cycling and PCR kinetics, pp. 211-229, chapter 14 in PCR applications: protocols for functional genomics, M. A. Innis, D. H. Gelfand and J. J. Sninsky, Eds., 1999, Academic Press).

As a particular implementation of the approach described here, we describe in detail a procedure for synthesis of first strand cDNA for use in PCR. This procedure can be used for both whole blood RNA and RNA extracted from cultured cells (i.e. THP-1 cells).

### Materials

1. Applied Biosystems TAQMAN Reverse Transcription Reagents Kit (P/N 808-0234). Kit Components: 10× TaqMan RT Buffer, 25 mM Magnesium chloride, deoxyNTPs mixture, Random Hexamers, RNase Inhibitor, MultiScribe Reverse Transcriptase (50 U/mL) (2) RNase/DNase free water (DEPC Treated Water from Ambion (P/N 9915G), or equivalent)

### Methods

1. Place RNase Inhibitor and MultiScribe Reverse Transcriptase on ice immediately. All other reagents can be thawed at room temperature and then placed on ice.

2. Remove RNA samples from −80° C. freezer and thaw at room temperature and then place immediately on ice.

3. Prepare the following cocktail of Reverse Transcriptase Reagents for each 100 mL RT reaction (for multiple samples, prepare extra cocktail to allow for pipetting error):

| 1 reaction (mL) | 11X, e.g. 10 samples (mL) | |
|---|---|---|
| 10X RT Buffer | 10.0 | 110.0 |
| 25 mM MgCl2 | 22.0 | 242.0 |
| dNTPs | 20.0 | 220.0 |
| Random Hexamers | 5.0 | 55.0 |
| RNAse Inhibitor | 2.0 | 22.0 |
| Reverse Transcriptase | 2.5 | 27.5 |
| Water | 18.5 | 203.5 |
| Total: | 80.0 | 880.0 (80 mL per sample) |

4. Bring each RNA sample to a total volume of 20 mL in a 1.5 mL microcentrifuge tube (for example, for THP-1 RNA, remove 10 mL RNA and dilute to 20 mL with RNase/DNase free water, for whole blood RNA use 20 mL total RNA) and add 80 mL RT reaction mix from step 5,2,3. Mix by pipetting up and down.

5. Incubate sample at room temperature for 10 minutes.

6. Incubate sample at 37° C. for 1 hour.

7. Incubate sample at 90° C. for 10 minutes.

8. Quick spin samples in microcentrifuge.

9. Place sample on ice if doing PCR immediately, otherwise store sample at −20° C. for future use.

10. PCR QC should be run on all RT samples using 18S and b-actin (see SOP 200-020).

The use of the primer probe with the first strand cDNA as described above to permit measurement of constituents of a Gene Expression Panel is as follows:

Set up of a 24-gene Human Gene Expression Panel for Inflammation.

### Materials

1. 20® Primer/Probe Mix for each gene of interest.

2. 20® Primer/Probe Mix for 18S endogenous control.

3. 2® Taqman Universal PCR Master Mix.

4. cDNA transcribed from RNA extracted from cells.

5. Applied Biosystems 96-Well Optical Reaction Plates.

6. Applied Biosystems Optical Caps, or optical-clear film.

7. Applied Biosystem Prism 7700 Sequence Detector.

### Methods

1. Make stocks of each Primer/Probe mix containing the Primer/Probe for the gene of interest, Primer/Probe for 18S endogenous control, and 2® PCR Master Mix as follows. Make sufficient excess to allow for pipetting error e.g. approximately 10% excess. The following example illustrates a typical set up for one gene with quadruplicate samples testing two conditions (2 plates).

| | | 9X |
|---|---|---|
| | 1X (1 well) | (2 plates worth) |
| 2X Master Mix | 12.50 | 112.50 |
| 20X 18S Primer/Probe Mix | 1.25 | 11.25 |
| 20X Gene of interest Primer/Probe Mix | 1.25 | 11.25 |
| Total | 15.00 | 135.00 |

2. Make stocks of cDNA targets by diluting 95 μl of cDNA into 2000 μl of water. The amount of cDNA is adjusted to give Ct values between 10 and 18, typically between 12 and 13.

3. Pipette 15 μl of Primer/Probe mix into the appropriate wells of an Applied Biosystems 96-Well Optical Reaction Plate.

4. Pipette 10 μl of cDNA stock solution into each well of the Applied Biosystems 96-Well Optical Reaction Plate.

5. Seal the plate with Applied Biosystems Optical Caps, or optical-clear film.

6. Analyze the plate on the AB Prism 7700 Sequence Detector.

Methods herein may also be applied using proteins where sensitive quantitative techniques, such as an Enzyme Linked ImmunoSorbent Assay (ELISA) or mass spectroscopy, are available and well-known in the art for measuring the amount of a protein constituent. (see WO 98/24935 herein incorporated by reference).

### Baseline Profile Data Sets

The analyses of samples from single individuals and from large groups of individuals provide a library of profile data sets relating to a particular panel or series of panels. These profile data sets may be stored as records in a library for use as baseline profile data sets. As the term “baseline” suggests, the stored baseline profile data sets serve as comparators for providing a calibrated profile data set that is informative about a biological condition or agent. Baseline profile data sets may be stored in libraries and classified in a number of cross-referential ways. One form of classification may rely on the characteristics of the panels from which the data sets are derived. Another form of classification may be by particular biological condition. The concept of biological condition encompasses any state in which a cell or population of cells may be found at any one time. This state may reflect geography of samples, sex of subjects or any other discriminator. Some of the discriminators may overlap. The libraries may also be accessed for records associated with a single subject or particular clinical trial. The classification of baseline profile data sets may further be annotated with medical information about a particular subject, a medical condition, a particular agent etc.

The choice of a baseline profile data set for creating a calibrated profile data set is related to the biological condition to be evaluated, monitored, or predicted, as well as, the intended use of the calibrated panel, e.g., as to monitor drug development, quality control or other uses. It may be desirable to access baseline profile data sets from the same subject for whom a first profile data set is obtained or from different subject at varying times, exposures to stimuli, drugs or complex compounds; or may be derived from like or dissimilar populations or sets of subjects.

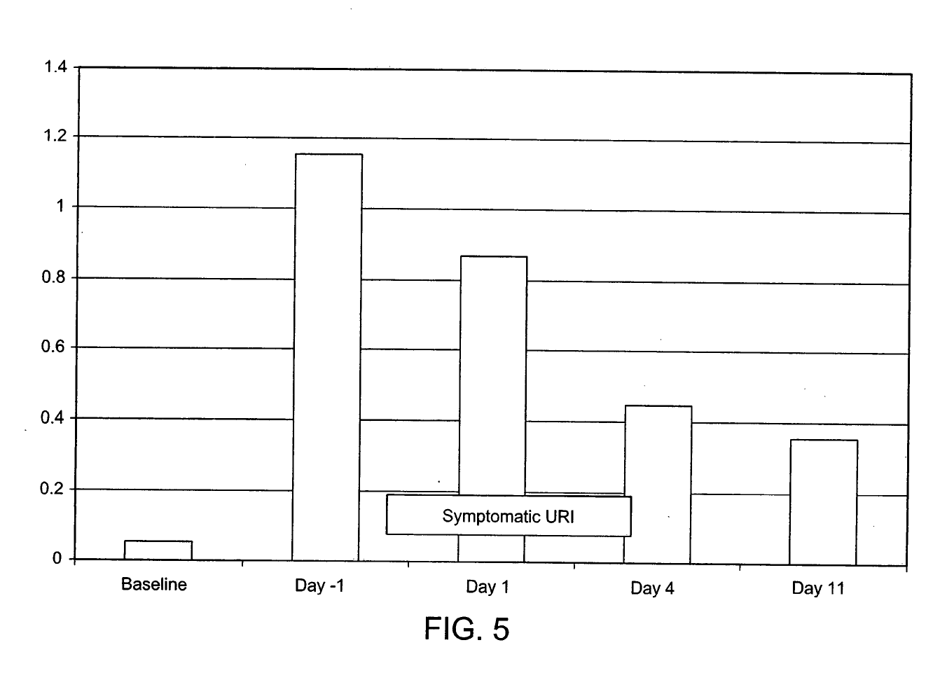

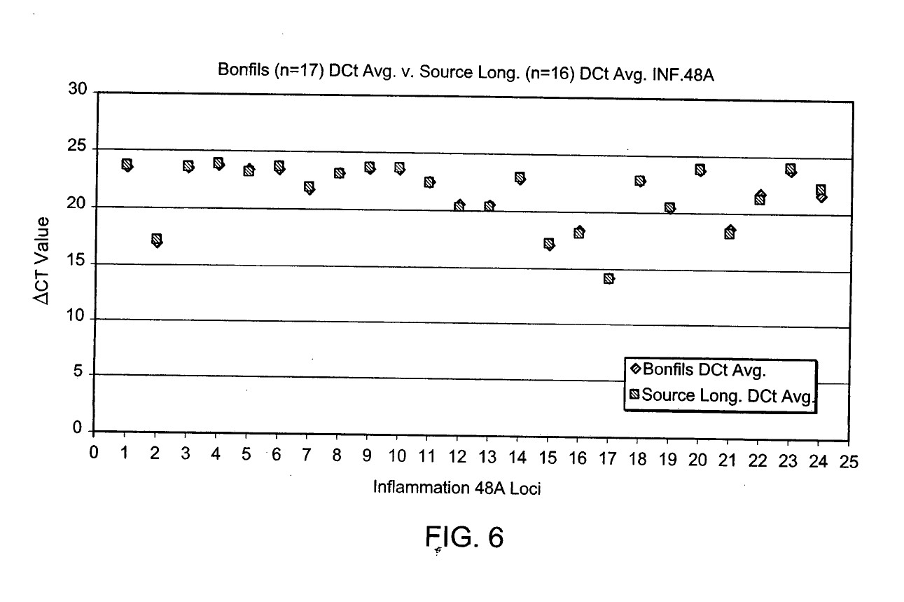

The profile data set may arise from the same subject for which the first data set is obtained, where the sample is taken at a separate or similar time, a different or similar site or in a different or similar biological condition. For example, FIG. 5 provides a protocol in which the sample is taken before stimulation or after stimulation. The profile data set obtained from the unstimulated sample may serve as a baseline profile data set for the sample taken after stimulation. The baseline data set may also be derived from a library containing profile data sets of a population or set of subjects having some defining characteristic or biological condition. The baseline profile data set may also correspond to some ex vivo or in vitro properties associated with an in vitro cell culture. The resultant calibrated profile data sets may then be stored as a record in a database or library (FIG. 6) along with or separate from the baseline profile data base and optionally the first profile data set although the first profile data set would normally become incorporated into a baseline profile data set under suitable classification criteria. The remarkable consistency of Gene Expression Profiles associated with a given biological condition makes it valuable to store profile data, which can be used, among other things for normative reference purposes. The normative reference can serve to indicate the degree to which a subject conforms to a given biological condition (healthy or diseased) and, alternatively or in addition, to provide a target for clinical intervention.

Selected baseline profile data sets may be also be used as a standard by which to judge manufacturing lots in terms of efficacy, toxicity, etc. Where the effect of a therapeutic agent is being measured, the baseline data set may correspond to Gene Expression Profiles taken before administration of the agent. Where quality control for a newly manufactured product is being determined, the baseline data set may correspond with a gold standard for that product. However, any suitable normalization techniques may be employed. For example, an average baseline profile data set is obtained from authentic material of a naturally grown herbal nutriceutical and compared over time and over different lots in order to demonstrate consistency, or lack of consistency, in lots of compounds prepared for release.

### Calibrated Data

Given the repeatability we have achieved in measurement of gene expression, described above in connection with “Gene Expression Panels” and “gene amplification”, we conclude that where differences occur in measurement under such conditions, the differences are attributable to differences in biological condition. Thus we have found that calibrated profile data sets are highly reproducible in samples taken from the same individual under the same conditions. We have similarly found that calibrated profile data sets are reproducible in samples that are repeatedly tested. We have also found repeated instances wherein calibrated profile data sets obtained when samples from a subject are exposed ex vivo to a compound are comparable to calibrated profile data from a sample that has been exposed to a sample in vivo. We have also found, importantly, that an indicator cell line treated with an agent can in many cases provide calibrated profile data sets comparable to those obtained from in vivo or ex vivo populations of cells. Moreover, we have found that administering a sample from a subject onto indicator cells can provide informative calibrated profile data sets with respect to the biological condition of the subject including the health, disease states, therapeutic interventions, aging or exposure to environmental stimuli or toxins of the subject.

### Calculation of Calibrated Profile Data Sets and Computational Aids

The calibrated profile data set may be expressed in a spreadsheet or represented graphically for example, in a bar chart or tabular form but may also be expressed in a three dimensional representation. The function relating the baseline and profile data may be a ratio expressed as a logarithm. The constituent may be itemized on the x-axis and the logarithmic scale may be on the y-axis. Members of a calibrated data set may be expressed as a positive value representing a relative enhancement of gene expression or as a negative value representing a relative reduction in gene expression with respect to the baseline.

Each member of the calibrated profile data set should be reproducible within a range with respect to similar samples taken from the subject under similar conditions. For example, the calibrated profile data sets may be reproducible within one order of magnitude with respect to similar samples taken from the subject under similar conditions. More particularly, the members may be reproducible within 50%, more particularly reproducible within 20%, and typically within 10%. In accordance with embodiments of the invention, a pattern of increasing, decreasing and no change in relative gene expression from each of a plurality of gene loci examined in the Gene Expression Panel may be used to prepare a calibrated profile set that is informative with regards to a biological condition, biological efficacy of an agent treatment conditions or for comparison to populations or sets of subjects or samples, or for comparison to populations of cells. Patterns of this nature may be used to identify likely candidates for a drug trial, used alone or in combination with other clinical indicators to be diagnostic or prognostic with respect to a biological condition or may be used to guide the development of a pharmaceutical or nutriceutical through manufacture, testing and marketing.

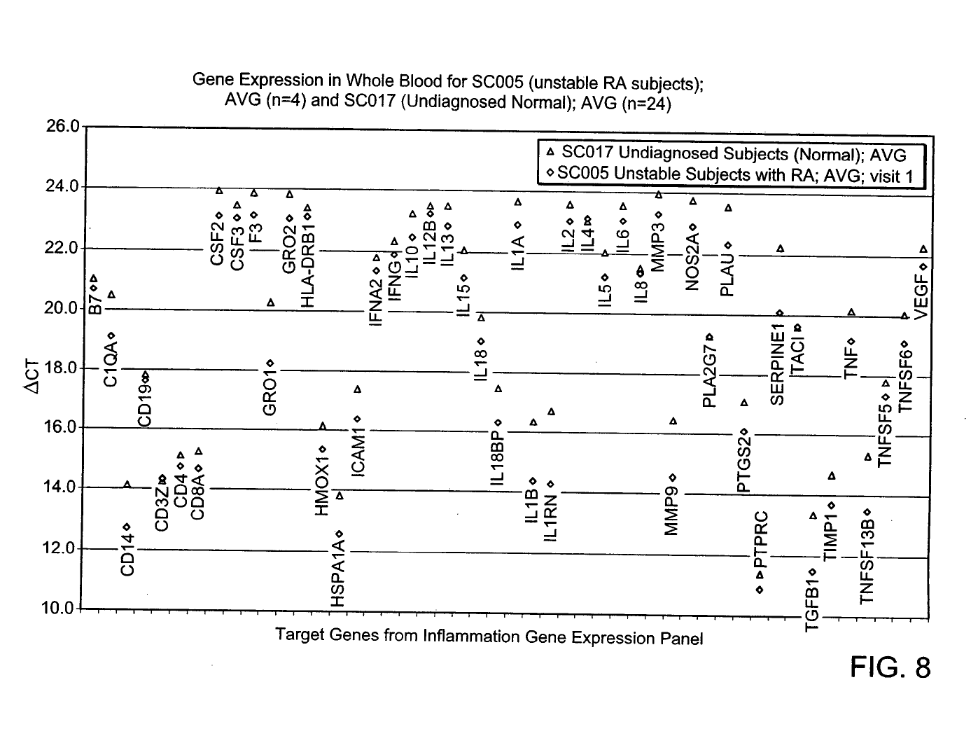

The numerical data obtained from quantitative gene expression and numerical data from calibrated gene expression relative to a baseline profile data set may be stored in databases or digital storage mediums and may retrieved for purposes including managing patient health care or for conducting clinical trials or for characterizing a drug. The data may be transferred in physical or wireless networks via the World Wide Web, email, or internet access site for example or by hard copy so as to be collected and pooled from distant geographic sites (FIG. 8).

In an embodiment of the present invention, a descriptive record is stored in a single database or multiple databases where the stored data includes the raw gene expression data (first profile data set) prior to transformation by use of a baseline profile data set, as well as a record of the baseline profile data set used to generate the calibrated profile data set including for example, annotations regarding whether the baseline profile data set is derived from a particular Signature Panel and any other annotation that facilitates interpretation and use of the data.

Because the data is in a universal format, data handling may readily be done with a computer. The data is organized so as to provide an output optionally corresponding to a graphical representation of a calibrated data set.

For example, a distinct sample derived from a subject being at least one of RNA or protein may be denoted as Pₗ. The first profile data set derived from sample Pₗ is denoted Mⱼ, where Mⱼ is a quantitative measure of a distinct RNA or protein constituent of Pₗ. The record Ri is a ratio of M and P and may be annotated with additional data on the subject relating to, for example, age, diet, ethnicity, gender, geographic location, medical disorder, mental disorder, medication, physical activity, body mass and environmental exposure. Moreover, data handling may further include accessing data from a second condition database which may contain additional medical data not presently held with the calibrated profile data sets. In this context, data access may be via a computer network.

The above described data storage on a computer may provide the information in a form that can be accessed by a user. Accordingly, the user may load the information onto a second access site including downloading the information. However, access may be restricted to users having a password or other security device so as to protect the medical records contained within. A feature of this embodiment of the invention is the ability of a user to add new or annotated records to the data set so the records become part of the biological information.

The graphical representation of calibrated profile data sets pertaining to a product such as a drug provides an opportunity for standardizing a product by means of the calibrated profile, more particularly a signature profile. The profile may be used as a feature with which to demonstrate relative efficacy, differences in mechanisms of actions, etc. compared to other drugs approved for similar or different uses.

The various embodiments of the invention may be also implemented as a computer program product for use with a computer system. The product may include program code for deriving a first profile data set and for producing calibrated profiles. Such implementation may include a series of computer instructions fixed either on a tangible medium, such as a computer readable medium (for example, a diskette, CD-ROM, ROM, or fixed disk), or transmittable to a computer system via a modem or other interface device, such as a communications adapter coupled to a network. The network coupling may be for example, over optical or wired communications lines or via wireless techniques (for example, microwave, infrared or other transmission techniques) or some combination of these. The series of computer instructions preferably embodies all or part of the functionality previously described herein with respect to the system. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (for example, shrink wrapped software), preloaded with a computer system (for example, on system ROM or fixed disk), or distributed from a server or electronic bulletin board over a network (for example, the Internet or World Wide Web). In addition, a computer system is further provided including derivative modules for deriving a first data set and a calibration profile data set.

The calibration profile data sets in graphical or tabular form, the associated databases, and the calculated index or derived algorithm, together with information extracted from the panels, the databases, the data sets or the indices or algorithms are commodities that can be sold together or separately for a variety of purposes as described in WO 01/25473.

### Index construction

In combination, (i) the remarkable consistency of Gene Expression Profiles with respect to a biological condition across a population or set of subject or samples, or across a population of cells and (ii) the use of procedures that provide substantially reproducible measurement of constituents in a Gene Expression Panel giving rise to a Gene Expression Profile, under measurement conditions wherein specificity and efficiencies of amplification for all constituents of the panel are substantially similar, make possible the use of an index that characterizes a Gene Expression Profile, and which therefore provides a measurement of a biological condition.

An index may be constructed using an index function that maps values in a Gene Expression Profile into a single value that is pertinent to the biological condition at hand. The values in a Gene Expression Profile are the amounts of each constituent of the Gene Expression Panel that corresponds to the Gene Expression Profile. These constituent amounts form a profile data set, and the index function generates a single value—the index—from the members of the profile data set.

The index function may conveniently be constructed as a linear sum of terms, each term being what we call a “contribution function” of a member of the profile data set. For example, the contribution function may be a constant times a power of a member of the profile data set. So the index function would have the form

I=ΣCᵢMᵢ^{P(i)},

where I is the index, Mᵢ is the value of the member i of the profile data set, Cᵢ is a constant, and P(i) is a power to which Mᵢ is raised, the sum being formed for all integral values of i up to the number of members in the data set. We thus have a linear polynomial expression.

The values Cᵢ and P(i) may be determined in a number of ways, so that the index I is informative of the pertinent biological condition. One way is to apply statistical techniques, such as latent class modeling, to the profile data sets to correlate clinical data or experimentally derived data, or other data pertinent to the biological condition. In this connection, for example, may be employed the software from Statistical Innovations, Belmont, Mass., called Latent Gold®. See the web pages at www.statisticalinnovations.com/lg/, which are hereby incorporated herein by reference.

Alternatively, other simpler modeling techniques may be employed in a manner known in the art. The index function for inflammation may be constructed, for example, in a manner that a greater degree of inflammation (as determined by the a profile data set for the Inflammation Gene Expression Profile) correlates with a large value of the index function. In a simple embodiment, therefore, each P(i) may be +1 or −1, depending on whether the constituent increases or decreases with increasing inflammation. As discussed in further detail below, we have constructed a meaningful inflammation index that is proportional to the expression

¼{IL1A}+¼{IL1B}+¼{TNF}+¼{INFG}−1/{IL10},

where the braces around a constituent designate measurement of such constituent and the constituents are a subset of the Inflammation Gene Expression Panel of Table 1.

Just as a baseline profile data set, discussed above, can be used to provide an appropriate normative reference, and can even be used to create a Calibrated profile data set, as discussed above, based on the normative reference, an index that characterizes a Gene Expression Profile can also be provided with a normative value of the index function used to create the index. This normative value can be determined with respect to a relevant population or set of subjects or samples or to a relevant population of cells, so that the index may be interpreted in relation to the normative value. The relevant population or set of subjects or samples, or relevant population of cells may have in common a property that is at least one of age range, gender, ethnicity, geographic location, nutritional history, medical condition, clinical indicator, medication, physical activity, body mass, and environmental exposure.

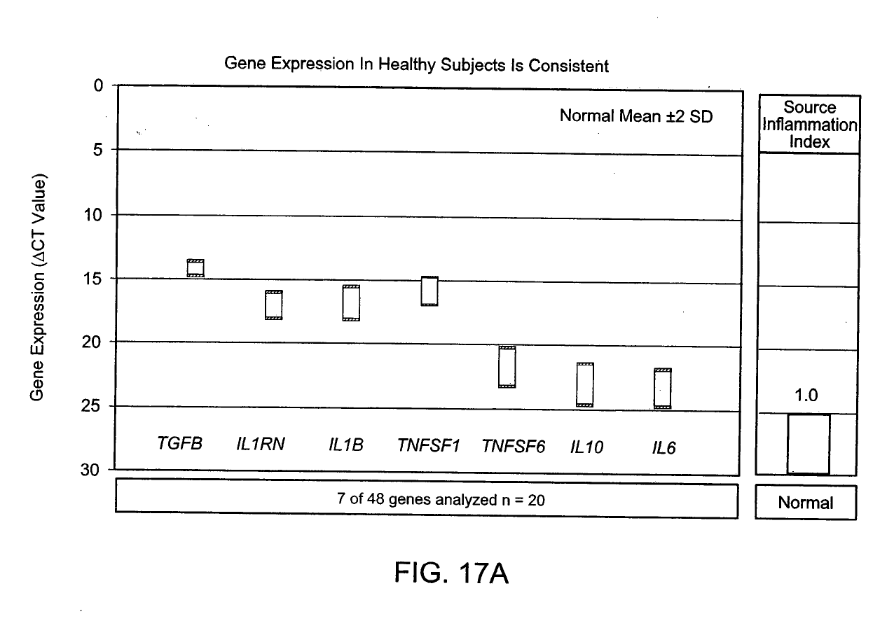

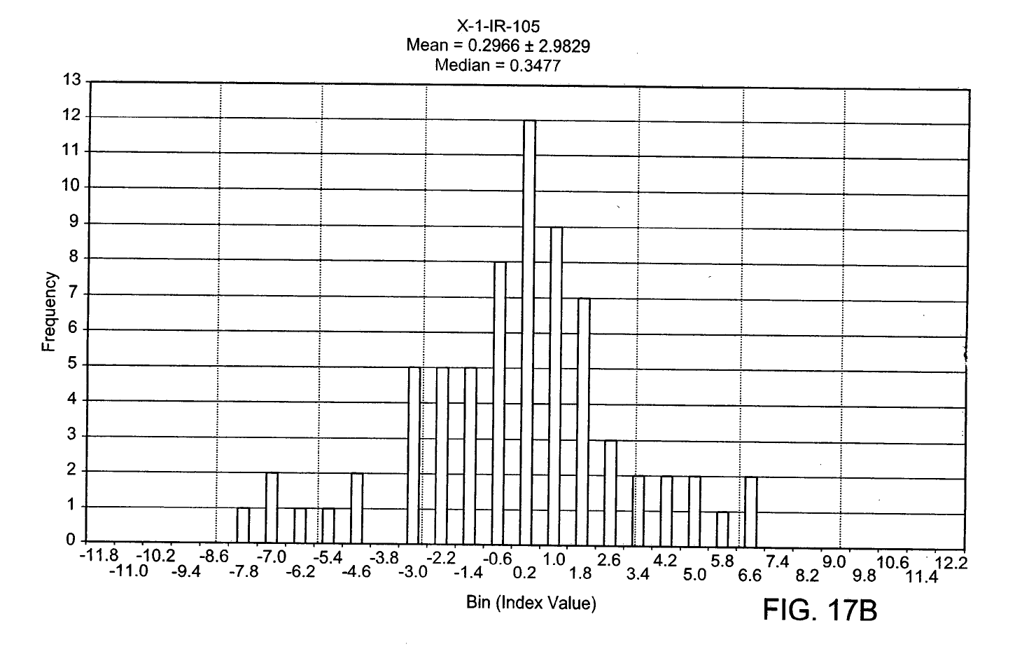

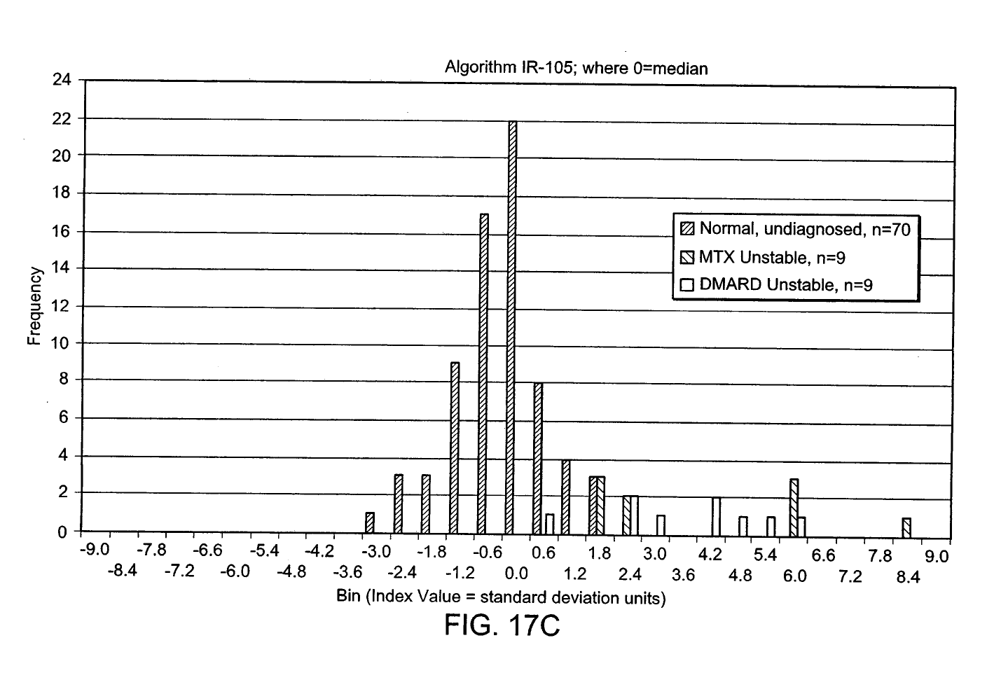

As an example, the index can be constructed, in relation to a normative Gene Expression Profile for a population or set of healthy subjects, in such a way that a reading of approximately 1 characterizes normative Gene Expression Profiles of healthy subjects. Let us further assume that the biological condition that is the subject of the index is inflammation; a reading of 1 in this example thus corresponds to a Gene Expression Profile that matches the norm for healthy subjects. A substantially higher reading then may identify a subject experiencing an inflammatory condition. The use of I as identifying a normative value, however, is only one possible choice; another logical choice is to use 0 as identifying the normative value. With this choice, deviations in the index from zero can be indicated in standard deviation units (so that values lying between −1 and +1 encompass 90% of a normally distributed reference population or set of subjects. Since we have found that Gene Expression Profile values (and accordingly constructed indices based on them) tend to be normally distributed, the 0-centered index constructed in this manner is highly informative. It therefore facilitates use of the index in diagnosis of disease and setting objectives for treatment. The choice of 0 for the normative value, and the use of standard deviation units, for example, are illustrated in FIG. 17B, discussed below.

### EXAMPLES

### Example 1

### Acute Inflammatory Index to Assist in Analysis of Large, Complex Data Sets

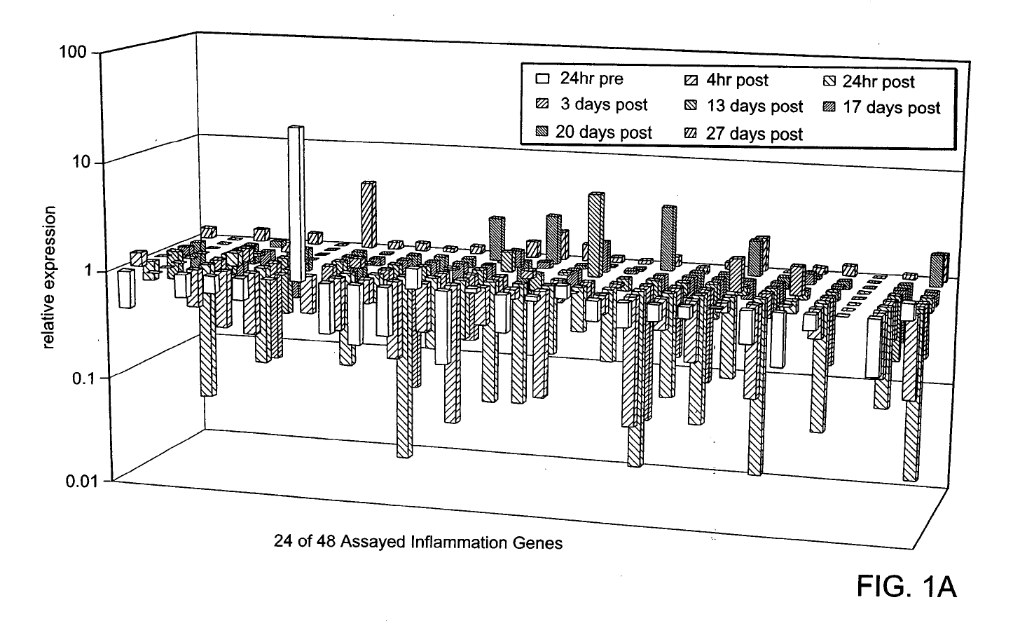

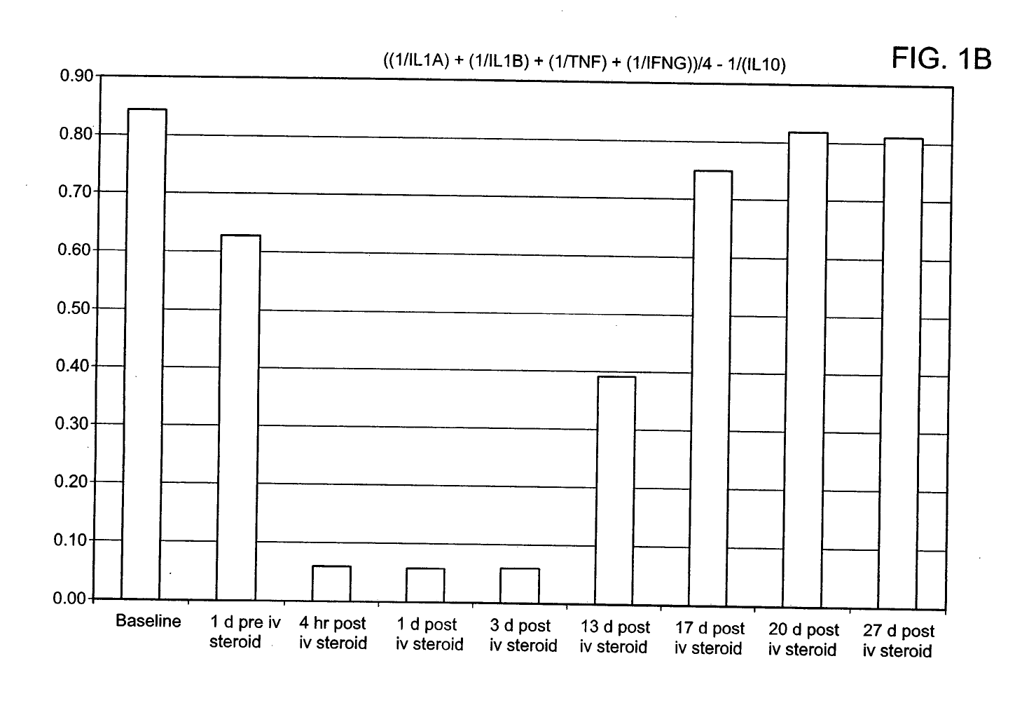

In one embodiment of the invention the index value or algorithm can be used to reduce a complex data set to a single index value that is informative with respect to the inflammatory state of a subject. This is illustrated in FIGS. 1A and 1B.

FIG. 1A is entitled Source Precision Inflammation Profile Tracking of A Subject Results in a Large, Complex Data Set. The figure shows the results of assaying 24 genes from the Inflammation Gene Expression Panel (shown in Table 1) on eight separate days during the course of optic neuritis in a single male subject.

FIG. 1B shows use of an Acute Inflammation Index. The data displayed in FIG. 1A above is shown in this figure after calculation using an index function proportional to the following mathematical expression: (¼{IL1A}+¼{IL1B}+¼{TNF}+¼{INFG}−1/{IL10}).

### Example 2

### Use of Acute Inflammation Index or Algorithm to Monitor a Biological Condition of a Sample or a Subject

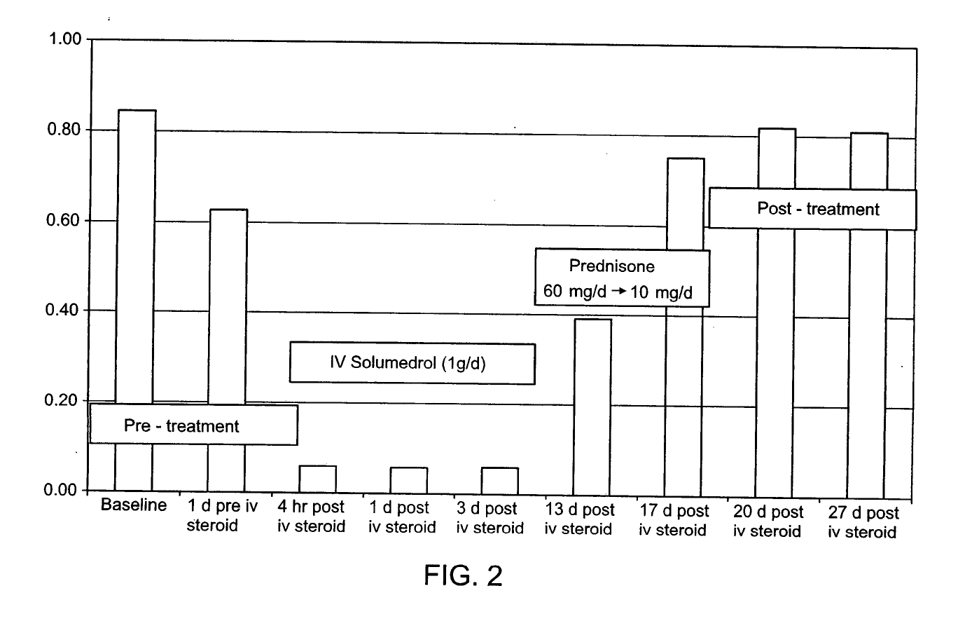

The inflammatory state of a subject reveals information about the past progress of the biological condition, future progress, response to treatment, etc. The Acute Inflammation Index may be used to reveal such information about the biological condition of a subject. This is illustrated in FIG. 2.

The results of the assay for inflammatory gene expression for each day (shown for 24 genes in each row of FIG. 1A) is displayed as an individual histogram after calculation. The index reveals clear trends in inflammatory status that may correlated with therapeutic intervention (FIG. 2).

FIG. 2 is a graphical illustration of the acute inflammation index calculated at 9 different, significant clinical milestones from blood obtained from a single patient treated medically with for optic neuritis. Changes in the index values for the Acute Inflammation Index correlate strongly with the expected effects of therapeutic intervention. Four clinical milestones have been identified on top of the Acute Inflammation Index in this figure including (1) prior to treatment with steroids, (2) treatment with IV solumedrol at 1 gram per day, (3) post-treatment with oral prednisone at 60 mg per day tapered to 10 mg per day and (4) post treatment. The data set is the same as for FIG. 1. The index is proportional to ¼{IL1A}+/4{IL1B}+¼{TNF}+¼{INFG}−1/{IL10}. As expected, the acute inflammation index falls rapidly with treatment with IV steroid, goes up during less efficacious treatment with oral prednisone and returns to the pre-treatment level after the steroids have been discontinued and metabolized completely.

### Example 3

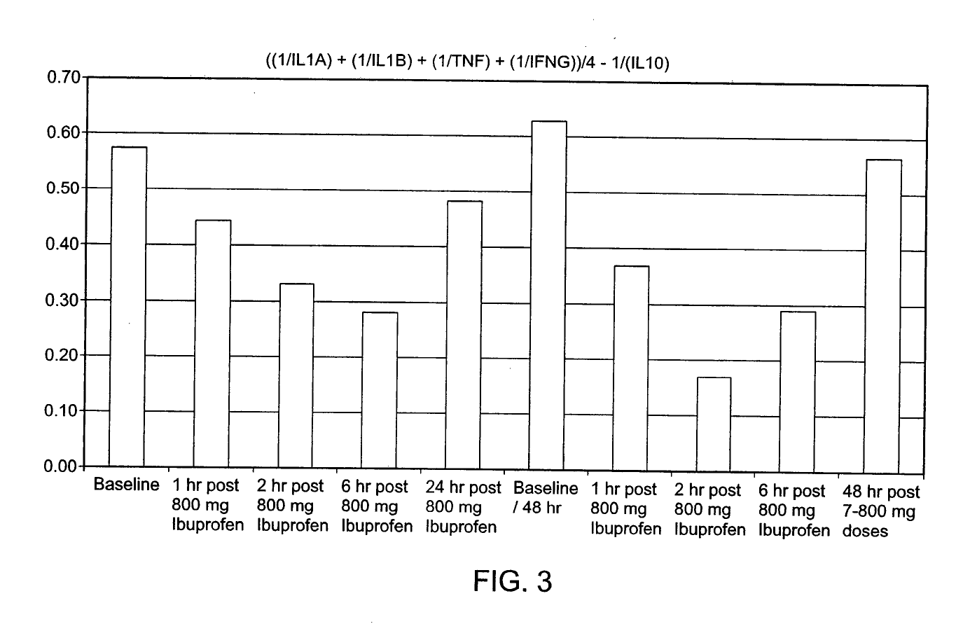

Use of the acute inflammatory index to set dose, including concentrations and timing, for compounds in development or for compounds to be tested in human and non-human subjects as shown in FIG. 3. The acute inflammation index may be used as a common reference value for therapeutic compounds or interventions without common mechanisms of action. The compound that induces a gene response to a compound as indicated by the index, but fails to ameliorate a known biological conditions may be compared to a different compounds with varying effectiveness in treating the biological condition.

FIG. 3 shows the effects of single dose treatment with 800 mg of ibuprofen in a single donor as characterized by the Acute Inflammation Index. 800 mg of over-the-counter ibuprofen were taken by a single subject at Time=0 and Time=48 hr. Gene expression values for the indicated five inflammation-related gene loci were determined as described below at times=2, 4, 6, 48, 50, 56 and 96 hours. As expected the acute inflammation index falls immediately after taking the non-steroidal anti-inflammatory ibuprofen and returns to baseline after 48 hours. A second dose at T=48 follows the same kinetics at the first dose and returns to baseline at the end of the experiment at T=96.

### Example 4

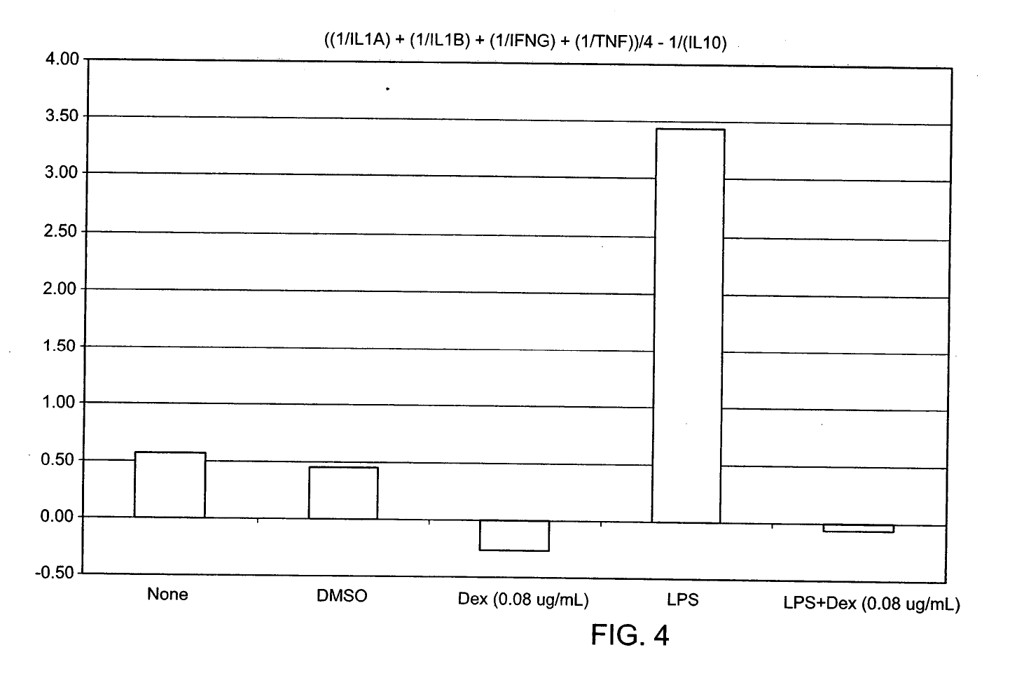

Use of the acute inflammation index to characterize efficacy, safety, and mode of physiological action for an agent, which may be in development and/or may be complex in nature. This is illustrated in FIG. 4.

FIG. 4 shows that the calculated acute inflammation index displayed graphically for five different conditions including (A) untreated whole blood; (B) whole blood treated in vitro with DMSO, an non-active carrier compound; (C) otherwise unstimulated whole blood treated in vitro with dexamethasone (0.08 ug/ml): (D) whole blood stimulated in vitro with lipopolysaccharide, a known pro-inflammatory compound, (LPS, 1 ng/ml) and (E) whole blood treated in vitro with LPS (1 ng/ml) and dexamethasone (0.08 ug/ml). Dexamethasone is used as a prescription compound that is commonly used medically as an anti-inflammatory steroid compound. The acute inflammation index is calculated from the experimentally determined gene expression levels of inflammation-related genes expressed in human whole blood obtained from a single patient. Results of mRNA expression are expressed as Ct's in this example, but may be expressed as, e.g., relative fluorescence units, copy number or any other quantifiable, precise and calibrated form, for the genes IL1A, IL1B, TNF, IFNG and IL10. From the gene expression values, the acute inflammation values were determined algebraically according in proportion to the expression ¼{IL1A}+¼{IL1B}+¼{TNF}+¼{INFG}−1/{IL10}.

### Example 5

### Development and Use of Population Normative Values for Gene Expression Profiles

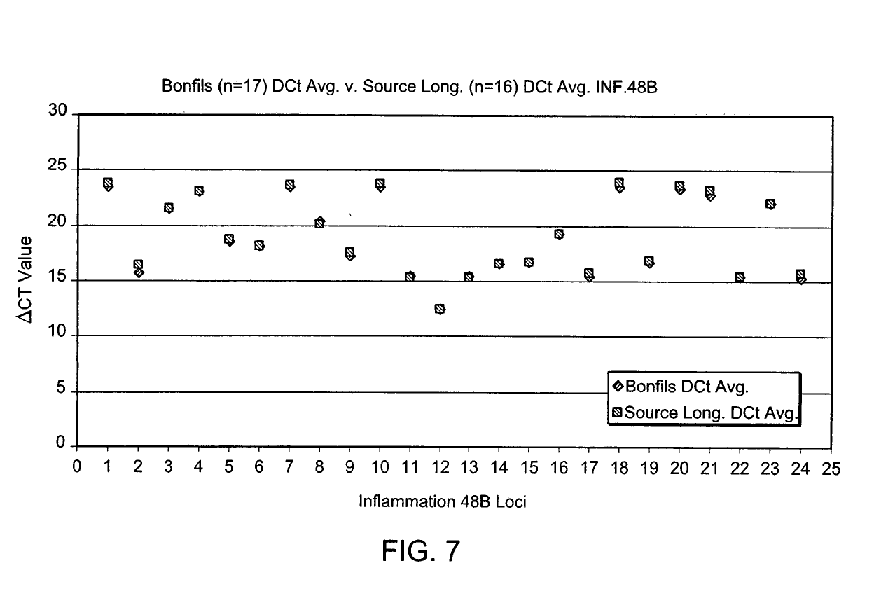

FIGS. 6 and 7 show the arithmetic mean values for gene expression profiles (using the 48 loci of the Inflammation Gene Expression Panel of Table 1) obtained from whole blood of two distinct patient populations (patient sets). These patient sets are both normal or undiagnosed. The first patient set, which is identified as Bonfils (the plot points for which are represented by diamonds), is composed of 17 subjects accepted as blood donors at the Bonfils Blood Center in Denver, Colo. The second patient set is 9 donors, for which Gene Expression Profiles were obtained from assays conducted four times over a four-week period. Subjects in this second patient set (plot points for which are represented by squares) were recruited from employees of Source Precision Medicine, Inc., the assignee herein. Gene expression averages for each population were calculated for each of 48 gene loci of the Gene Expression Inflammation Panel. The results for loci 1-24 (sometimes referred to below as the Inflammation 48A loci) are shown in FIG. 6 and for loci 25-48 (sometimes referred to below as the Inflammation 48B loci) are shown in FIG. 7.

The consistency between gene expression levels of the two distinct patient sets is dramatic. Both patient sets show gene expressions for each of the 48 loci that are not significantly different from each other. This observation suggests that there is a “normal” expression pattern for human inflammatory genes, that a Gene Expression Profile, using the Inflammation Gene Expression Panel of Table 1 (or a subset thereof) characterizes that expression pattern, and that a population-normal expression pattern can be used, for example, to guide medical intervention for any biological condition that results in a change from the normal expression pattern.

In a similar vein, FIG. 8 shows arithmetic mean values for gene expression profiles (again using the 48 loci of the Inflammation Gene Expression Panel of Table 1) also obtained from whole blood of two distinct patient populations (patient sets). One patient set, expression values for which are represented by triangular data points, is 24 normal, undiagnosed subjects (who therefore have no known inflammatory disease). The other patient set, the expression values for which are represented by diamond-shaped data points, is four patients with rheumatoid arthritis and who have failed therapy (who therefore have unstable rheumatoid arthritis).

As remarkable as the consistency of data from the two distinct normal patient sets shown in FIGS. 6 and 7 is the systematic divergence of data from the normal and diseased patient sets shown in FIG. 8. In 45 of the shown 48 inflammatory gene loci, subjects with unstable rheumatoid arthritis showed, on average, increased inflammatory gene expression (lower cycle threshold values; Ct), than subjects without disease. The data thus further demonstrate that is possible to identify groups with specific biological conditions using gene expression if the precision and calibration of the underlying assay are carefully designed and controlled according to the teachings herein.

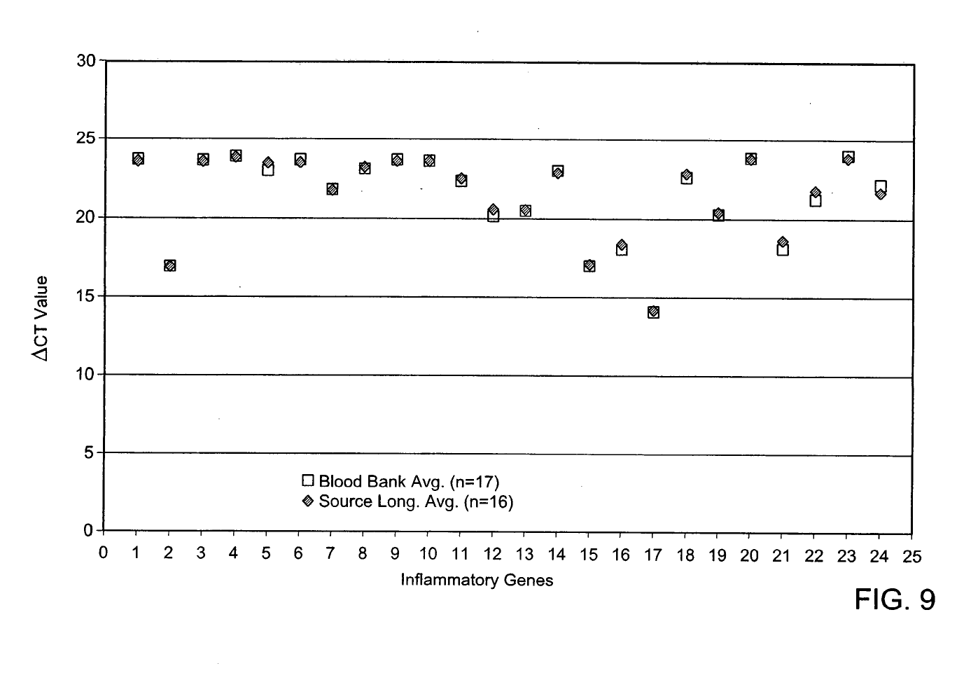

FIG. 9, in a manner analogous to FIG. 8, shows the shows arithmetic mean values for gene expression profiles using 24 loci of the Inflammation Gene Expression Panel of Table 1) also obtained from whole blood of two distinct patient sets. One patient set, expression values for which are represented by diamond-shaped data points, is 17 normal, undiagnosed subjects (who therefore have no known inflammatory disease) who are blood donors. The other patient set, the expression values for which are represented by square-shaped data points, is 16 subjects, also normal and undiagnosed, who have been monitored over six months, and the averages of these expression values are represented by the square-shaped data points. Thus the cross-sectional gene expression-value averages of a first healthy population match closely the longitudinal gene expression-value averages of a second healthy population, with approximately 7% or less variation in measured expression value on a gene-to-gene basis.

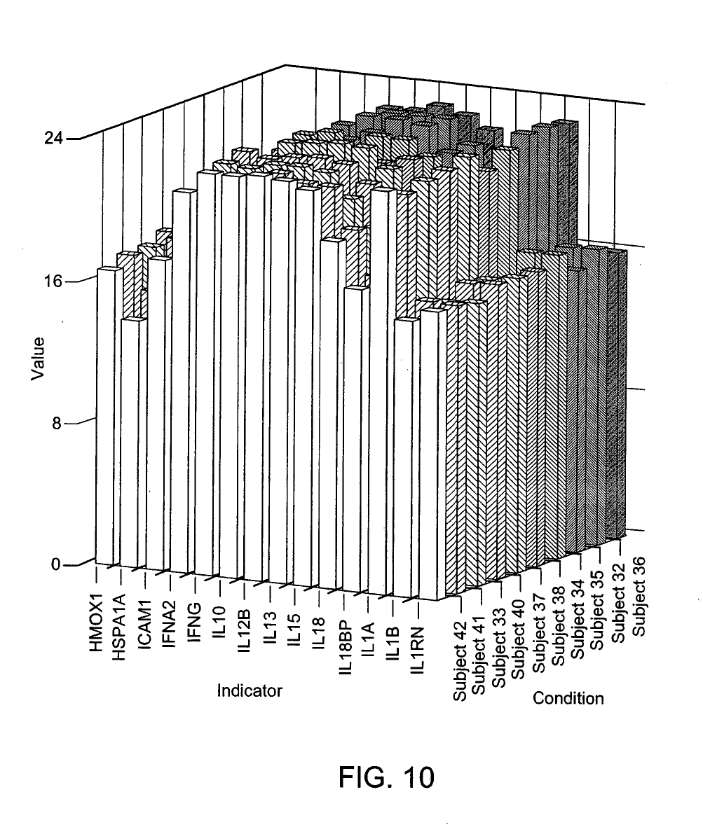

FIG. 10 shows the shows gene expression values (using 14 loci of the Inflammation Gene Expression Panel of Table 1) obtained from whole blood of 44 normal undiagnosed blood donors (data for 10 subjects of which is shown). Again, the gene expression values for each member of the population (set) are closely matched to those for the entire set, represented visually by the consistent peak heights for each of the gene loci. Other subjects of the set and other gene loci than those depicted here display results that are consistent with those shown here.

In consequence of these principles, and in various embodiments of the present invention, population normative values for a Gene Expression Profile can be used in comparative assessment of individual subjects as to biological condition, including both for purposes of health and/or disease. In one embodiment the normative values for a Gene Expression Profile may be used as a baseline in computing a “calibrated profile data set” (as defined at the beginning of this section) for a subject that reveals the deviation of such subject's gene expression from population normative values. Population normative values for a Gene Expression Profile can also be used as baseline values in constructing index functions in accordance with embodiments of the present invention. As a result, for example, an index function can be constructed to reveal not only the extent of an individual's inflammation expression generally but also in relation to normative values.

### Example 6

### Consistency of Expression Values, of Constituents in Gene Expression Panels, Over Time as Reliable Indicators of Biological Condition

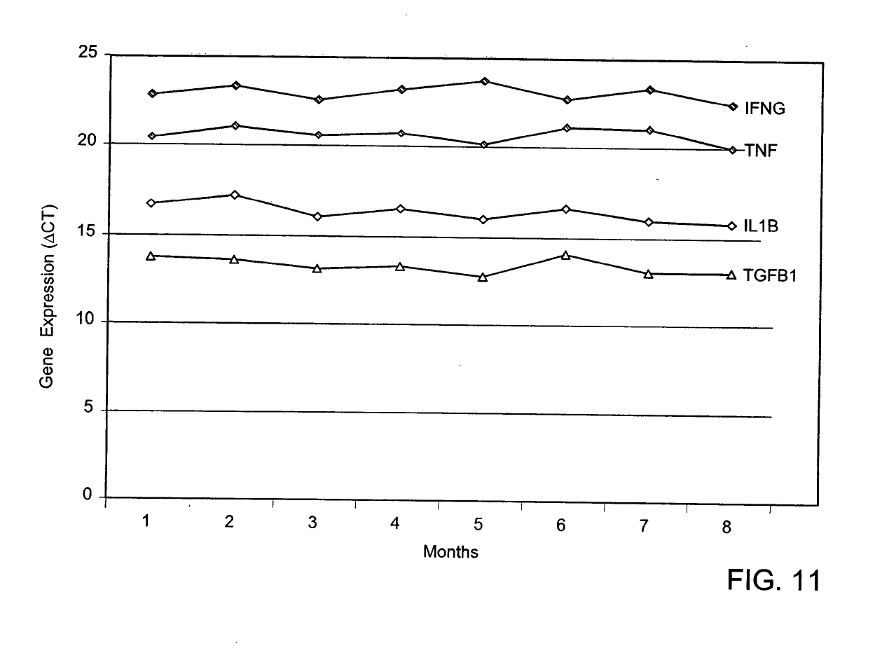

FIG. 11 shows the expression levels for each of four genes (of the Inflammation Gene Expression Panel of Table 1), of a single subject, assayed monthly over a period of eight months. It can be seen that the expression levels are remarkably consistent over time.

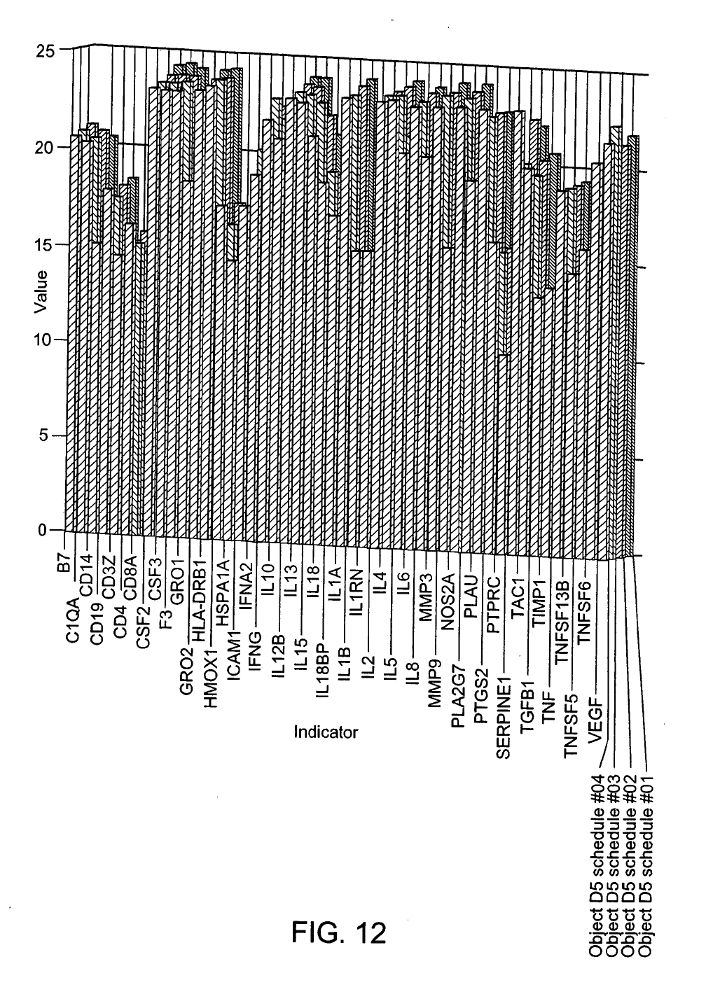

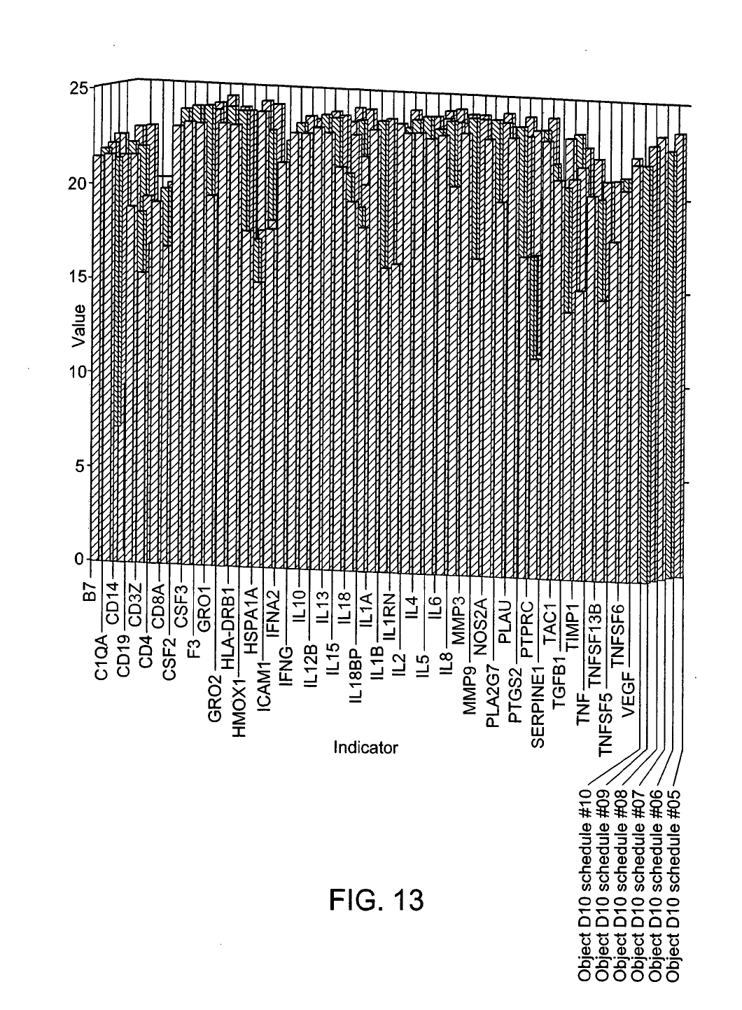

FIGS. 12 and 13 similarly show in each case the expression levels for each of 48 genes (of the Inflammation Gene Expression Panel of Table 1), of distinct single subjects (selected in each case on the basis of feeling well and not taking drugs), assayed, in the case of FIG. 12 weekly over a period of four weeks, and in the case of FIG. 13 monthly over a period of six months. In each case, again the expression levels are remarkably consistent over time, and also similar across individuals.

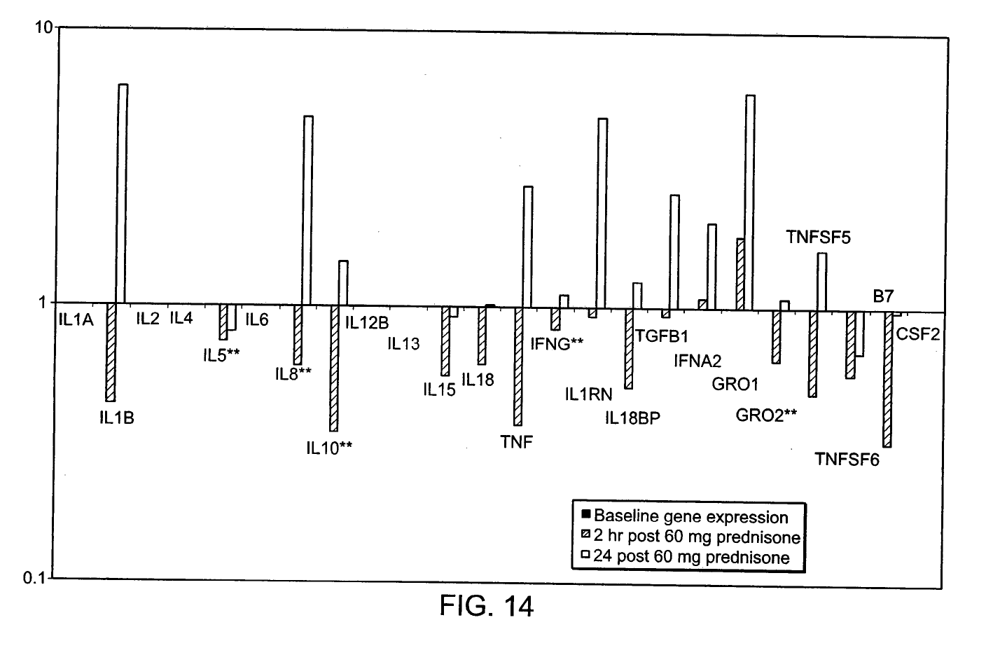

FIG. 14 also shows the effect over time, on inflammatory gene expression in a single human subject, of the administration of an anti-inflammatory steroid, as assayed using the Inflammation Gene Expression Panel of Table 1. In this case, 24 of 48 loci are displayed. The subject had a baseline blood sample drawn in a PAX RNA isolation tube and then took a single 60 mg dose of prednisone, an anti-inflammatory, prescription steroid. Additional blood samples were drawn at 2 hr and 24 hr post the single oral dose. Results for gene expression are displayed for all three time points, wherein values for the baseline sample are shown as unity on the x-axis. As expected, oral treatment with prednisone resulted in the decreased expression of most of inflammation-related gene loci, as shown by the 2-hour post-administration bar graphs. However, the 24-hour post-administration bar graphs show that, for most of the gene loci having reduced gene expression at 2 hours, there were elevated gene expression levels at 24 hr.

Although the baseline in FIG. 14 is based on the gene expression values before drug intervention associated with the single individual tested, we know from the previous example, that healthy individuals tend toward population normative values in a Gene Expression Profile using the Inflammation Gene Expression Panel of Table 1 (or a subset of it). We conclude from FIG. 14 that in an attempt to return the inflammatory gene expression levels to those demonstrated in FIGS. 6 and 7 (normal or set levels), interference with the normal expression induced a compensatory gene expression response that over-compensated for the drug-induced response, perhaps because the prednisone had been significantly metabolized to inactive forms or eliminated from the subject.

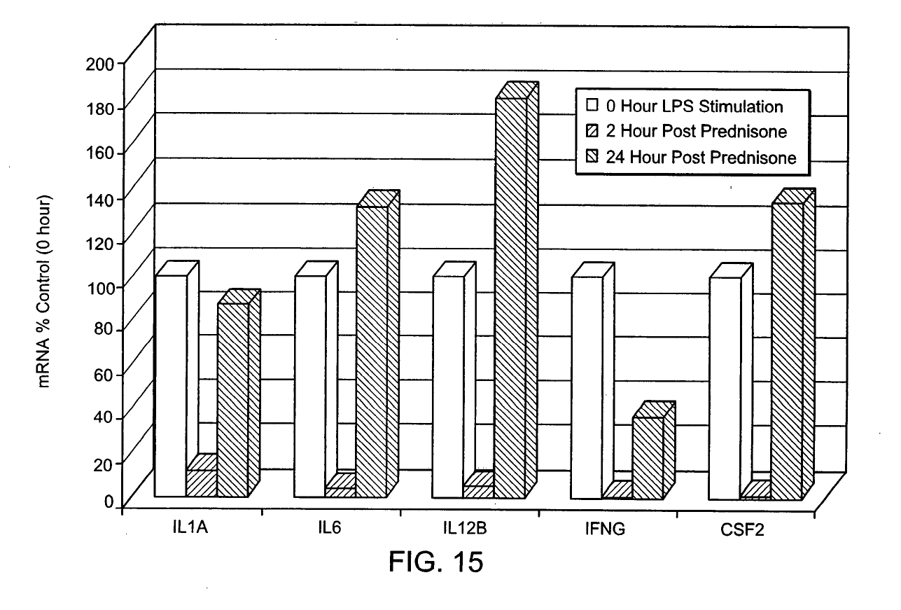

FIG. 15, in a manner analogous to FIG. 14, shows the effect over time, via whole blood samples obtained from a human subject, administered a single dose of prednisone, on expression of 5 genes (of the Inflammation Gene Expression Panel of Table 1). The samples were taken at the time of administration (t=0) of the prednisone, then at two and 24 hours after such administration. Each whole blood sample was challenged by the addition of 0.1 ng/ml of lipopolysaccharide (a Gram-negative endotoxin) and a gene expression profile of the sample, post-challenge, was determined. It can seen that the two-hour sample shows dramatically reduced gene expression of the 5 loci of the Inflammation Gene Expression Panel, in relation to the expression levels at the time of administration (t=0). At 24 hours post administration, the inhibitory effect of the prednisone is no longer apparent, and at 3 of the 5 loci, gene expression is in fact higher than at t=0, illustrating quantitatively at the molecular level the well-known rebound effect.

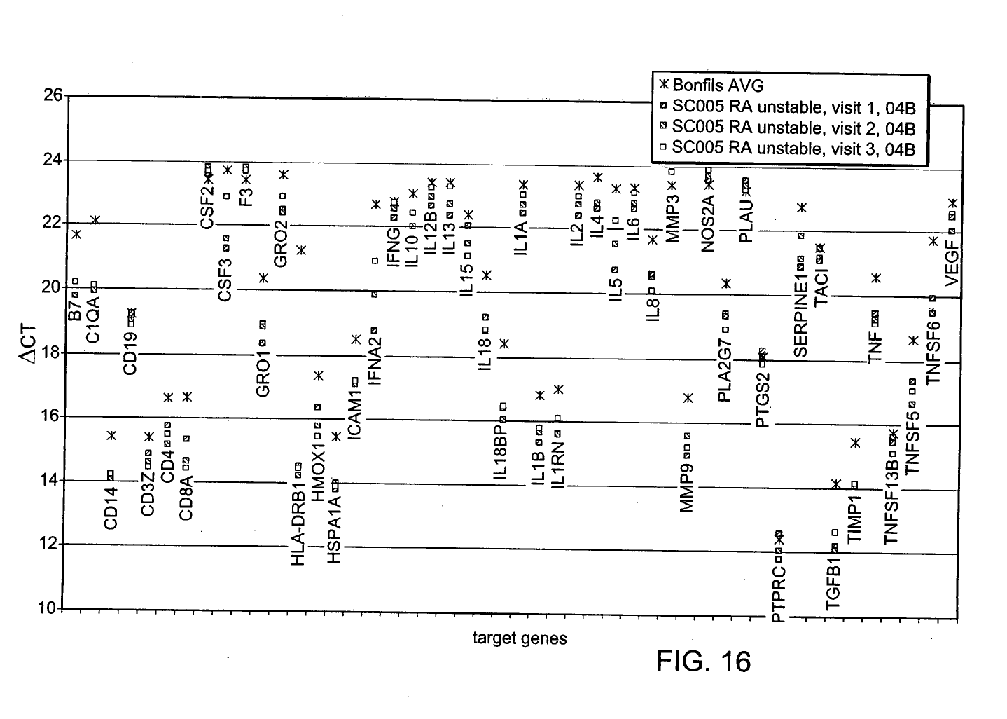

FIG. 16 also shows the effect over time, on inflammatory gene expression in a single human subject suffering from rheumatoid arthritis, of the administration of a TNF-inhibiting compound, but here the expression is shown in comparison to the cognate locus average previously determined (in connection with FIGS. 6 and 7) for the normal (i.e., undiagnosed, healthy) patient set. As part of a larger international study involving patients with rheumatoid arthritis, the subject was followed over a twelve-week period. The subject was enrolled in the study because of a failure to respond to conservative drug therapy for rheumatoid arthritis and a plan to change therapy and begin immediate treatment with a TNF-inhibiting compound. Blood was drawn from the subject prior to initiation of new therapy (visit 1). After initiation of new therapy, blood was drawn at 4 weeks post change in therapy (visit 2), 8 weeks (visit 3), and 12 weeks (visit 4) following the start of new therapy. Blood was collected in PAX RNA isolation tubes, held at room temperature for two hours and then frozen at −30° C.

Frozen samples were shipped to the central laboratory at Source Precision Medicine, the assignee herein, in Boulder, Colo. for determination of expression levels of genes in the 48-gene Inflammation Gene Expression Panel of Table 1. The blood samples were thawed and RNA extracted according to the manufacturer's recommended procedure. RNA was converted to cDNA and the level of expression of the 48 inflammatory genes was determined. Expression results are shown for 11 of the 48 loci in FIG. 16. When the expression results for the 11 loci are compared from visit one to a population average of normal blood donors from the United States, the subject shows considerable difference. Similarly, gene expression levels at each of the subsequent physician visits for each locus are compared to the same normal average value. Data from visits 2, 3 and 4 document the effect of the change in therapy. In each visit following the change in the therapy, the level of inflammatory gene expression for 10 of the 11 loci is closer to the cognate locus average previously determined for the normal (i.e., undiagnosed, healthy) patient set.

FIG. 17A further illustrates the consistency of inflammatory gene expression, illustrated here with respect to 7 loci of (of the Inflammation Gene Expression Panel of Table 1), in a set of 44 normal, undiagnosed blood donors. For each individual locus is shown the range of values lying within ±2 standard deviations of the mean expression value, which corresponds to 95% of a normally distributed population. Notwithstanding the great width of the confidence interval (95%), the measured gene expression value (ΔCT)—remarkably—still lies within 10% of the mean, regardless of the expression level involved. As described in further detail below, for a given biological condition an index can be constructed to provide a measurement of the condition. This is possible as a result of the conjunction of two circumstances: (i) there is a remarkable consistency of Gene Expression Profiles with respect to a biological condition across a population and (ii) there can be employed procedures that provide substantially reproducible measurement of constituents in a Gene Expression Panel giving rise to a Gene Expression Profile, under measurement conditions wherein specificity and efficiencies of amplification for all constituents of the panel are substantially similar and which therefore provides a measurement of a biological condition. Accordingly, a function of the expression values of representative constituent loci of FIG. 17A is here used to generate an inflammation index value, which is normalized so that a reading of 1 corresponds to constituent expression values of healthy subjects, as shown in the right-hand portion of FIG. 17A.

In FIG. 17B, an inflammation index value was determined for each member of a set of 42 normal undiagnosed blood donors, and the resulting distribution of index values, shown in the figure, can be seen to approximate closely a normal distribution, notwithstanding the relatively small subject set size. The values of the index are shown relative to a 0-based median, with deviations from the median calibrated in standard deviation units. Thus 90% of the subject set lies within +1 and −1 of a 0 value. We have constructed various indices, which exhibit similar behavior.

FIG. 17C illustrates the use of the same index as FIG. 17B, where the inflammation median for a normal population of subjects has been set to zero and both normal and diseased subjects are plotted in standard deviation units relative to that median. An inflammation index value was determined for each member of a normal, undiagnosed population of 70 individuals (black bars). The resulting distribution of index values, shown in FIG. 17C, can be seen to approximate closely a normal distribution. Similarly, index values were calculated for individuals from two diseased population groups, (1) rheumatoid arthritis patients treated with methotrexate (MTX) who are about to change therapy to more efficacious drugs (e.g., TNF inhibitors)(hatched bars), and (2) rheumatoid arthritis patients treated with disease modifying anti-rheumatoid drugs (DMARDS) other than MTX, who are about to change therapy to more efficacious drugs (e.g., MTX). Both populations of subjects present index values that are skewed upward (demonstrating increased inflammation) in comparison to the normal distribution. This figure thus illustrates the utility of an index to derived from Gene Expression Profile data to evaluate disease status and to provide an objective and quantifiable treatment objective. When these two populations of subjects were treated appropriately, index values from both populations returned to a more normal distribution (data not shown here).

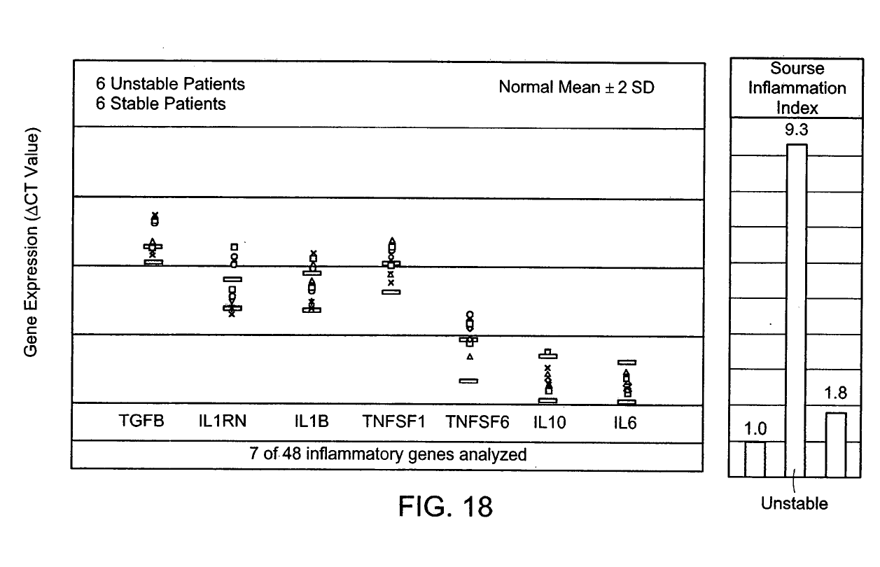

FIG. 18 plots, in a fashion similar to that of FIG. 17A, Gene Expression Profiles, for the same 7 loci as in FIG. 17A, two different 6-subject populations of rheumatoid arthritis patients. One population (called “stable” in the figure) is of patients who have responded well to treatment and the other population (called “unstable” in the figure) is of patients who have not responded well to treatment and whose therapy is scheduled for change. It can be seen that the expression values for the stable patient population, lie within the range of the 95% confidence interval, whereas the expression values for the unstable patient population for 5 of the 7 loci are outside and above this range. The right-hand portion of the figure shows an average inflammation index of 9.3 for the unstable population and an average inflammation index of 1.8 for the stable population, compared to I for a normal undiagnosed population of patients. The index thus provides a measure of the extent of the underlying inflammatory condition, in this case, rheumatoid arthritis. Hence the index, besides providing a measure of biological condition, can be used to measure the effectiveness of therapy as well as to provide a target for therapeutic intervention.

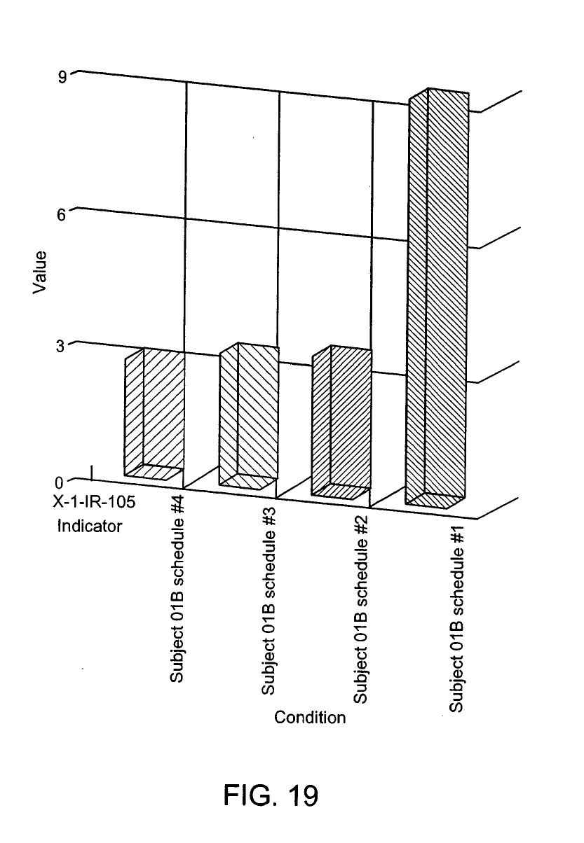

FIG. 19 thus illustrates use of the inflammation index for assessment of a single subject suffering from rheumatoid arthritis, who has not responded well to traditional therapy with methotrexate. The inflammation index for this subject is shown on the far right at start of a new therapy (a TNF inhibitor), and then, moving leftward, successively, 2 weeks, 6 weeks, and 12 weeks thereafter. The index can be seen moving towards normal, consistent with physician observation of the patient as responding to the new treatment.

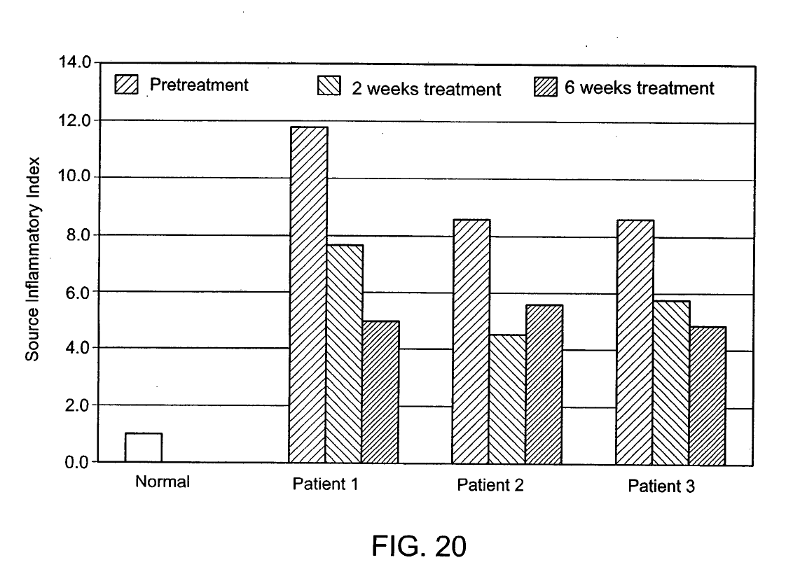

FIG. 20 similarly illustrates use of the inflammation index for assessment of three subjects suffering from rheumatoid arthritis, who have not responded well to traditional therapy with methotrexate, at the beginning of new treatment (also with a TNF inhibitor), and 2 weeks and 6 weeks thereafter. The index in each case can again be seen moving generally towards normal, consistent with physician observation of the patients as responding to the new treatment.

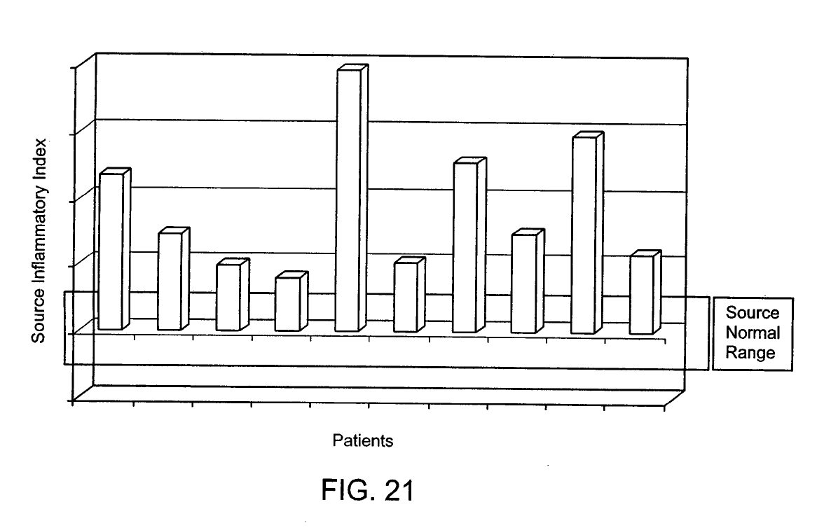

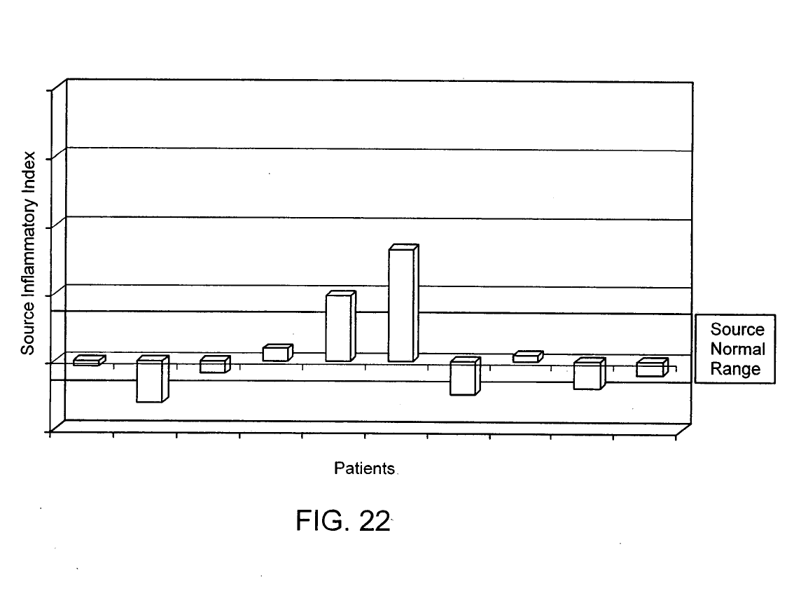

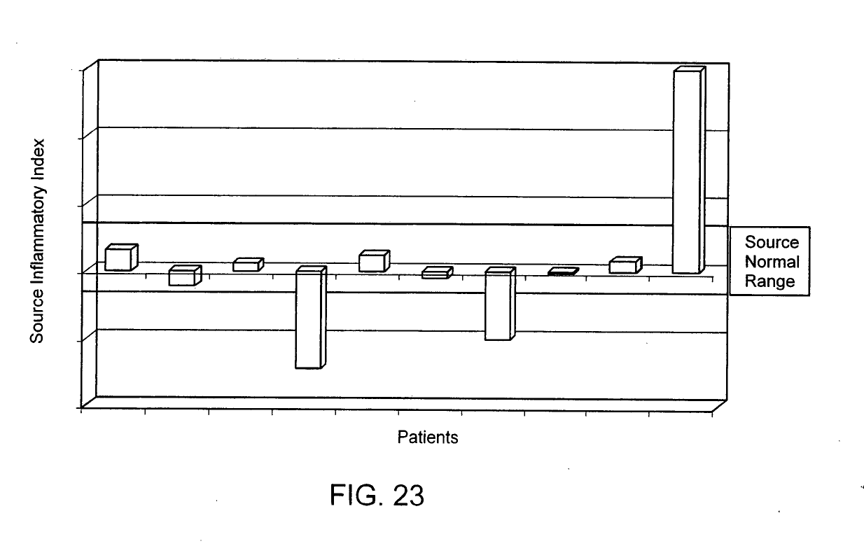

Each of FIGS. 21-23 shows the inflammation index for an international group of subjects, suffering from rheumatoid arthritis, each of whom has been characterized as stable (that is, not anticipated to be subjected to a change in therapy) by the subject's treating physician. FIG. 21 shows the index for each of 10 patients in the group being treated with methotrexate, which known to alleviate symptoms without addressing the underlying disease. FIG. 22 shows the index for each of 10 patients in the group being treated with Enbrel (an TNF inhibitor), and FIG. 23 shows the index for each 10 patients being treated with Remicade (another TNF inhibitor). It can be seen that the inflammation index for each of the patients in FIG. 21 is elevated compared to normal, whereas in FIG. 22, the patients being treated with Enbrel as a class have an inflammation index that comes much closer to normal (80% in the normal range). In FIG. 23, it can be seen that, while all but one of the patients being treated with Remicade have an inflammation index at or below normal, two of the patients have an abnormally low inflammation index, suggesting an immunosuppressive response to this drug. (Indeed, studies have shown that Remicade has been associated with serious infections in some subjects, and here the immunosuppressive effect is quantified.) Also in FIG. 23, one subject has an inflammation index that is significantly above the normal range. This subject in fact was also on a regimen of an anti-inflammation steroid (prednisone) that was being tapered; within approximately one week after the inflammation index was sampled, the subject experienced a significant flare of clinical symptoms.

Remarkably, these examples show a measurement, derived from the assay of blood taken from a subject, pertinent to the subject's arthritic condition. Given that the measurement pertains to the extent of inflammation, it can be expected that other inflammation-based conditions, including, for example, cardiovascular disease, may be monitored in a similar fashion.

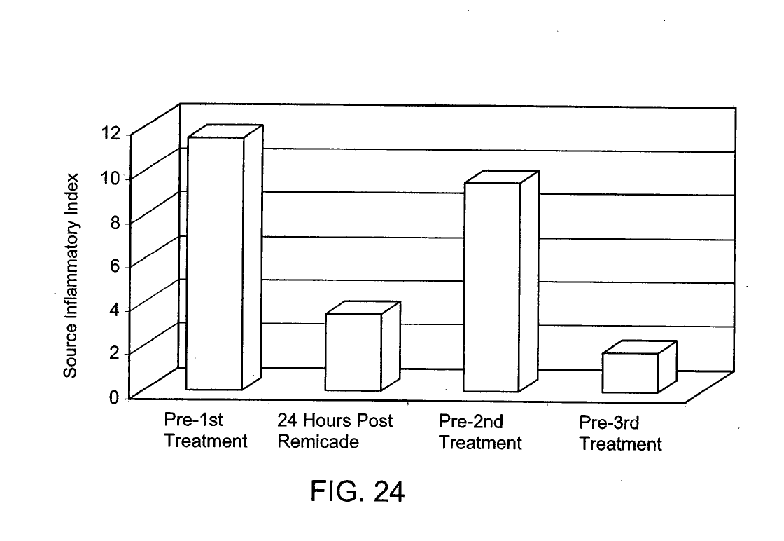

FIG. 24 illustrates use of the inflammation index for assessment of a single subject suffering from inflammatory bowel disease, for whom treatment with Remicade was initiated in three doses. The graphs show the inflammation index just prior to first treatment, and then 24 hours after the first treatment; the index has returned to the normal range. The index was elevated just prior to the second dose, but in the normal range prior to the third dose. Again, the index, besides providing a measure of biological condition, is here used to measure the effectiveness of therapy (Remicade), as well as to provide a target for therapeutic intervention in terms of both dose and schedule.

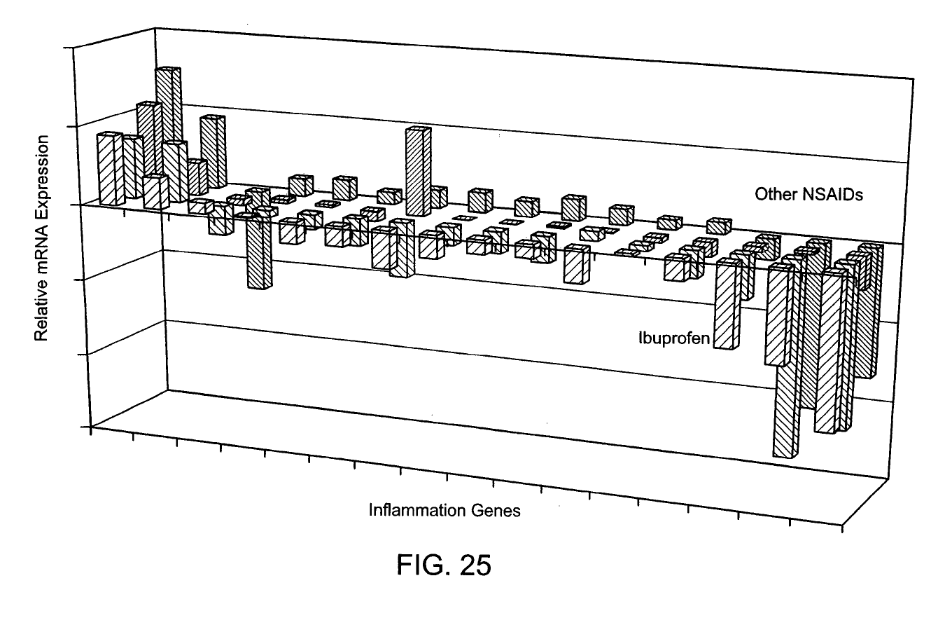

FIG. 25 shows Gene Expression Profiles with respect to 24 loci (of the Inflammation Gene Expression Panel of Table 1) for whole blood treated with Ibuprofen in vitro in relation to other non-steroidal anti-inflammatory drugs (NSAIDs). The profile for Ibuprofen is in front. It can be seen that all of the NSAIDs, including Ibuprofen share a substantially similar profile, in that the patterns of gene expression across the loci are similar. Notwithstanding these similarities, each individual drug has its own distinctive signature.

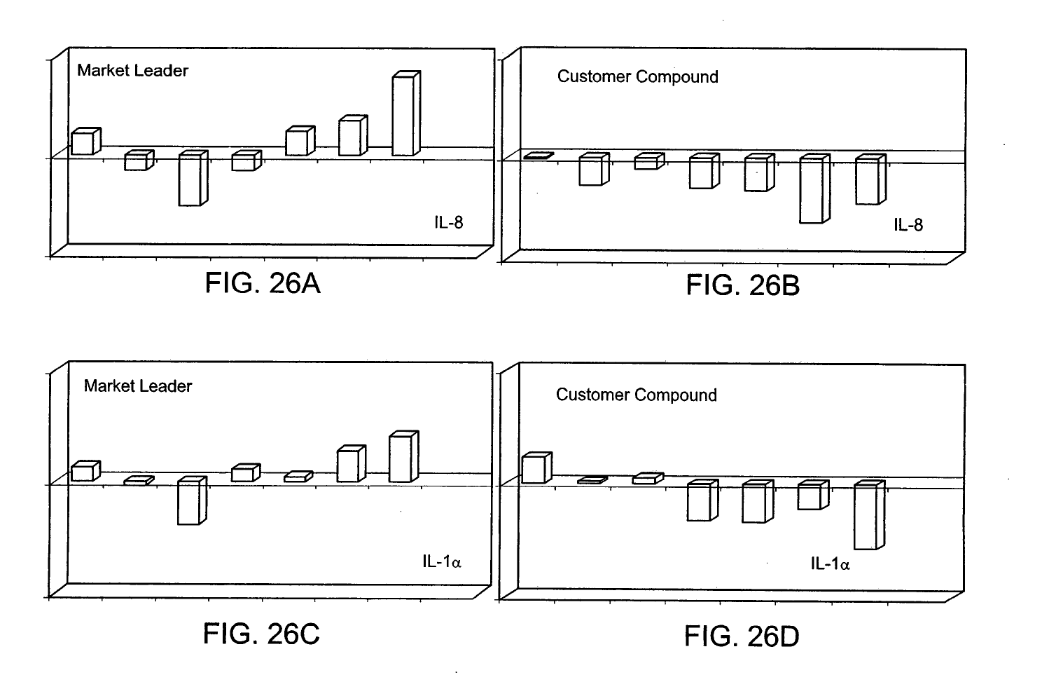

FIG. 26 illustrates how the effects of two competing anti-inflammatory compounds can be compared objectively, quantitatively, precisely, and reproducibly. In this example, expression of each of a panel of two genes (of the Inflammation Gene Expression Panel of Table 1) is measured for varying doses (0.08-250 μg/ml) of each drug in vitro in whole blood. The market leader drug shows a complex relationship between dose and inflammatory gene response. Paradoxically, as the dose is increased, gene expression for both loci initially drops and then increases in the case the case of the market leader. For the other compound, a more consistent response results, so that as the dose is increased, the gene expression for both loci decreases more consistently.

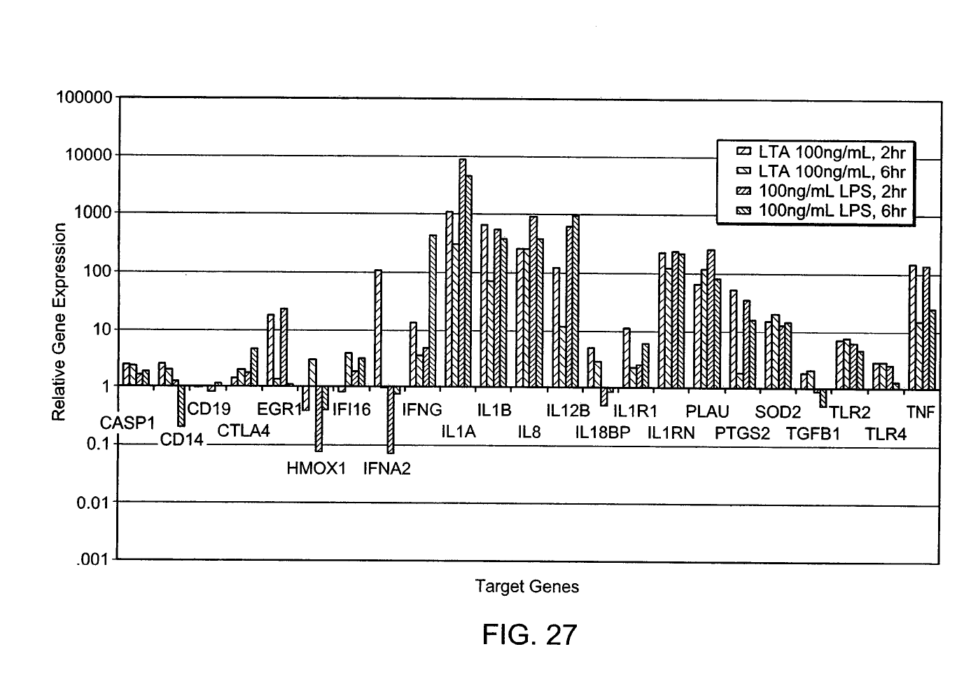

FIGS. 27 through 41 illustrate the use of gene expression panels in early identification and monitoring of infectious disease. These figures plot the response, in expression products of the genes indicated, in whole blood, to the administration of various infectious agents or products associated with infectious agents. In each figure, the gene expression levels are “calibrated”, as that term is defined herein, in relation to baseline expression levels determined with respect to the whole blood prior to administration of the relevant infectious agent. In this respect the figures are similar in nature to various figures of our below-referenced patent application WO 01/25473 (for example, FIG. 15 therein). The concentration change is shown ratiometrically, and the baseline level of 1 for a particular gene locus corresponds to an expression level for such locus that is the same, monitored at the relevant time after addition of the infectious agent or other stimulus, as the expression level before addition of the stimulus. Ratiometric changes in concentration are plotted on a logarithmic scale. Bars below the unity line represent decreases in concentration and bars above the unity line represent increases in concentration, the magnitude of each bar indicating the magnitude of the ratio of the change. We have shown in WO 01/25473 and other experiments that, under appropriate conditions, Gene Expression Profiles derived in vitro by exposing whole blood to a stimulus can be representative of Gene Expression Profiles derived in vivo with exposure to a corresponding stimulus.

FIG. 27 uses a novel bacterial Gene Expression Panel of 24 genes, developed to discriminate various bacterial conditions in a host biological system. Two different stimuli are employed: lipotechoic acid (LTA), a gram positive cell wall constituent, and lipopolysaccharide (LPS), a gram negative cell wall constituent. The final concentration immediately after administration of the stimulus was 100 ng/mL, and the ratiometric changes in expression, in relation to pre-administration levels, were monitored for each stimulus 2 and 6 hours after administration. It can be seen that differential expression can be observed as early as two hours after administration, for example, in the IFNA2 locus, as well as others, permitting discrimination in response between gram positive and gram negative bacteria.

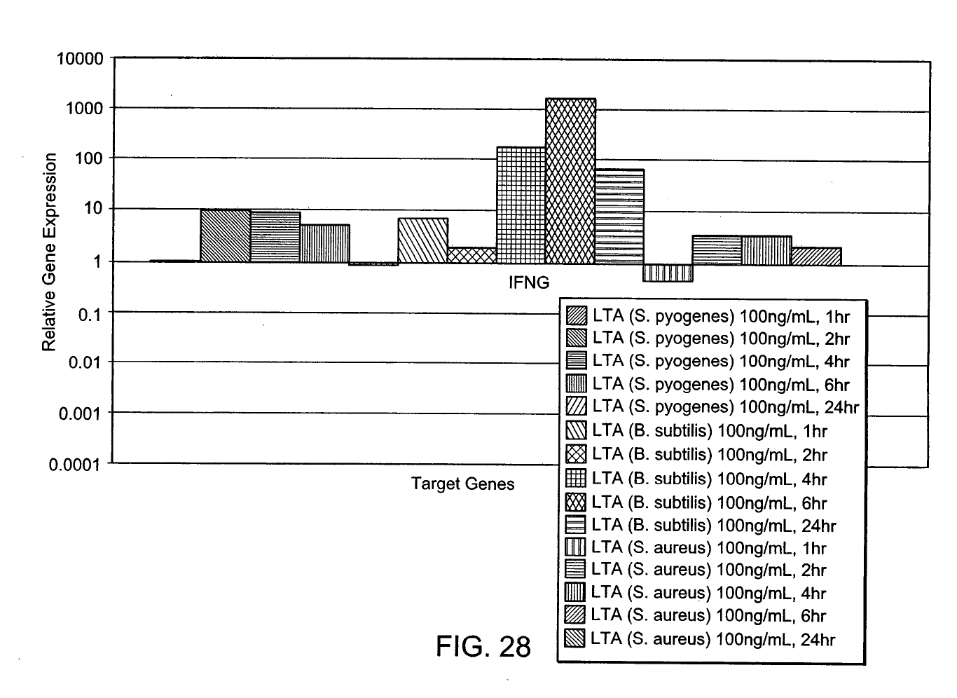

FIG. 28 shows differential expression for a single locus, IFNG, to LTA derived from three distinct sources: S. pyogenes, B. subtilis, and S. aureus. Each stimulus was administered to achieve a concentration of 100 ng/mL, and the response was monitored at 1, 2, 4, 6, and 24 hours after administration. The results suggest that Gene Expression Profiles can be used to distinguish among different infectious agents, here different species of gram positive bacteria.

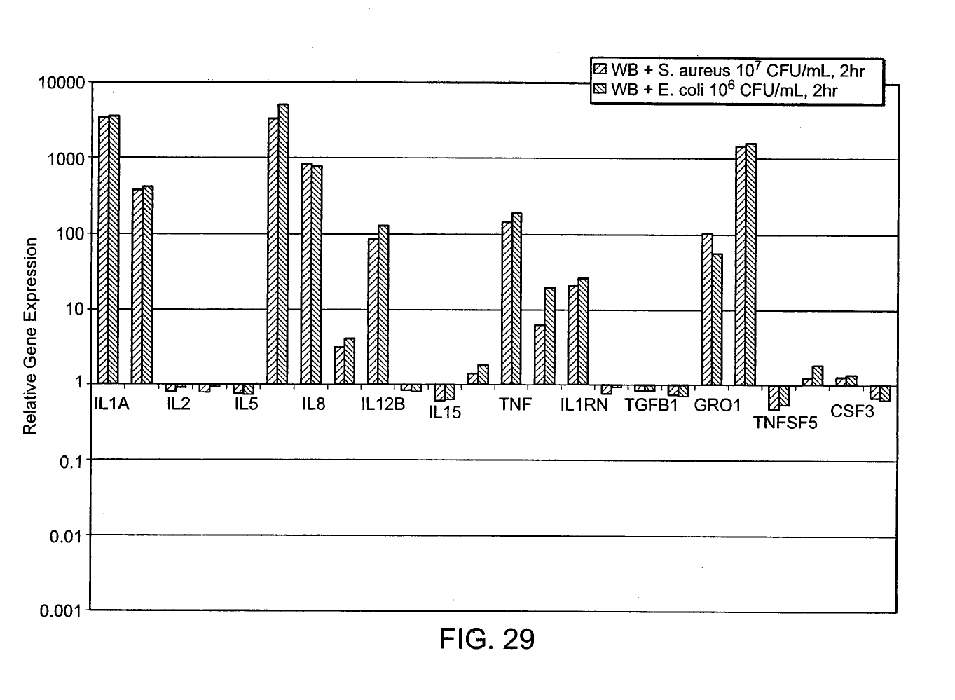

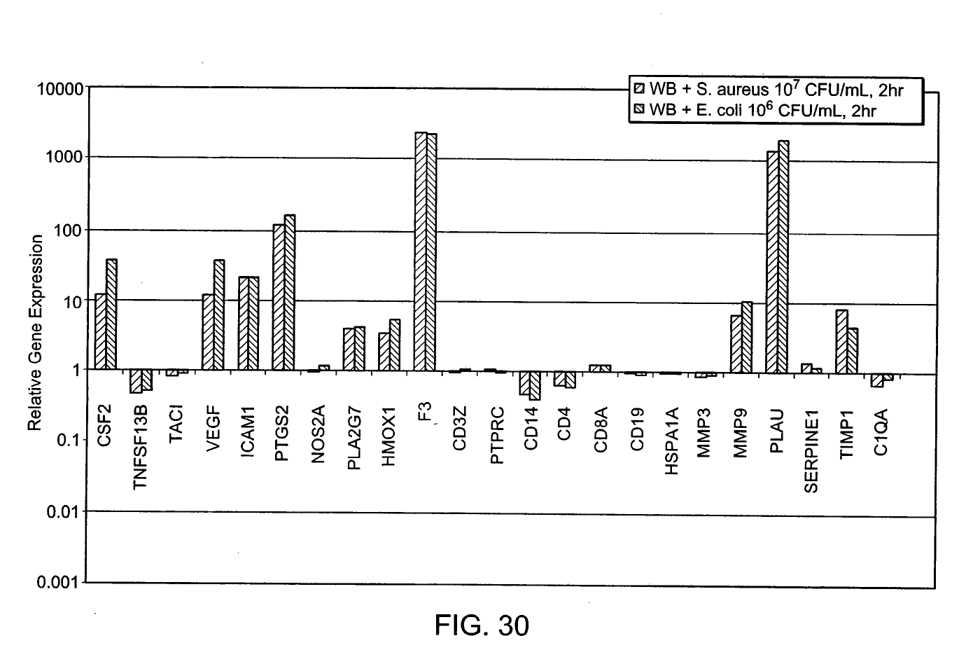

FIGS. 29 and 30 show the response of the Inflammation 48A and 48B loci respectively (discussed above in connection with FIGS. 6 and 7 respectively) in whole blood to administration of a stimulus of S. aureus and of a stimulus of E. coli (in the indicated concentrations, just after administration, of 10⁷ and 10⁶ CFU/mL respectively), monitored 2 hours after administration in relation to the pre-administration baseline. The figures show that many of the loci respond to the presence of the bacterial infection within two hours after infection.

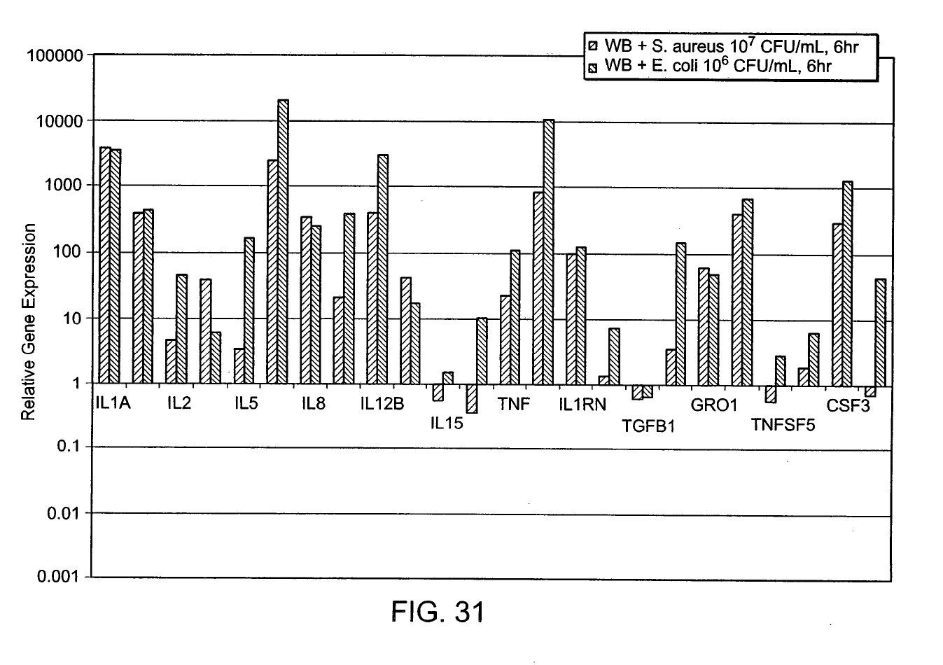

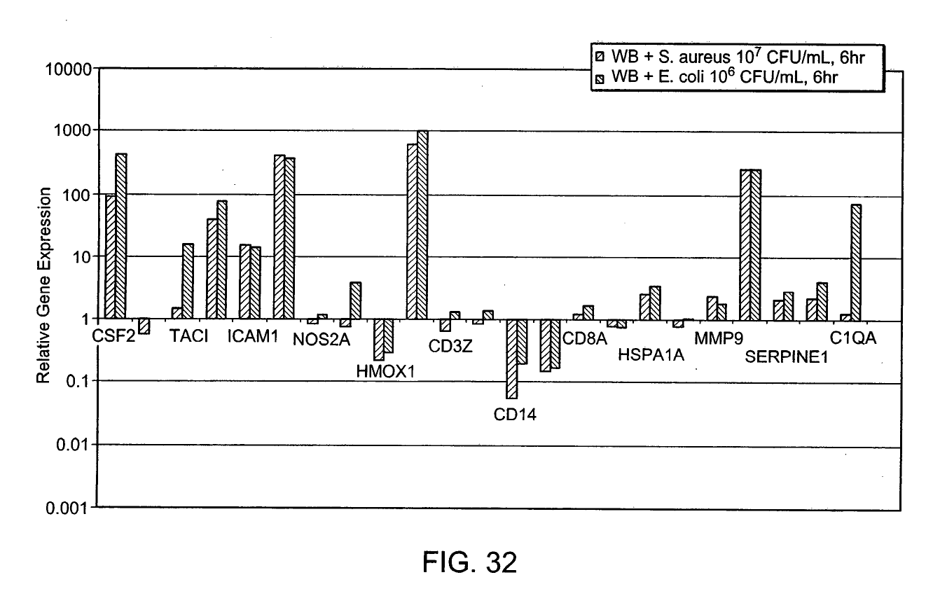

FIGS. 31 and 32 correspond to FIGS. 29 and 30 respectively and are similar to them, with the exception that the monitoring here occurs 6 hours after administration. More of the loci are responsive to the presence of infection. Various loci, such as IL2, show expression levels that discriminate between the two infectious agents.

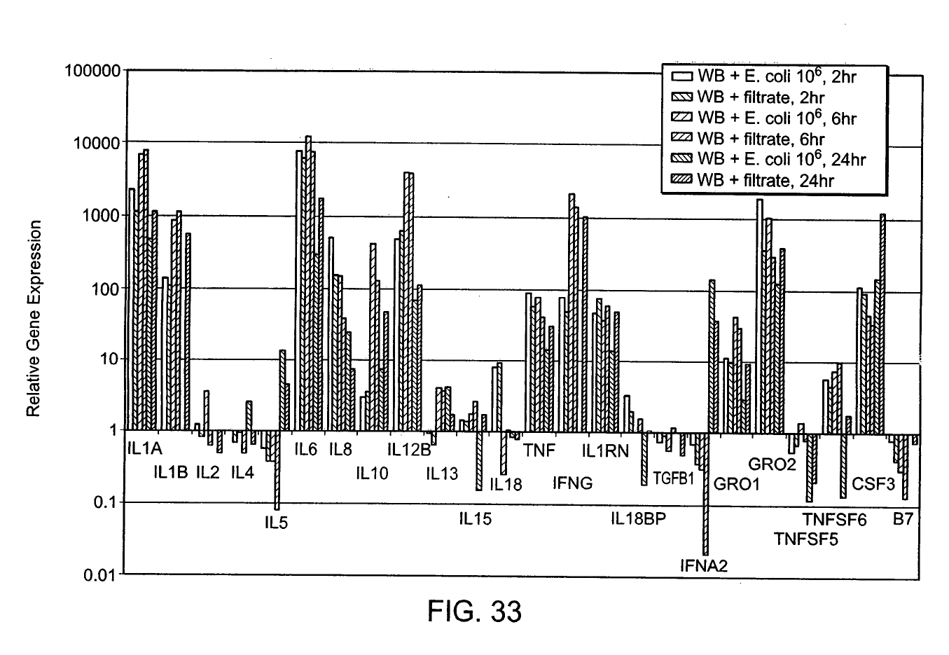

FIG. 33 shows the response of the Inflammation 48A loci to the administration of a stimulus of E. coli (again in the concentration just after administration of 10⁶ CFU/mL) and to the administration of a stimulus of an E. coli filtrate containing E. coli bacteria by products but lacking E. coli bacteria. The responses were monitored at 2, 6, and 24 hours after administration. It can be seen, for example, that the responses over time of loci IL1B, IL18 and CSF3 to E. coli and to E. coli filtrate are different.

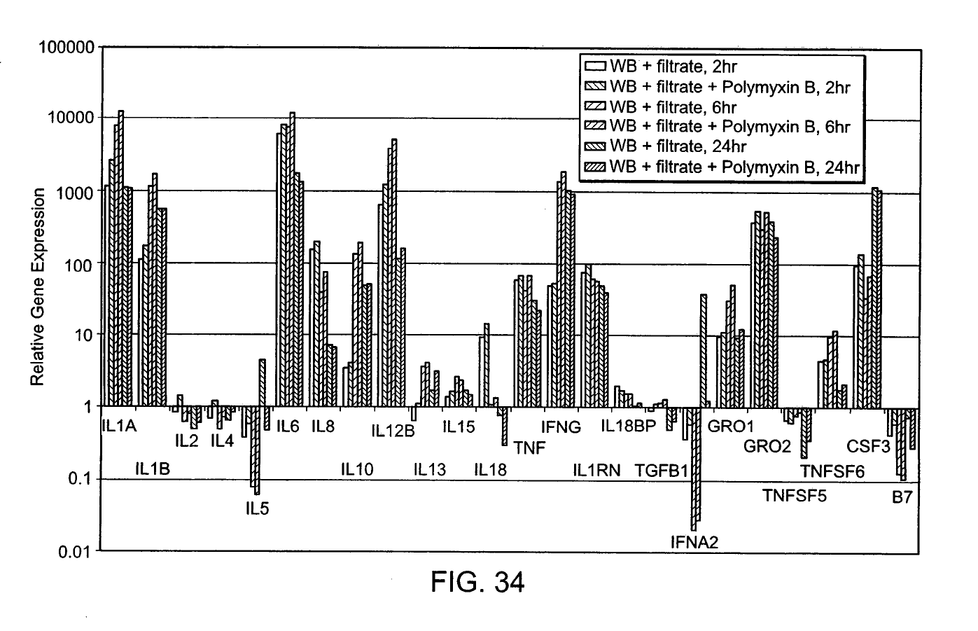

FIG. 34 is similar to FIG. 33, but here the compared responses are to stimuli from E. coli filtrate alone and from E. coli filtrate to which has been added polymyxin B, an antibiotic known to bind to lipopolysaccharide (LPS). An examination of the response of IL1B, for example, shows that presence of polymyxin B did not affect the response of the locus to E. coli filtrate, thereby indicating that LPS does not appear to be a factor in the response of IL1B to E. coli filtrate.

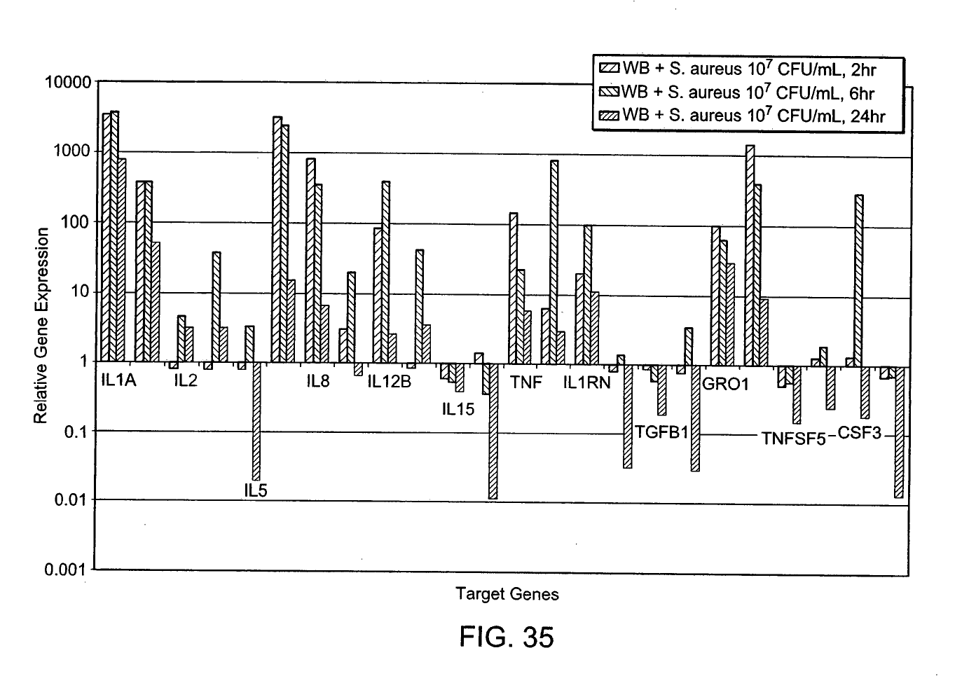

FIG. 35 illustrates the responses of the Inflammation 48A loci over time of whole blood to a stimulus of S. aureus (with a concentration just after administration of 10⁷ CFU/mL) monitored at 2, 6, and 24 hours after administration. It can be seen that response over time can involve both direction and magnitude of change in expression. (See for example, IL5 and IL18.)

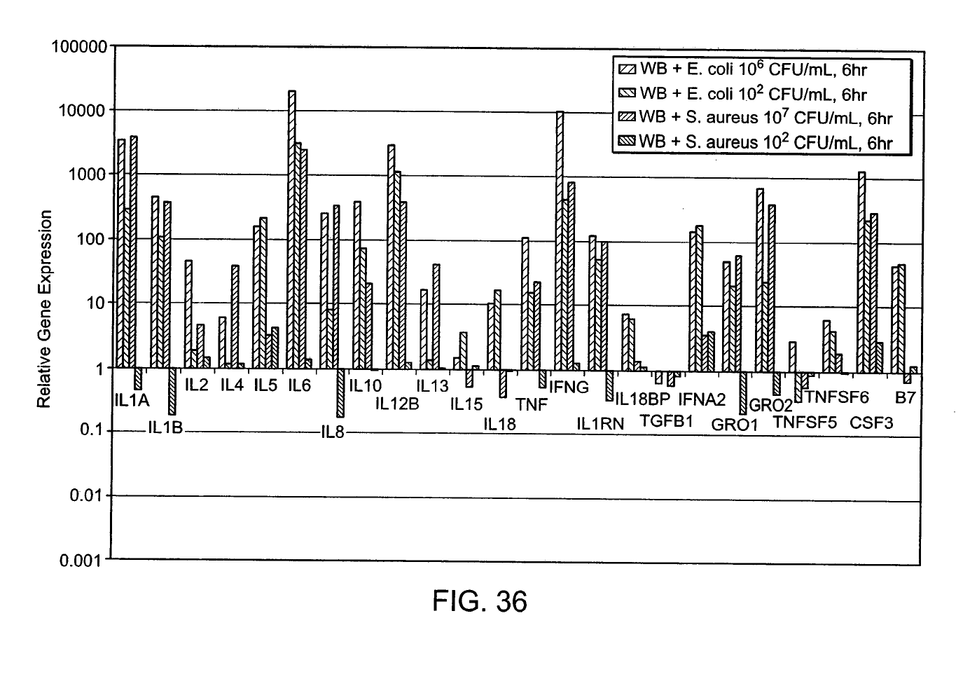

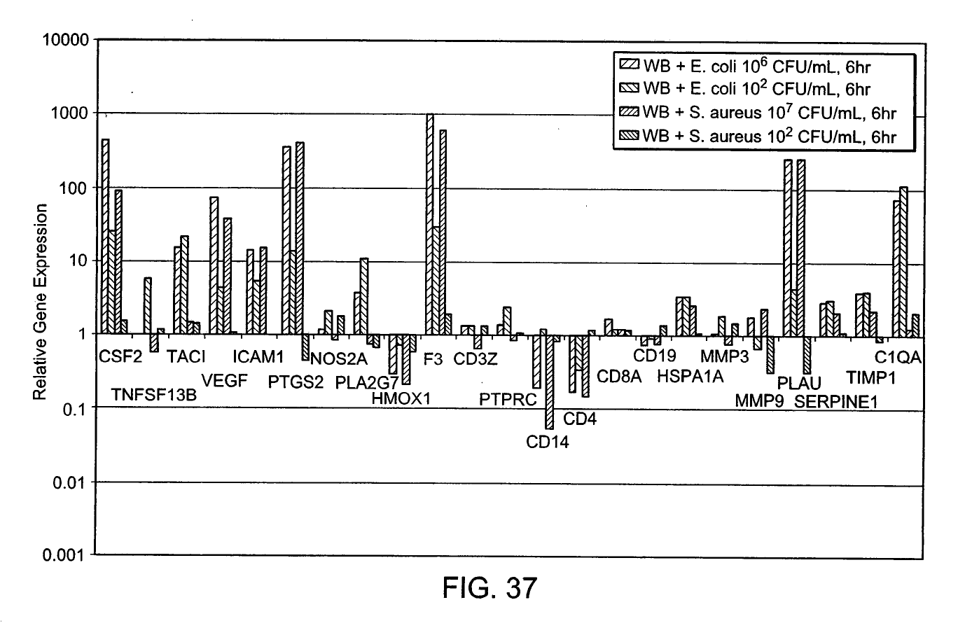

FIGS. 36 and 37 show the responses, of the Inflammation 48A and 48B loci respectively, monitored at 6 hours to stimuli from E. coli (at concentrations of 10⁶ and 10² CFU/mL immediately after administration) and from S. aureus (at concentrations of 10⁷ and 10² CFU/mL immediately after administration). It can be seen, among other things, that in various loci, such as B7 (FIG. 36), TACI, PLA2G7, and C1QA (FIG. 37), E. coli produces a much more pronounced response than S. aureus. The data suggest strongly that Gene Expression Profiles can be used to identify with high sensitivity the presence of gram negative bacteria and to discriminate against gram positive bacteria.

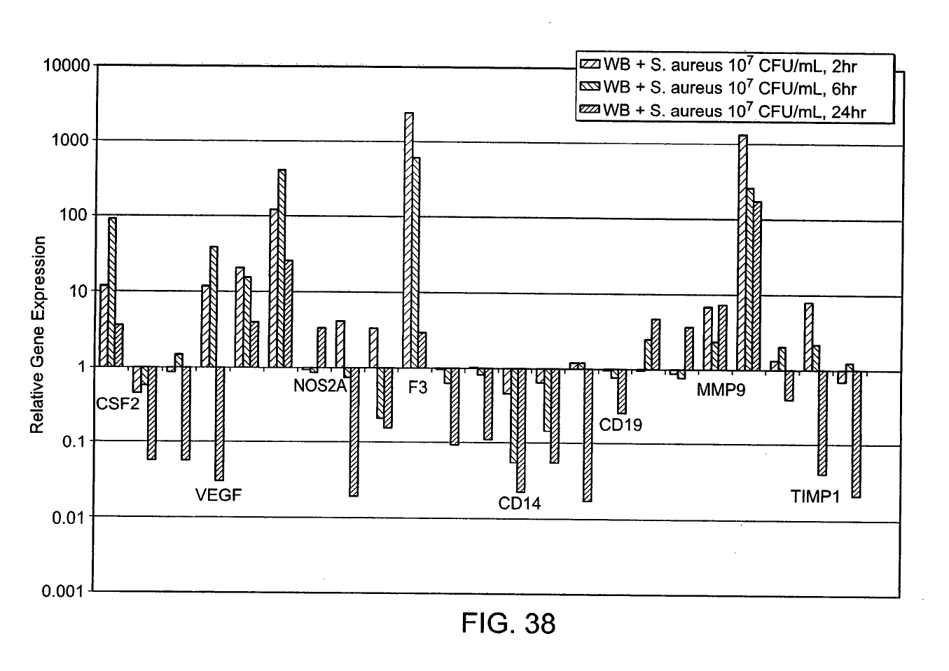

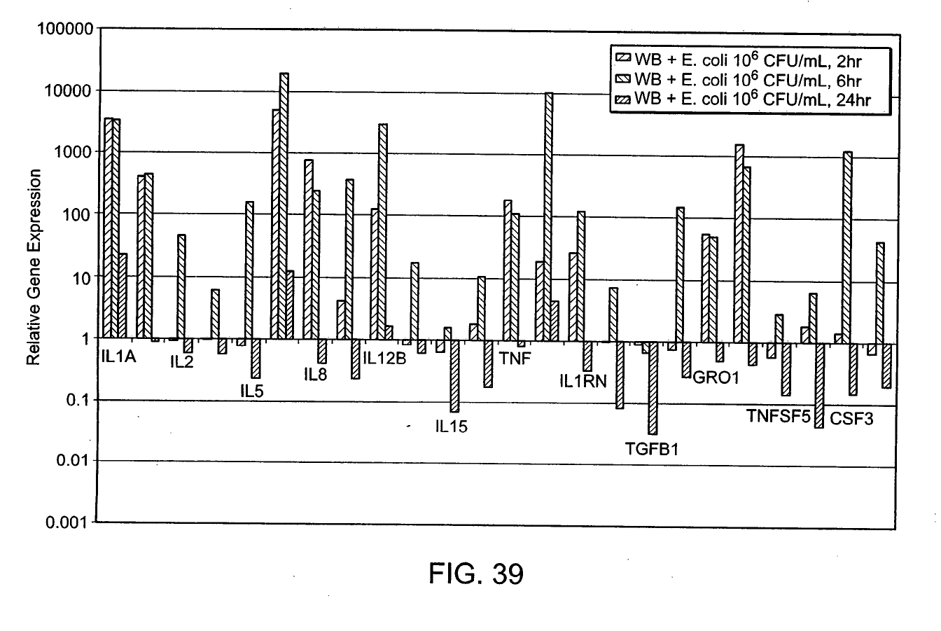

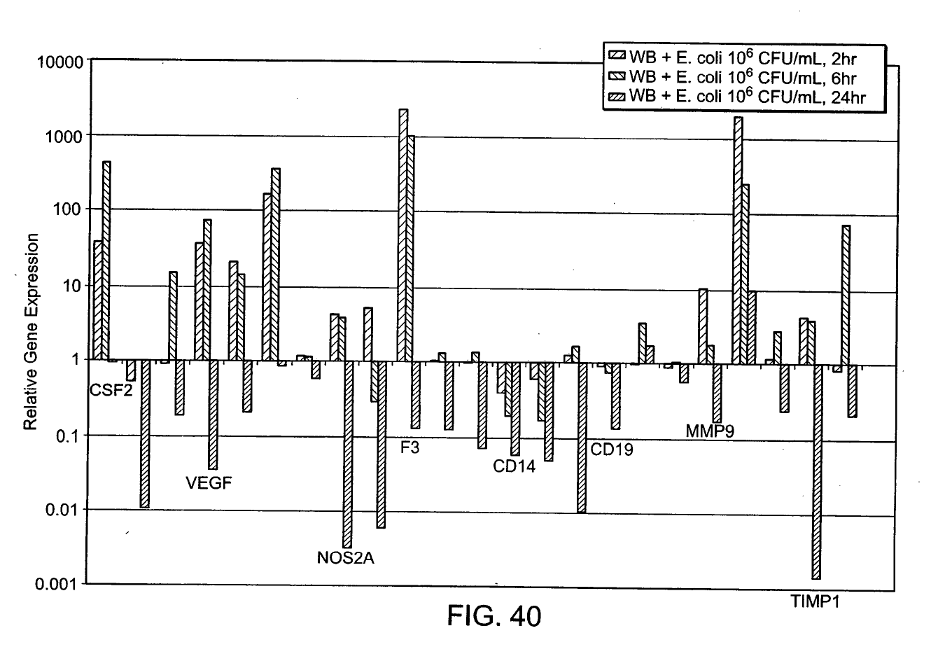

FIGS. 38 and 39 show the responses, of the Inflammation 48B and 48A loci respectively, monitored 2, 6, and 24 hours after administration, to stimuli of high concentrations of S. aureus and E. coli respectively (at respective concentrations of 10⁷ and 10⁶ CFU/mL immediately after administration). The responses over time at many loci involve changes in magnitude and direction. FIG. 40 is similar to FIG. 39, but shows the responses of the Inflammation 48B loci.

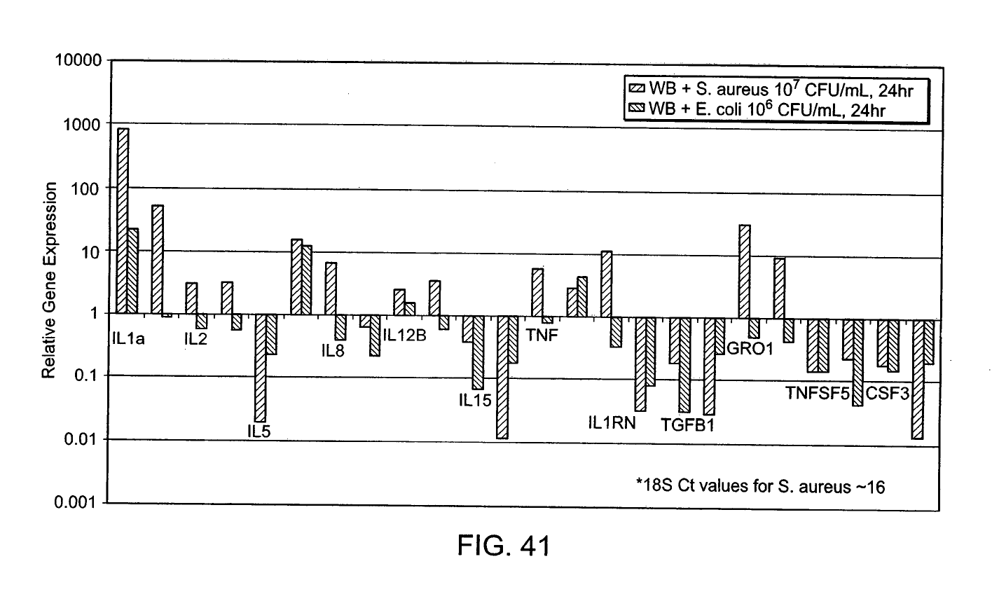

FIG. 41 similarly shows the responses of the Inflammation 48A loci monitored at 24 hours after administration to stimuli high concentrations of S. aureus and E. coli respectively (at respective concentrations of 10⁷ and 10⁶ CFU/mL immediately after administration). As in the case of FIGS. 20 and 21, responses at some loci, such as GRO1 and GRO2, discriminate between type of infection.

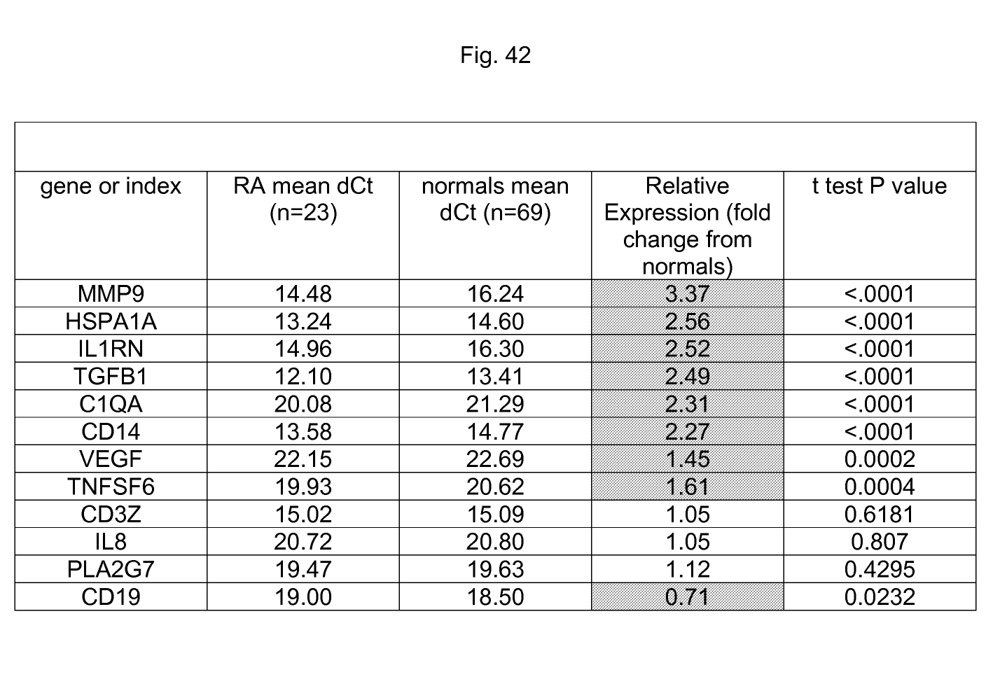

FIG. 42 illustrates application of a statistical T-test to identify potential members of a signature gene expression panel that is capable of distinguishing between normal subjects and subjects suffering from unstable rheumatoid arthritis. The grayed boxes show genes that are individually highly effective (t test P values noted in the box to the right in each case) in distinguishing between the two sets of subjects, and thus indicative of potential members of a signature gene expression panel for rheumatoid arthritis.

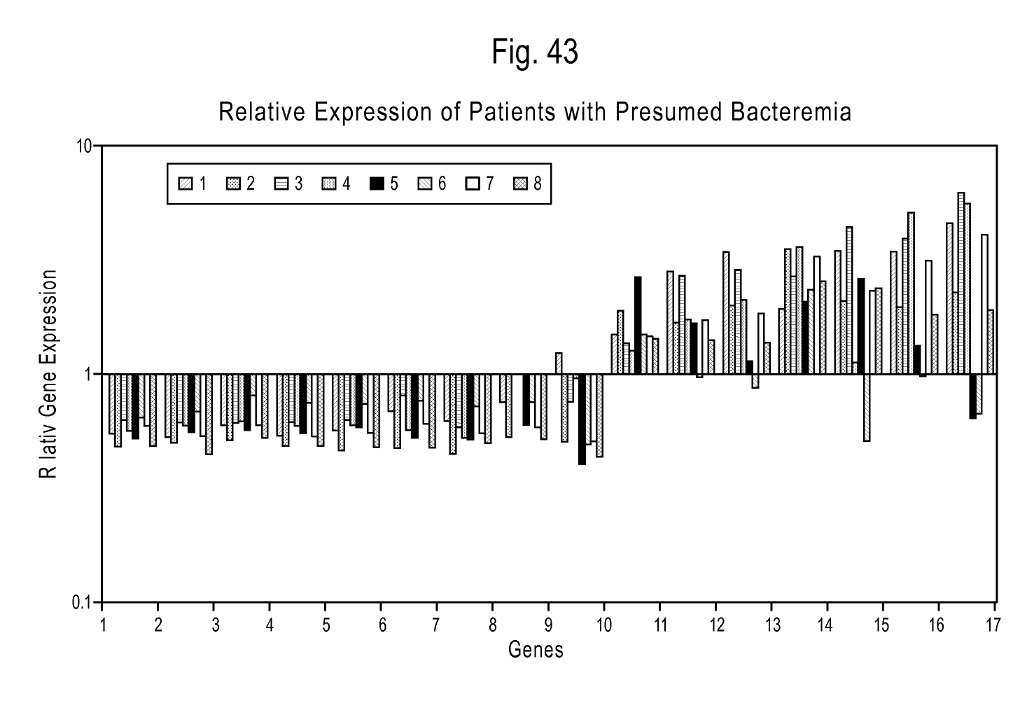

FIG. 43 illustrates, for a panel of 17 genes, the expression levels for 8 patients presumed to have bacteremia. The data are suggestive of the prospect that patients with bacteremia have a characteristic pattern of gene expression.

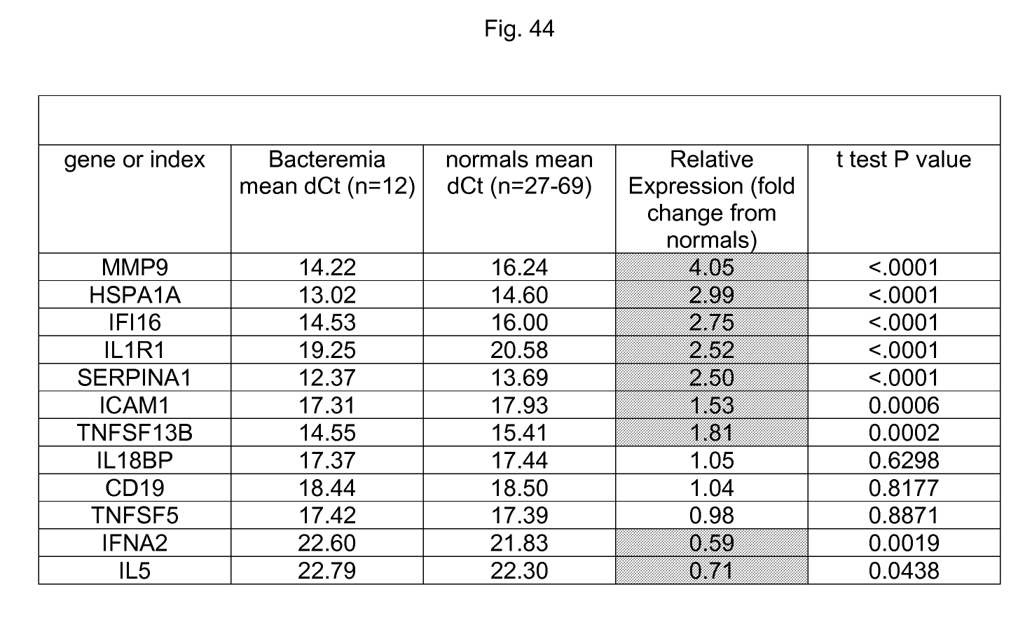

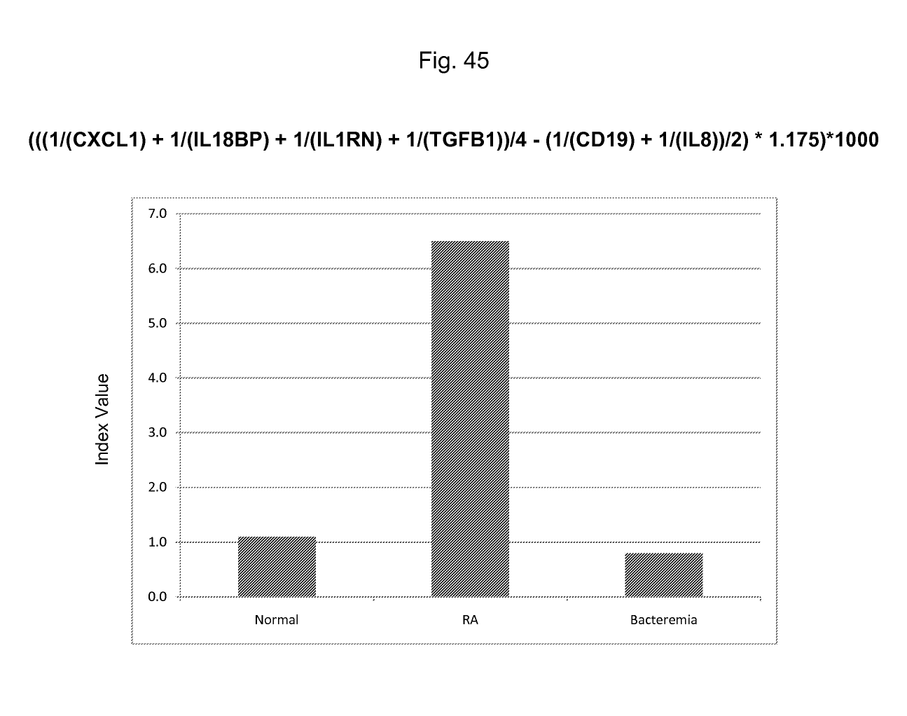

FIG. 44 illustrates application of a statistical T-test to identify potential members of a signature gene expression panel that is capable of distinguishing between normal subjects and subjects suffering from bacteremia. The grayed boxes show genes that are individually highly effective (t test P values noted in the box to the right in each case) in distinguishing between the two sets of subjects, and thus indicative of potential members of a signature gene expression panel for bacteremia.

FIG. 45 illustrates application of an algorithm (shown in the figure), providing an index pertinent to rheumatoid arthritis (RA) as applied respectively to normal subjects, RA patients, and bacteremia patients. The index easily distinguishes RA subjects from both normal subjects and bacteremia subjects.

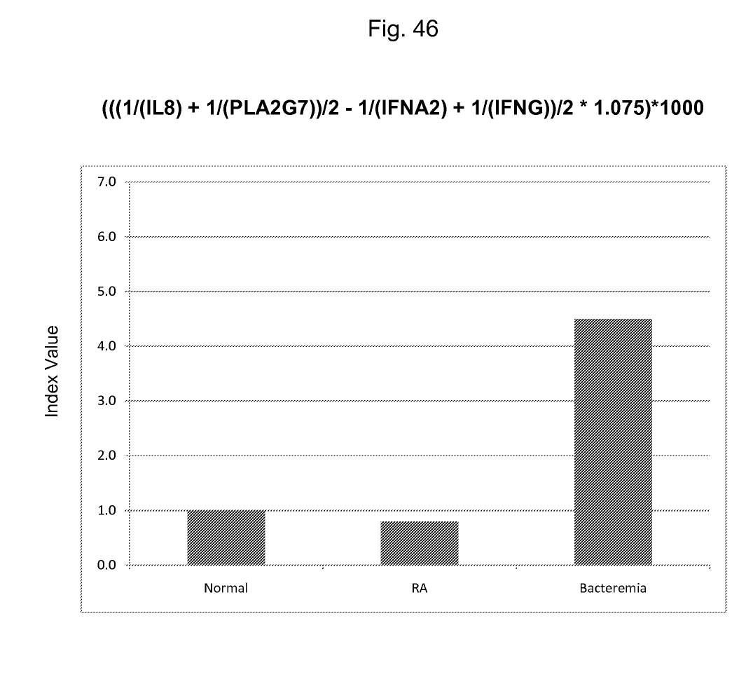

FIG. 46 illustrates application of an algorithm (shown in the figure), providing an index pertinent to bacteremia as applied respectively to normal subjects, rheumatoid arthritis patients, and bacteremia patients. The index easily distinguishes bacteremia subjects from both normal subjects and rheumatoid arthritis subjects.

These data support our conclusion that Gene Expression Profiles with sufficient precision and calibration as described herein (1) can determine subsets of individuals with a known biological condition; (2) may be used to monitor the response of patients to therapy; (3) may be used to assess the efficacy and safety of therapy; and (4) may used to guide the medical management of a patient by adjusting therapy to bring one or more relevant Gene Expression Profiles closer to a target set of values, which may be normative values or other desired or achievable values. We have shown that Gene Expression Profiles may provide meaningful information even when derived from ex vivo treatment of blood or other tissue. We have also shown that Gene Expression Profiles derived from peripheral whole blood are informative of a wide range of conditions neither directly nor typically associated with blood.

Furthermore, in embodiments of the present invention, Gene Expression Profiles can also be used for characterization and early identification (including pre-symptomatic states) of infectious disease, such as sepsis. This characterization includes discriminating between infected and uninfected individuals, bacterial and viral infections, specific subtypes of pathogenic agents, stages of the natural history of infection (e.g., early or late), and prognosis. Use of the algorithmic and statistical approaches discussed above to achieve such identification and to discriminate in such fashion is within the scope of various embodiments herein.

TABLE 1
Master Infectious Disease or Inflammatory Conditions Related to Infectious Disease Gene
Expression Panel

| Symbol | Name | Classification | Description |
|---|---|---|---|
| ABCC1 | ATP-binding | membrane | AKA MRP1, ABC29: Multispecific organic |
| | cassette, sub-family | transporter | anion membrane transporter; over expression |
| | C, member 1 | | confers tissue protection against a wide variety |
| | | | of xenobiotics due to their removal from the |
| | | | cell. |
| ABL1 | V-abl Abelson | oncogene | Cytoplasmic and nuclear protein tyrosine kinase |
| | murine leukemia | | implicated in cell differentiation, division, |
| | viral oncogene | | adhesion and stress response. Alterations of |
| | homolog 1 | | ABL1 lead to malignant transformations. |
| ACPP | Acid phosphatase, | phosphatase | AKA PAP: Major phosphatase of the prostate; |
| | prostate | | synthesized under androgen regulation; secreted |
| | | | by the epithelial cells of the prostrate |
| ACTB | Actin, beta | Cell Structure | Actins are highly conserved proteins that are |
| | | | involved in cell motility, structure and integrity. |
| | | | ACTB is one of two non-muscle cytoskeletal |
| | | | actins. Site of action for cytochalasin B effects |
| | | | on cell motility. |
| ADAMTS1 | Disintegrin-like and | Protease | AKA METH1; Inhibits endothelial cell |
| | metalloprotease | | proliferation; may inhibit angiogenesis; |
| | (reprolysin type) | | expression may be associated with development |
| | with | | of cancer cachexia. |
| | thrombospondin | | |
| | type 1 motif, 1 | | |
| AHR | Aryl hydrocarbon | Metabolism | Increases expression of xenobiotic metabolizing |
| | receptor | Receptor/Transcription | enzymes (ie P450) in response to binding of |
| | | Factor | planar aromatic hydrocarbons |
| ALB | Albumin | Liver Health | Carrier protein found in blood serum, |
| | | Indicator | synthesized in the liver, downregulation linked |
| | | | to decreased liver function/health |
| APAF1 | Apoptotic Protease | protease activating | Cytochrome c binds to APAF1, triggering |
| | Activating Factor 1 | peptide | activation of CASP3, leading to apoptosis. May |
| | | | also facilitate procaspase 9 auto activation. |
| ARG2 | Arginase II | Enzyme/redox | Catalyzes the hydrolysis of arginine to ornithine |
| | | | and urea; may play a role in down regulation of |
| | | | nitric oxide synthesis |
| B7 | B7 protein | cell signaling and | Regulatory protein that may be associated with |
| | | activation | lupus |
| BAD | BCL2 Agonist of | membrane protein | Heterodimerizes with BCLX and counters its |
| | Cell Death | | death repressor activity. This displaces BAX |
| | | | and restores its apoptosis-inducing activity. |
| BAK1 | BCL2- | membrane protein | In the presence of an appropriate stimulus BAK |
| | antagonist/killer 1 | | 1 accelerates programmed cell death by binding |
| | | | to, and antagonizing the repressor BCL2 or its |
| | | | adenovirus homolog e1b 19k protein. |
| BAX | BCL2 associated X | apoptosis | Accelerates programmed cell death by binding |
| | protein | induction-germ | to and antagonizing the apoptosis repressor |
| | | cell development | BCL2; may induce caspase activation |
| BCL2 | B-cell CLL/ | apoptosis Inhibitor- | Blocks apoptosis by interfering with the |
| | lymphoma 2 | cell cycle control- | activation of caspases |
| | | oncogenesis | |
| BCL2L1 | BCL2-like 1 (long | membrane protein | Dominant regulator of apoptotic cell death. The |
| | form) | | long form displays cell death repressor activity, |
| | | | whereas the short isoform promotes apoptosis. |
| | | | BCL2L1 promotes cell survival by regulating |
| | | | the electrical and osmotic homeostasis of |
| | | | mitochondria. |
| BID | BH3-Interacting | | Induces ice-like proteases and apoptosis. |
| | Death Domain | | Counters the protective effect of bcl-2 (by |
| | Agonist | | similarity). Encodes a novel death agonist that |
| | | | heterodimerizes with either agonists (BAX) or |
| | | | antagonists (BCL2). |
| BIK | BCL2-Interacting | | Accelerates apoptosis. Binding to the apoptosis |
| | Killer | | repressors BCL2L1, bhrf1, BCL2 or its |
| | | | adenovirus homolog e1b 19k protein suppresses |
| | | | this death-promoting activity. |
| BIRC2 | Baculoviral IAP | apoptosis | May inhibit apoptosis by regulating signals |
| | Repeat-Containing 2 | suppressor | required for activation of ICE-like proteases. |
| | | | Interacts with TRAF1 and TRAF2. Cytoplasmic |
| BIRC3 | Baculoviral IAP | apoptosis | Apoptotic suppressor. Interacts with TRAF1 |
| | Repeat-Containing 3 | suppressor | and TRAF2. Cytoplasmic |
| BIRC5 | Baculoviral IAP | apoptosis Inhibitor | AKA Survivin; API4: May counteract a default |
| | repeat-containing 5 | | induction of apoptosis in G2/M phase of cell |
| | | | cycle; associates with microtubules of the |
| | | | mitotic spindle during apoptosis |
| BSG | Basignin | signal | Member of Ig superfamily; tumor cell-derived |
| | | transduction- | collagenase stimulatory factor; stimulates |
| | | peripheral plasma | matrix metalloproteinase synthesis in fibroblasts |
| | | membrane protein | |
| BPI | Bactericidal/permeability- | Membrane-bound | LPS binding protein; cytotoxic for many gram |
| | increasing | protease | negative organisms; found in myeloid cells |
| | protein | | |
| C1QA | Complement | Proteinase/ | Serum complement system; forms C1 complex |
| | component 1, q | Proteinase | with the proenzymes c1r and c1s |
| | subcomponent, | Inhibitor | |
| | alpha polypeptide | | |
| CALCA | Calcitonin/calcitonin- | Cell-signaling and | AKA CALC1; Promotes rapid incorporation of |
| | related | activation | calcium into bone |
| | polypeptide, alpha | | |
| CASP1 | Caspase 1 | proteinase | Activates IL1B; stimulates apoptosis |
| CASP3 | Caspase 3 | Proteinase/ | Involved in activation cascade of caspases |
| | | Proteinase | responsible for apoptosis - cleaves CASP6, |
| | | Inhibitor | CASP7, CASP9 |
| CASP9 | Caspase 9 | proteinase | Binds with APAF1 to become activated; cleaves |
| | | | and activates CASP3 |
| CCL3 | Chemokine (C-C | Cytokines- | AKA: MIP1-alpha; monkine that binds to |
| | motif) ligand 3 | chemokines- | CCR1, CCR4 and CCR5; major HIV- |
| | | growth factors | suppressive factor produced by CD8 cells. |
| CCNA2 | Cyclin A2 | cyclin | Drives cell cycle at G1/S and G2/M phase; |
| | | | interacts with cdk2 and cdc2 |
| CCNB1 | Cyclin B1 | cyclin | Drives cell cycle at G2/M phase; complexes |
| | | | with cdc2 to form mitosis promoting factor |
| CCND1 | Cyclin D1 | cyclin | Controls cell cycle at G1/S (start) phase; |
| | | | interacts with cdk4 and cdk6; has oncogene |
| | | | function |
| CCND3 | Cyclin D3 | cyclin | Drives cell cycle at G1/S phase; expression |
| | | | rises later in G1 and remains elevated in S |
| | | | phase; interacts with cdk4 and cdk6 |
| CCNE1 | Cyclin E1 | cyclin | Drives cell cycle at G1/S transition; major |
| | | | downstream target of CCND1; cdk2-CCNE1 |
| | | | activity required for centrosome duplication |
| | | | during S phase; interacts with RB |
| CCR1 | chemokine (C-C | Chemokine | A member of the beta chemokine receptor |
| | motif) receptor 1 | receptor | family (seven transmembrane proteins). Binds |
| | | | SCYA3/MIP-1a, SCYA5/RANTES, MCP-3, |
| | | | HCC-1, 2, and 4, and MPIF-1. Plays role in |
| | | | dendritic cell migration to inflammation sites |
| | | | and recruitment of monocytes. |
| CCR3 | chemokine (C-C | Chemokine | C-C type chemokine receptor (Eotaxin receptor) |
| | motif) receptor 3 | receptor | binds to Eotaxin, Eotaxin-3, MCP-3, MCP-4, |
| | | | SCYA5/RANTES and mip-1 delta thereby |
| | | | mediating intracellular calcium flux. |
| | | | Alternative co-receptor with CD4 for HIV-1 |
| | | | infection. Involved in recruitment of |
| | | | eosinophils. Primarily a Th2 cell chemokine |
| | | | receptor. |
| CCR5 | chemokine (C-C | Chemokine | Member of the beta chemokine receptor family |
| | motif) receptor 5 | receptor | (seven transmembrane proteins). Binds to |
| | | | SCYA3/MIP-1a and SCYA5/RANTES. |
| | | | Expressed by T cells and macrophages, and is |
| | | | an important co-receptor for macrophage-tropic |
| | | | virus, including HIV, to enter host cells. Plays a |
| | | | role in Th1 cell migration. Defective alleles of |
| | | | this gene have been associated with the HIV |
| | | | infection resistance. |
| CD14 | CD14 antigen | Cell Marker | LPS receptor used as marker for monocytes |
| CD19 | CD19 antigen | Cell Marker | AKA Leu 12; B cell growth factor |
| CD34 | CD34 antigen | Cell Marker | AKA: hematopoietic progenitor cell antigen. |
| | | | Cell surface antigen selectively expressed on |
| | | | human hematopoietic progenitor cells. |
| | | | Endothelial marker. |
| CD3Z | CD3 antigen, zeta | Cell Marker | T-cell surface glycoprotein |
| | polypeptide | | |
| CD4 | CD4 antigen (p55) | Cell Marker | Helper T-cell marker |
| CD44 | CD44 antigen | Cell Marker | Cell surface receptor for hyaluronate. Probably |
| | | | involved in matrix adhesion, lymphocyte |
| | | | activation and lymph node homing. |
| CD86 | CD 86 Antigen (cD | Cell signaling and | AKA B7-2; membrane protein found in B |
| | 28 antigen ligand) | activation | lymphocytes and monocytes; co-stimulatory |
| | | | signal necessary for T lymphocyte proliferation |
| | | | through IL2 production. |
| CD8A | CD8 antigen, alpha | Cell Marker | Suppressor T cell marker |
| | polypeptide | | |
| CDH1 | Cadherin 1, type 1, | cell-cell adhesion/ | AKA ECAD, UVO: Calcium ion-dependent cell |
| | E-cadherin | interaction | adhesion molecule that mediates cell to cell |
| | | | interactions in epithelial cells |
| CDH2 | Cadherin 2, type 1, | cell-cell adhesion/ | AKA NCAD, CDHN: Calcium-dependent |
| | N-cadherin | interaction | glycoprotein that mediates cell-cell interactions; |
| | | | may be involved in neuronal recognition |
| | | | mechanism |
| cdk2 | Cyclin-dependent | kinase | Associated with cyclins A, D and E; activity |
| | kinase 2 | | maximal during S phase and G2; CDK2 |
| | | | activation, through caspase-mediated cleavage |
| | | | of CDK inhibitors, may be instrumental in the |
| | | | execution of apoptosis following caspase |
| | | | activation |
| cdk4 | Cyclin-dependent | kinase | cdk4 and cyclin-D type complexes are |
| | kinase 4 | | responsible for cell proliferation during G1; |
| | | | inhibited by CDKN2A (p16) |
| CDKN1A | Cyclin-Dependent | tumor suppressor | May bind to and inhibit cyclin-dependent kinase |
| | Kinase Inhibitor 1A | | activity, preventing phosphorylation of critical |
| | (p21) | | cyclin-dependent kinase substrates and blocking |
| | | | cell cycle progression; activated by p53; tumor |
| | | | suppressor function |
| CDKN2A | Cyclin-dependent | cell cycle control- | AKA p16, MTS1, INK4: Tumor suppressor |
| | kinase inhibitor 2A | tumor suppressor | gene involved in a variety of malignancies; |
| | | | arrests normal diploid cells in late G1 |
| CDKN2B | Cyclin-Dependent | tumor suppressor | Interacts strongly with cdk4 and cdk6; role in |
| | Kinase Inhibitor 2B | | growth regulation but limited role as tumor |
| | (p15) | | suppressor |
| CHEK1 | Checkpoint, | | Involved in cell cycle arrest when DNA damage |
| | S. pombe | | has occurred, or unligated DNA is present; |
| | | | prevents activation of the cdc2-cyclin b |
| | | | complex |
| CLDN14 | Claudin 14 | | AKA DFNB29; Component of tight junction |
| | | | strands |
| COL1A1 | Collagen, type 1, | Tissue | AKA Procollagen; extracellular matrix protein; |
| | alpha 1 | Remodeling | implicated in fibrotic processes of damaged |
| | | | liver |
| COL7A1 | Type VII collagen, | collagen- | alpha 1 subunit of type VII collagen; may link |
| | alpha 1 | differentiation- | collagen fibrils to the basement membrane |
| | | extracellular | |
| | | matrix | |
| CRABP2 | Cellular Retinoic | retinoid binding- | Low molecular weight protein highly expressed |
| | Acid Binding | signal | in skin; thought to be important in RA-mediated |
| | Protein | transduction- | regulation of skin growth & differentiation |
| | | transcription | |
| | | regulation | |
| CRP | C-reactive protein | Acute phase | Acute phase protein |
| | | protein | |
| CSF2 | Granulocyte- | cytokines- | AKA GM-CSF; Hematopoietic growth factor; |
| | monocyte colony | chemokines- | stimulates growth and differentiation of |
| | stimulating factor | growth factors | hematopoietic precursor cells from various |
| | | | lineages, including granulocytes, macrophages, |
| | | | eosinophils, and erythrocytes |
| CSF3 | Colony stimulating | cytokines- | AKA GCSF controls production ifferentiation |
| | factor 3 | chemokines- | and function of granulocytes. |
| | (granulocyte) | growth factors | |
| CTGF | Connective Tissue | insulin-like | Member of family of peptides including serum- |
| | Growth Factor | growth factor- | induced immediate early gene products |
| | | differentiation- | expressed after induction by growth factors; |
| | | wounding | over expressed in fibrotic disorders |
| | | response | |
| CTNNA1 | Catenin, alpha 1 | cell adhesion | Binds cadherins and links them with the actin |
| | | | cytoskeleton |
| CX3CR1 | chemokine (C—X3—C) | Chemokine | CX3CR1 is an HIV coreceptor as well as a |
| | receptor 1 | receptor | leukocyte chemotactic/adhesion receptor for |
| | | | fractalkine. Natural killer cells predominantly |
| | | | express CX3CR1 and respond to fractalkine in |
| | | | both migration and adhesion. |
| CXCR4 | chemokine (C—X—C | Chemokine | Receptor for the CXC chemokine SDF1. Acts |
| | motif), receptor 4 | receptor | as a co-receptor with CD4 for lymphocyte- |
| | (fusin) | | tropic HIV-1 viruses. Plays role in B cell, Th2 |
| | | | cell and naive T cell migration. |
| CYP1A1 | Cytochrome P450 | Metabolism | Polycyclic aromatic hydrocarbon metabolism; |
| | 1A1 | Enzyme | monooxygenase |
| CYP1A2 | Cytochrome P450 | Metabolism | Polycyclic aromatic hydrocarbon metabolism; |
| | 1A2 | Enzyme | monooxygenase |
| CYP2C19 | Cytochrome P450 | Metabolism | Xenobiotic metabolism; monooxygenase |
| | 2C19 | Enzyme | |
| CYP2D6 | Cytochrome P450 | Metabolism | Xenobiotic metabolism; monooxygenase |
| | 2D6 | Enzyme | |
| CYP2E | Cytochrome P450 | Metabolism | Xenobiotic metabolism; monooxygenase; |
| | 2E1 | Enzyme | catalyzes formation of reactive intermediates |
| | | | from small organic molecules (i.e. ethanol, |
| | | | acetaminophen, carbon tetrachloride) |
| CYP3A4 | Cytochrome P450 | Metabolism | Xenobiotic metabolism; broad catalytic |
| | 3A4 | Enzyme | specificity, most abundantly expressed liver |
| | | | P450 |
| CXCL10 | Chemokine (C—X—C | Cytokines- | AKA: Gamma IP10; interferon inducible |
| | moif) ligand 10 | chemokines- | cytokine IP10; SCYB10; Ligand for CXCR3; |
| | | growth factors | binding causes stimulation of monocytes, NK |
| | | | cells; induces T cell migration |
| DAD1 | Defender Against | membrane protein | Loss of DAD1 protein triggers apoptosis |
| | Cell Death | | |
| DC13 | DC13 protein | | unknown function |
| DFFB | DNA Fragmentation | nuclease | Induces DNA fragmentation and chromatin |
| | Factor, 40-KD, | | condensation during apoptosis; can be activated |
| | Beta Subunit | | by CASP3 |
| DSG1 | Desmoglein 1 | membrane protein | Calcium-binding transmembrane glycoprotein |
| | | | involved in the interaction of plaque proteins |
| | | | and intermediate filaments mediating cell-cell |
| | | | adhesion. Interact with cadherins. |
| DTR | Diphtheria toxin | Cell signaling, | Thought to be involved in macrophage- |
| | receptor (heparin- | mitogen | mediated cellular proliferation. DTR is a potent |
| | binding epidermal | | mitogen and chemotactic factor for fibroblasts |
| | growth factor-like | | and smooth muscle cells, but not endothelial |
| | growth factor) | | cells. |
| DUSP1 | Dual Specificity | oxidative stress | Induced in human skin fibroblasts by |
| | Phosphatase | response-tyrosine | oxidative/heat stress & growth factors; de- |
| | | phosphatase | phosphorylates MAP kinase erk2; may play a |
| | | | role in negative regulation of cellular |
| | | | proliferation |
| ECE1 | Endothelin | Metalloprotease | Cleaves big endothelin 1 to endothelin 1 |
| | converting enzyme 1 | | |
| EDN1 | Endothelin 1 | Peptide hormone | AKA ET1; Endothelium-derived peptides; |
| | | | potent vasoconstrictor |
| EDR2 | Early Development | | The specific function in human cells has not yet |
| | Regulator 2 | | been determined. May be part of a complex |
| | | | that may regulate transcription during |
| | | | embryonic development. |
| EGR1 | Early growth | Transcription | AKA NGF1A; Regulates the transcription of |
| | response 1 | factor | genes involved in mitogenesis and |
| | | | differentiation |
| ELA2 | Elastase 2, | Protease | Modifies the functions of NK cells, monocytes |
| | neutrophil | | and granulocytes |
| EPHX1 | Epoxide hydrolase | Metabolism | Catalyzes hydrolysis of reactive epoxides to |
| | 1, microsomal | Enzyme | water soluble dihydrodiols |
| | (xenobiotic) | | |
| ERBB2 | v-erb-b2 | Oncogene | Oncogene. Over expression of ERBB2 confers |
| | erythroblastic | | Taxol resistance in breast cancers. Belongs to |
| | leukemia viral | | the EGF tyrosine kinase receptor family. Binds |
| | oncogene homolog 2 | | gp130 subunit of the IL6 receptor in an IL6 |
| | | | dependent manner. An essential component of |
| | | | IL-6 signaling through the MAP kinase |
| | | | pathway. |
| ERBB3 | v-erb-b2 | Oncogene | Oncogene. Over expressed in mammary |
| | Erythroblastic | | tumors. Belongs to the EGF tyrosine kinase |
| | Leukemia Viral | | receptor family. Activated through neuregulin |
| | Oncogene Homolog 3 | | and ntak binding. |
| ESR1 | Estrogen Receptor 1 | Receptor/ | ESR1 is a ligand-activated transcription factor |
| | | Transcription | composed of several domains important for |
| | | Factor | hormone binding, DNA binding, and activation |
| | | | of transcription. |
| F3 | F3 | Enzyme/Redox | AKA thromboplastin, Coagulation Factor 3; cell |
| | | | surface glycoprotein responsible for coagulation |
| | | | catalysis |
| FADD | Fas (TNFRSF6)- | co-receptor | Apoptotic adaptor molecule that recruits |
| | associated via death | | caspase-8 or caspase-10 to the activated fas |
| | domain | | (cd95) or tnfr-1 receptors; this death-inducing |
| | | | signaling complex performs CASP8 proteolytic |
| | | | activation |
| FAP | Fibroblast activation | Liver Health | Expressed in cancer stroma and wound healing |
| | protein, □ | Indicator | |
| FCGR1A | Fc fragment of lgG, | Membrane protein | Membrane receptor of CD64; found in |
| | high affinity | | monocytes, macrophages and neutrophils |
| | receptor IA | | |
| FGF18 | Fibroblast Growth | Growth Factor | Involved in a variety of biological processes, |
| | Factor 18 | | including embryonic development, cell growth, |
| | | | morphogenesis, tissue repair, tumor growth, and |
| | | | invasion. |
| FGF7 | Fibroblast growth | growth factor- | aka KGF; Potent mitogen for epithelial cells; |
| | factor 7 | differentiation- | induced after skin injury |
| | | wounding | |
| | | response-signal | |
| | | transduction | |
| FLT1 | Fms-related tyrosine | | AKA VEGFR1; FRT; Receptor for VEGF; |
| | kinase 1 (vascular | | involved in vascular development and |
| | endothelial growth | | regulation of vascular permeability |
| | factor/vascular | | |
| | permeability factor | | |
| | receptor) | | |
| FN1 | Fibronectin | cell adhesion- | Major cell surface glycoprotein of many |
| | | motility-signal | fibroblast cells; thought to have a role in cell |
| | | transduction | adhesion, morphology, wound healing & cell |
| | | | motility |
| FTL | Ferritin, light | Iron Chelator | Intracellular, iron storage protein |
| | polypeptide | | |
| FOLH1 | Folate Hydrolase | hydrolase | AKA PSMA, GCP2: Expressed in normal and |
| | | | neoplastic prostate cells; membrane bound |
| | | | glycoprotein; hydrolyzes folate and is an N- |
| | | | acetylated a-linked acidic dipeptidase |
| FOS | v-fos FBJ murine | transcription | Proto-oncoprotein acting with JUN, stimulates |
| | osteosarcoma virus | factor- | transcription of genes with AP-1 regulatory |
| | oncogene homolog | inflammatory | sites; in some cases FOS expression is |
| | | response-cell | associated with apoptotic cell death |
| | | growth & | |
| | | maintenance | |
| G6PC | glucose-6- | Glucose-6- | Catalyzes the final step in the gluconeogenic |
| | phosphatase, | phosphatase/Glycogen | and glycogenolytic pathways. Stimulated by |
| | catalytic | metabolism | glucocorticoids and strongly inhibited by |
| | | | insulin. Over expression (in conjunction with |
| | | | PCK1 over expression) leads to increased |
| | | | hepatic glucose production. |
| GADD45A | Growth Arrest and | cell cycle-DNA | Transcriptionally induced following stressful |
| | DNA-damage- | repair-apoptosis | growth arrest conditions & treatment with DNA |
| | inducible alpha | | damaging agents; binds to PCNA affecting it's |
| | | | interaction with some cell division protein |
| | | | kinase |
| GCG | glucagon | pancreatic/peptide | Pancreatic hormone which counteracts the |
| | | hormone | glucose-lowering action of insulin by |
| | | | stimulating glycogenolysis and |
| | | | gluconeogenesis. Under expression of glucagon |
| | | | is preferred. Glucagon-like peptide (GLP-1) |
| | | | proposed for type 2 diabetes treatment inhibits |
| | | | glucag |
| GCGR | glucagon receptor | glucagon receptor | Expression of GCGR is strongly unregulated by |
| | | | glucose. Deficiency or imbalance could play a |
| | | | role in NIDDM. Has been looked as a potential |
| | | | for gene therapy. |
| GFPT1 | glutamine-fructose- | Glutamine | The rate limiting enzyme for glucose entry into |
| | 6-phosphate | amidotransferase | the hexosamine biosynthetic pathway (HBP). |
| | transaminase 1 | | Over expression of GFA in muscle and adipose |
| | | | tissue increases products of the HBP which are |
| | | | thought to cause insulin resistance (possibly |
| | | | through defects to glucose |
| GJA1 | gap junction protein, | | AKA CX43; Protein component of gap |
| | alpha 1, 43 kD | | junctions; major component of gap junctions in |
| | | | the heart; may be important in synchronizing |
| | | | heart contractions and in embryonic |
| | | | development |
| GPR9 | G protein-coupled | Chemokine | CXC chemokine receptor binds to SCYB10/IP- |
| | receptor 9 | receptor | 10, SCYB9/MIG, and SCYB11/I-TAC. |
| | | | Binding of chemokines to GPR9 results in |
| | | | integrin activation, cytoskeletal changes and |
| | | | chemotactic migration. Prominently expressed |
| | | | in in vitro cultured effector/memory T cells and |
| | | | plays a role in Th1 cell migration. |
| GRO1 | GRO1 oncogene | cytokines- | AKA SCYB1; chemotactic for neutrophils |
| | (melanoma growth | chemokines- | |
| | stimulating activity, | growth factors | |
| | alpha) | | |
| GRO2 | GRO2 oncogene | cytokines- | AKA MIP2, SCYB2; Macrophage |
| | | chemokines- | inflammatory protein produced by monocytes |
| | | growth factors | and neutrophils |
| GSR | Glutathione | Oxidoreductase | AKA GR; GRASE; Maintains high levels of |
| | reductase 1 | | reduced glutathione in the cytosol |
| GST | Glutathione S- | Metabolism | Catalyzes glutathione conjugation to metabolic |
| | transferase | Enzyme | substrates to form more water-soluble, |
| | | | excretable compounds; primer-probe set |
| | | | nonspecific for all members of GST family |
| GSTA1 and | Glutathione S- | Metabolism | Catalyzes glutathione conjugation to metabolic |
| A2 | transferase 1A1/2 | Enzyme | substrates to form more water-soluble, |
| | | | excretable compounds |
| GSTM1 | Glutathione S- | Metabolism | Catalyzes glutathione conjugation to metabolic |
| | transferase M1 | Enzyme | substrates to form more water-soluble, |
| | | | excretable compounds |
| GSTT1 | Glutathione-S- | metabolism | Catalyzes the conjugation of reduced |
| | Transferase, theta 1 | | glutathione to a wide number of exogenous and |
| | | | endogenous hydrophobic electrophiles; has an |
| | | | important role in human carcinogenesis |
| GYS1 | glycogen synthase 1 | Transferase/Glycogen | A key enzyme in the regulation of glycogen |
| | (muscle) | metabolism | synthesis in the skeletal muscles of humans. |
| | | | Typically stimulated by insulin, but in NIDDM |
| | | | individuals GS is shown to be completely |
| | | | resistant to insulin stimulation (decreased |
| | | | activity and activation in muscle) |
| GZMB | Granzyme B | Proteinase/Proteinase | AKA CTLA1; Necessary for target cell lysis in |
| | | Inhibitor | cell-mediated immune responses. Crucial for |
| | | | the rapid induction of target cell apoptosis by |
| | | | cytotoxic T cells. Inhibition of the GZMB- |
| | | | IGF2R (receptor for GZMB) interaction |
| | | | prevented GZMB cell surface binding, uptake, |
| | | | and the induction of apoptosis. |
| HIF1A | Hypoxia-inducible | Transcription | AKA MOP1; ARNT interacting protein; |
| | factor 1, alpha | factor | mediates the transcription of oxygen regulated |
| | subunit | | genes; induced by hypoxia |
| HK2 | hexokinase 2 | hexokinase | Phosphorylates glucose into glucose-6- |
| | | | phosphate. NIDDM patients have lower HK2 |
| | | | activity which may contribute to insulin |
| | | | resistance. Similar action to GCK. |
| HLA-DRB1 | Major | Histocompatibility | Binds antigen for presentation to CD4+ cells |
| | histocompatibility | | |
| | complex, class II, | | |
| | DR beta 1 | | |
| HMGIY | High mobility group | DNA binding- | Potential oncogene with MYC binding site at |
| | protein, isoforms I | transcriptional | promoter region; involved in the transcription |
| | and Y | regulation- | regulation of genes containing, or in close |
| | | oncogene | proximity to a+t-rich regions |
| HMOX1 | Heme oxygenase | Enzyme/Redox | Endotoxin inducible |
| | (decycling) 1 | | |
| HSPA1A | Heat shock protein | Cell Signaling and | heat shock protein 70 kDa; Molecular |
| | 70 | activation | chaperone, stabilizes AU rich mRNA |
| ICAM1 | Intercellular | Cell Adhesion/ | Endothelial cell surface molecule; regulates cell |
| | adhesion molecule 1 | Matrix Protein | adhesion and trafficking, unregulated during |
| | | | cytokine stimulation |
| IFI16 | gamma interferon | cell signaling and | Transcriptional repressor |
| | inducible protein | activation | |
| | 16 | | |
| IFNA2 | Interferon, alpha 2 | cytokines- | interferon produced by macrophages with |
| | | chemokines-growth | antiviral effects |
| | | factors | |
| IFNG | Interferon, Gamma | Cytokines/ | Pro- and anti-inflammatory activity; TH1 |
| | | Chemokines/ | cytokine; nonspecific inflammatory mediator; |
| | | Growth Factors | produced by activated T-cells. |
| IGF1R | Insulin-like growth | cytokines- | Mediates insulin stimulated DNA synthesis; |
| | factor 1 receptor | chemokines- | mediates IGF1 stimulated cell proliferation and |
| | | growth factors | differentiation |
| IGFBP3 | Insulin-like growth | | AKA IBP3; Expressed by vascular endothelial |
| | factor binding | | cells; may influence insulin-like growth factor |
| | protein 3 | | activity |
| IL10 | Interleukin 10 | cytokines- | Anti-inflammatory; TH2; suppresses production |
| | | chemokines-growth | of proinflammatory cytokines |
| | | factors | |
| IL12B | Interleukin 12 p40 | cytokines- | Proinflammatory; mediator of innate immunity, |
| | | chemokines-growth | TH1 cytokine, requires co-stimulation with IL- |
| | | factors | 18 to induce IFN-g |
| IL13 | Interleukin 13 | Cytokines/ | Inhibits inflammatory cytokine production |
| | | Chemokines/ | |
| | | Growth Factors | |
| IL15 | Interleukin 15 | Cytokines/ | Proinflammatory; mediates T-cell activation, |
| | | Chemokines/ | inhibits apoptosis, synergizes with IL-2 to |
| | | Growth Factors | induce IFN-g and TNF-a |
| IL18 | Interleukin 18 | cytokines- | Proinflammatory, TH1, innate and acquired |
| | | chemokines-growth | immunity, promotes apoptosis, requires co- |
| | | factors | stimulation with IL-1 or IL-2 to induce TH1 |
| | | | cytokines in T- and NK-cells |
| IL18BP | IL-18 Binding | cytokines- | Implicated in inhibition of early TH1 cytokine |
| | Protein | chemokines-growth | responses |
| | | factors | |
| IL18RI | Interleukin 19 | Membrane protein | Receptor for interleukin 18; binding the agonist |
| | receptor 1 | | leads to activation of NFKB-B; belongs to IL1 |
| | | | family but does not bind IL1A or IL1B. |
| IL1A | Interleukin 1, alpha | cytokines- | Proinflammatory; constitutively and inducibly |
| | | chemokines-growth | expressed in variety of cells. Generally |
| | | factors | cytosolic and released only during severe |
| | | | inflammatory disease |
| IL1B | Interleukin 1, beta | cytokines- | Proinflammatory; constitutively and inducibly |
| | | chemokines-growth | expressed by many cell types, secreted |
| | | factors | |
| IL1R1 | interleukin 1 | Cell signaling and | AKA: CD12 or IL1R1RA; Binds all three forms |
| | receptor, type I | activation | of interleukin-1 (IL1A, IL1B and IL1RA). |
| | | | Binding of agonist leads to NFKB activation |
| IL1RN | Interleukin 1 | Cytokines/ | IL1 receptor antagonist; Anti-inflammatory; |
| | Receptor | Chemokines/ | inhibits binding of IL-1 to IL-1 receptor by |
| | Antagonist | Growth Factors | binding to receptor without stimulating IL-1- |
| | | | like activity |
| IL2 | Interleukin 2 | Cytokines/ | T-cell growth factor, expressed by activated T- |
| | | Chemokines/ | cells, regulates lymphocyte activation and |
| | | Growth Factors | differentiation; inhibits apoptosis, TH1 cytokine |
| IL4 | Interleukin 4 | Cytokines/ | Anti-inflammatory; TH2; suppresses |
| | | Chemokines/ | proinflammatory cytokines, increases |
| | | Growth Factors | expression of IL-1RN, regulates lymphocyte |
| | | | activation |
| IL5 | Interleukin 5 | Cytokines/ | Eosinophil stimulatory factor; stimulates late B |
| | | Chemokines/ | cell differentiation to secretion of Ig |
| | | Growth Factors | |
| IL6 | Interleukin 6 | cytokines- | Pro- and anti-inflammatory activity, TH2 |
| | (interferon, beta 2) | chemokines-growth | cytokine, regulates hematopoietic system and |
| | | factors | activation of innate response |
| IL8 | Interleukin 8 | cytokines- | Proinflammatory, major secondary |
| | | chemokines-growth | inflammatory mediator, cell adhesion, signal |
| | | factors | transduction, cell-cell signaling, angiogenesis, |
| | | | synthesized by a wide variety of cell types |
| INS | insulin | Insulin receptor | Decreases blood glucose concentration and |
| | | ligand | accelerates glycogen synthesis in the liver. Not |
| | | | as critical in NIDDM as in IDDM. |
| IRF5 | Interferon | Transcription | Regulates transcription of interferon genes |
| | regulatory factor 5 | Factor | through DNA sequence-specific binding. |
| | | | Diverse roles include virus-mediated activation |
| | | | of interferon, and modulation of cell growth, |
| | | | differentiation, apoptosis, and immune system |
| | | | activity. |
| IRS1 | insulin receptor | signal | Positive regulation of insulin action. This |
| | substrate 1 | transduction/transmembrane | protein is activated when insulin binds to |
| | | receptor | insulin receptor - binds 85-kDa subunit of PI 3- |
| | | protein | K. decreased in skeletal muscle of obese |
| | | | humans. |
| ITGAM | Integrin, alpha M; | Integrin | AKA; Complement receptor, type 3, alpha |
| | complement | | subunit; neutrophil adherence receptor; role in |
| | receptor | | adherence of neutrophils and monocytes to |
| | | | activate endothelium |
| IVL | Involucrin | structural protein- | Component of the keratinocyte cross linked |
| | | peripheral plasma | envelope; first appears in the cytosol becoming |
| | | membrane protein | cross linked to membrane proteins by |
| | | | transglutaminase |
| JUN | v-jun avian | transcription factor- | Proto-oncoprotein; component of transcription |
| | sarcoma virus 17 | DNA binding | factor AP-1 that interacts directly with target |
| | oncogene homolog | | DNA sequences to regulate gene expression |
| KAI1 | Kangai 1 | tumor suppressor | AKA SAR2, CD82, ST6: suppressor of |
| | | | metastatic ability of prostate cancer cells |
| K-ALPHA-1 | Alpha Tubulin, | microtubule | Major constituent of microtubules; binds 2 |
| | ubiquitous | peptide | molecules of GTP |
| KITLG | KIT ligand | Growth Factor | AKA Stem cell factor (SCF); mast cell growth |
| | | | factor, implicated in fibrosis/cirrhosis due to |
| | | | chronic liver inflammation |
| KLK2 | Kallikrein 2, | protease- | AKA hGK-1: Glandular kallikrein; expression |
| | prostatic | kallikrein | restricted mainly to the prostate. |
| KLK3 | Kallikrein 3 | protease- | AKA PSA: Kallikrein-like protease which |
| | | kallikrein | functions normally in liquefaction of seminal |
| | | | fluid. Elevated in prostate cancer. |
| KRT14 | Keratin 14 | structural protein- | Type I keratin; associates with keratin 5; |
| | | differentiation-cell | component of intermediate filaments; several |
| | | shape | autosomal dominant blistering skin disorders |
| | | | caused by gene defects |
| KRT16 | Keratin 16 | structural protein- | Type I keratin; component of intermediate |
| | | differentiation-cell | filaments; induced in skin conditions favoring |
| | | shape | enhanced proliferation or abnormal |
| | | | differentiation |
| KRT19 | Keratin 19 | structural protein- | AKA K19: Type I epidermal keratin; may form |
| | | differentiation | intermediate filaments |
| KRT5 | Keratin 5 | structural protein- | AKA EBS2: 58 kD Type II keratin co- |
| | | differentiation | expressed with keratin 14, a 50 kD Type I |
| | | | keratin, in stratified epithelium. KRT5 |
| | | | expression is a hallmark of mitotically active |
| | | | keratinocytes and is the primary structural |
| | | | component of the 10 nm intermediate filaments |
| | | | of the mitotic epidermal basal cells. |
| KRT8 | Keratin 8 | structural protein- | AKA K8, CK8: Type II keratin; coexpressed |
| | | differentiation | with Keratin 18; involved in intermediate |
| | | | filament formation |
| LGALS3 | Lectin, galactoside- | Liver Health | AKA galectin 3; Cell growth regulation |
| | binding, soluble, 3 | Indicator | |
| LGALS8 | Lectin, | cell adhesion- | AKA PCTA-1: binds to beta galactoside; |
| | Galactoside- | growth and | involved in biological processes such as cell |
| | binding, soluble 8 | differentiation | adhesion, cell growth regulation, inflammation, |
| | | | immunomodulation, apoptosis and metastasis |
| LBP | Lipopolysaccharide | Membrane protein | Acute phase protein; membrane protein that |
| | binding protein | | binds to Lipid a moity of bacterial LPS |
| MADD | MAP-kinase | co-receptor | Associates with TNFR1 through a death |
| | activating death | | domain-death domain interaction; Over |
| | domain | | expression of MADD activates the MAP kinase |
| | | | ERK2, and expression of the MADD death |
| | | | domain stimulates both the ERK2 and JNK1 |
| | | | MAP kinases and induces the phosphorylation |
| | | | of cytosolic phospholipase A2 |
| MAP3K14 | Mitogen-activated | kinase | Activator of NFKB1 |
| | protein kinase | | |
| | kinase kinase 14 | | |
| MAPK1 | mitogen-activated | Transferase | AKA ERK2; May promote entry into the cell |
| | protein kinase 1 | | cycle, growth factor responsive |
| MAPK8 | Mitogen | kinase-stress | aka JNK1; mitogen activated protein kinase |
| | Activated Protein | response-signal | regulates c-Jun in response to cell stress; UV |
| | Kinase 8 | transduction | irradiation of skin activates MAPK8 |
| MDM2 | Mdm2, | Oncogene/ | Inhibits p53- and p73-mediated cell cycle arrest |
| | transformed 3T3 | Transcription | and apoptosis by binding its transcriptional |
| | cell double minute | Factor | activation domain, resulting in tumorigenesis. |
| | 2, p53 binding | | Permits the nuclear export of p53 and targets it |
| | protein | | for proteasome-mediated proteolysis. |
| MIF | Macrophage | Cell signaling and | AKA; GIF; lymphokine, regulators macrophage |
| | migration | growth factor | functions through suppression of anti- |
| | inhibitory factor | | inflammatory effects of glucocorticoids |
| MMP1 | Matrix | Proteinase/ | aka Collagenase; cleaves collagens types I-III; |
| | Metalloproteinase 1 | Proteinase Inhibitor | plays a key role in remodeling occurring in both |
| | | | normal & diseased conditions; transcriptionally |
| | | | regulated by growth factors, hormones, |
| | | | cytokines & cellular transformation |
| MMP2 | Matrix | Proteinase/ | aka Gelatinase; cleaves collagens types IV, V, |
| | Metalloproteinase 2 | Proteinase Inhibitor | VII and gelatin type I; produced by normal skin |
| | | | fibroblasts; may play a role in regulation of |
| | | | vascularization & the inflammatory response |
| MMP3 | Matrix | Proteinase/ | AKA stromelysin; degrades fibronectin, laminin |
| | metalloproteinase 3 | Proteinase Inhibitor | and gelatin |
| MMP9 | Matrix | Proteinase/ | AKA gelatinase B; degrades extracellular |
| | metalloproteinase 9 | Proteinase Inhibitor | matrix molecules, secreted by IL-8-stimulated |
| | | | neutrophils |
| MP1 | Metalloprotease 1 | Proteinase/ | Member of the pitrilysin family. A |
| | | Proteinase Inhibitor | metalloendoprotease. Could play a broad role |
| | | | in general cellular regulation. |
| MRE11A | Meiotic | nuclease | Exonuclease involved in DNA double-strand |
| | recombination (S. cerevisiae) | | breaks repair |
| | 11 | | |
| | homolog A | | |
| MYC | V-myc avian | transcription factor- | Transcription factor that promotes cell |
| | myelocytomatosis | oncogene | proliferation and transformation by activating |
| | viral oncogene | | growth-promoting genes; may also repress gene |
| | homolog | | expression |
| N33 | Putative prostate | Tumor Suppressor | Integral membrane protein. Associated with |
| | cancer tumor | | homozygous deletion in metastatic prostate |
| | suppressor | | cancer. |
| NFKB1 | Nuclear factor of | Transcription | p105 is the precursor of the p50 subunit of the |
| | kappa light | Factor | nuclear factor NFKB, which binds to the kappa- |
| | polypeptide gene | | b consensus sequence located in the enhancer |
| | enhancer in B- | | region of genes involved in immune response |
| | cells 1 (p105) | | and acute phase reactions; the precursor does |
| | | | not bind DNA itself |
| NFKBIB | Nuclear factor of | Transcription | Inhibits/regulates NFKB complex activity by |
| | kappa light | Regulator | trapping NFKB in the cytoplasm. |
| | polypeptide gene | | Phosphorylated serine residues mark the |
| | enhancer in B- | | NFKBIB protein for destruction thereby |
| | cells inhibitor, | | allowing activation of the NFKB complex. |
| | beta | | |
| NOS1 | Mitric oxide | Enzyme/redox | Synthesizes nitric oxide from L-arginine and |
| | synthase 1 | | molecular oxygen, regulates skeletal muscle |
| | (neuronal) | | vasoconstriction, body fluid homeostasis, |
| | | | neuroendocrine physiology, smooth muscle |
| | | | motility, and sexual function |
| NOS2A | Nitric oxide | Enzyme/Redox | AKA iNOS; produces NO which is |
| | synthase 2A | | bacteriocidal/tumoricidal |
| NOS3 | Nitric oxide | Enzyme/redox | Enzyme found in endothelial cells mediating |
| | synthase 3 | | smooth muscle relation; promotes clotting |
| | | | through the activation of platelets. |
| NR1I2 | Nuclear receptor | transcription | aka PAR2; Member of nuclear hormone |
| | subfamily 1 | activation factor- | receptor family of ligand-activated transcription |
| | | signal transduction- | factors; activates transcription of cytochrome P- |
| | | xenobiotic | 450 genes |
| | | metabolism | |
| NR1I3 | Nuclear receptor | Metabolism | AKA Constitutive androstane receptor beta |
| | subfamily 1, | Receptor/Transcription | (CAR); heterodimer with retinoid X receptor |
| | group I, family 3 | Factor | forms nuclear transcription factor; mediates |
| | | | P450 induction by Phenobarbital-like inducers. |
| NRP1 | Neuropilin 1 | cell adhesion | AKA NRP, VEGF165R: A novel VEGF |
| | | | receptor that modulates VEGF binding to KDR |
| | | | (VEGF receptor) and subsequent bioactivity |
| | | | and therefore may regulate VEGF-induced |
| | | | angiogenesis; calcium-independent cell |
| | | | adhesion molecule that function during the |
| | | | formation of certain neuronal circuits |
| ORM1 | Orosomucoid 1 | Liver Health | AKA alpha 1 acid glycoprotein (AGP), acute |
| | | Indicator | phase inflammation protein |
| OXCT | 3-oxoacid CoA | Transferase | OXCT catalyzes the reversible transfer of |
| | transferase | | coenzyme A from succinyl-CoA to acetoacetate |
| | | | as the first step of ketolysis (ketone body |
| | | | utilization) in extrahepatic tissues. |
| PART1 | Prostate | | Exhibits increased expression in LNCaP cells |
| | androgen- | | upon exposure to androgens |
| | regulated | | |
| | transcript 1 | | |
| PCA3 | Prostate cancer | | AKA DD3: prostate specific; highly expressed |
| | antigen 3 | | in prostate tumors |
| PCANAP7 | Prostate cancer | | AKA IPCA7: unknown function; co-expressed |
| | associated protein 7 | | with known prostate cancer genes |
| PCK1 | phosphoenolpyruvate | rate-limiting | Rate limiting enzyme for gluconeogenesis - |
| | carboxykinase 1 | gluconeogenic | plays a key role in the regulation of hepatic |
| | | enzyme | glucose output by insulin and glucagon. Over |
| | | | expression in the liver results in increased |
| | | | hepatic glucose production and hepatic insulin |
| | | | resistance to glycogen synthe |
| PCNA | Proliferating Cell | DNA binding-DNA | Required for both DNA replication & repair; |
| | Nuclear Antigen | replication-DNA | processivity factor for DNA polymerases delta |
| | | repair-cell | and epsilon |
| | | proliferation | |
| PCTK1 | PCTAIRE protein | | Belongs to the SER/THR family of protein |
| | kinase 1 | | kinases; CDC2/CDKX subfamily. May play a |
| | | | role in signal transduction cascades in |
| | | | terminally differentiated cells. |
| PDCD8 | Programmed Cell | enzyme, reductase | The principal mitochondrial factor causing |
| | Death 8 | | nuclear apoptosis. Independent of caspase |
| | (apoptosis- | | apoptosis. |
| | inducing factor) | | |
| PDEF | Prostate | transcription factor | Acts as an androgen-independent transcriptional |
| | epithelium | | activator of the PSA promoter; directly interacts |
| | specific Ets | | with the DNA binding domain of androgen |
| | transcription | | receptor and enhances androgen-mediated |
| | factor | | activation of the PSA promoter |
| PF4 | Platelet Factor 4 | Chemokine | PF4 is released during platelet aggregation and |
| | (SCYB4) | | is chemotactic for neutrophils and monocytes. |
| | | | PF4's major physiologic role appears to be |
| | | | neutralization of heparin-like molecules on the |
| | | | endothelial surface of blood vessels, thereby |
| | | | inhibiting local antithrombin III activity and |
| | | | promoting coagulation. |
| PI3 | Proteinase | proteinase | aka SKALP; Proteinase inhibitor found in |
| | inhibitor 3 skin | inhibitor-protein | epidermis of several inflammatory skin |
| | derived | binding- | diseases; it's expression can be used as a marker |
| | | extracellular matrix | of skin irritancy |
| PIK3R1 | phosphoinositide- | regulatory enzyme | Positive regulation of insulin action. Docks in |
| | 3-kinase, | | IRS proteins and Gab1 - activity is required for |
| | regulatory | | insulin stimulated translocation of glucose |
| | subunit, | | transporters to the plasma membrane and |
| | polypeptide 1 | | activation of glucose uptake. |
| | (p85 alpha) | | |
| PLA2G7 | Phospholipase A2, | Enzyme/Redox | Platelet activating factor |
| | group VII (platelet | | |
| | activating factor | | |
| | acetylhydrolase, | | |
| | plasma) | | |
| PLAT | Plasminogen | Protease | AKA TPA; Converts plasminogin to plasmin; |
| | activator, tissue | | involved in fibrinolysis and cell migration |
| PLAU | Plasminogen | Proteinase/ | AKA uPA; cleaves plasminogen to plasmin (a |
| | activator, | Proteinase Inhibitor | protease responsible for nonspecific |
| | urokinase | | extracellular matrix degradation) |
| PNKP | Polynucleotide | phosphatase | Catalyzes the 5-prime phosphorylation of |
| | kinase 3′- | | nucleic acids and can have associated 3-prime |
| | phosphatase | | phosphatase activity, predictive of an important |
| | | | function in DNA repair following ionizing |
| | | | radiation or oxidative damage |
| POV1 | Prostate cancer | | RNA expressed selectively in prostate tumor |
| | overexpressed | | samples |
| | gene 1 | | |
| PPARA | Peroxisome | Metabolism | Binds peroxisomal proliferators (ie fatty acids, |
| | proliferator | Receptor | hypolipidemic drugs) & controls pathway for |
| | activated receptor □ | | beta-oxidation of fatty acids |
| PPARG | peroxisome | transcription | The primary pharmacological target for the |
| | proliferator- | factor/Ligand- | treatment of insulin resistance in NIDDM. |
| | activated receptor, | dependent nuclear | Involved in glucose and lipid metabolism in |
| | gamma | receptor | skeletal muscle. |
| PRKCB1 | protein kinase C, | protein kinase | Negative regulation of insulin action. Activated |
| | beta 1 | C/protein | by hyperglycemia - increases phosphorylation |
| | | phosphorylation | of IRS-1 and reduces insulin receptor kinase |
| | | | activity. Increased PKC activation may lead to |
| | | | oxidative stress causing over expression of |
| | | | TGF-beta and fibronectin |
| PSCA | Prostate stem cell | antigen | Prostate-specific cell surface antigen expressed |
| | antigen | | strongly by both androgen-dependent and - |
| | | | independent tumors |
| PTEN | Phosphatase and | tumor suppressor | Tumor suppressor that modulates G1 cell cycle |
| | tensin homolog | | progression through negatively regulating the |
| | (mutated in | | PI3-kinase/Akt signaling pathway; one critical |
| | multiple advanced | | target of this signaling process is the cyclin- |
| | cancers 1) | | dependent kinase inhibitor p27 (CDKN1B). |
| PTGIS | Prostaglandin I2 | Isomerase | AKA PGIS; PTGI; CYP8; CYP8A1; Converts |
| | (prostacyclin) | | prostaglandin h2 to prostacyclin (vasodilator); |
| | synthase | | cytochrome P450 family; imbalance of |
| | | | prostacyclin may contribute to myocardial |
| | | | infarction, stroke, atherosclerosis |
| PTGS2 | Prostaglandin- | Enzyme/Redox | AKA COX2; Proinflammatory, member of |
| | endoperoxide | | arachidonic acid to prostanoid conversion |
| | synthase 2 | | pathway; induced by proinflammatory |
| | | | cytokines |
| PTPRC | protein tyrosine | Cell Marker | AKA CD45; mediates T-cell activation |
| | phosphatase, | | |
| | receptor type, C | | |
| PTX3 | pentaxin-related | | AKA TSG-14; Pentaxin 3; Similar to the |
| | gene, rapidly | | pentaxin subclass of inflammatory acute-phase |
| | induced by IL-1 | | proteins; novel marker of inflammatory |
| | beta | | reactions |
| RAD52 | RAD52 (S. cerevisiae) | DNA binding | Involved in DNA double-stranded break repair |
| | homolog | proteinsor | and meiotic/mitotic recombination |
| RB1 | Retinoblastoma 1 | tumor suppressor | Regulator of cell growth; interacts with E2F- |
| | (including | | like transcription factor; a nuclear |
| | osteosarcoma) | | phosphoprotein with DNA binding activity; |
| | | | interacts with histone deacetylase to repress |
| | | | transcription |
| S100A7 | S100 calcium- | calcium binding- | Member of S100 family of calcium binding |
| | binding protein 7 | epidermal | proteins; localized in the cytoplasm &/or |
| | | differentiation | nucleus of a wide range of cells; involved in the |
| | | | regulation of cell cycle progression & |
| | | | differentiation; markedly overexpressed in skin |
| | | | lesions of psoriatic patients |
| SCYA2 | Small inducible | Cytokine/Chemokine | AKA Monocyte chemotactic protein 1 (MCP1); |
| | cytokine A2 | | recruits monocytes to areas of injury and |
| | | | infection, unregulated in liver inflammation |
| SCYA3 | small inducible | Chemokine | A “monokine” involved in the acute |
| | cytokine A3 | | inflammatory state through the recruitment and |
| | (MIP1a) | | activation of polymorphonuclear leukocytes. A |
| | | | major HIV-suppressive factor produced by |
| | | | CD8-positive T cells. |
| SCYA5 | small inducible | Chemokine | Binds to CCR1, CCR3, and CCR5 and is a |
| | cytokine A5 | | Chemoattractant for blood monocytes, memory |
| | (RANTES) | | t helper cells and eosinophils. A major HIV- |
| | | | suppressive factor produced by CD8-positive T |
| | | | cells. |
| SCYB10 | small inducible | Chemokine | A CXC subfamily chemokine. Binding of |
| | cytokine | | SCYB10 to receptor CXCR3/GPR9 results in |
| | subfamily B (Cys- | | stimulation of monocytes, natural killer and T- |
| | X-Cys), member | | cell migration, and modulation of adhesion |
| | 10 | | molecule expression. SCYB10 is Induced by |
| | | | IFNg and may be a key mediator in IFNg |
| | | | response. |
| SDF1 | stromal cell- | Chemokine | Belongs to the CXC subfamily of the intercrine |
| | derived factor 1 | | family, which activates leukocytes. SDF1 is the |
| | | | primary ligand for CXCR4, a coreceptor with |
| | | | CD4 for human immunodeficiency virus type 1 |
| | | | (HIV-1). SDF1 is a highly efficacious |
| | | | lymphocyte Chemoattractant. |
| SELE | selectin E | Cell Adhesion | AKA ELAM; Expressed by cytokine-stimulated |
| | (endothelial | | endothelial cells; mediates adhesion of |
| | adhesion molecule | | neutrophils to the vascular lining |
| | 1) | | |
| SERPINB5 | Serine proteinase | Proteinase/ | Protease Inhibitor; Tumor suppressor, |
| | inhibitor, clade B, | Proteinase Inhibitor/ | especially for metastasis. Inhibits tumor |
| | member 5 | Tumor Suppressor | invasion by inhibiting cell motility. |
| SERPINE1 | Serine (or | Proteinase/ | Plasminogen activator inhibitor-1/PAI-1 |
| | cysteine) protease | Proteinase Inhibitor | |
| | inhibitor, clade B | | |
| | (ovalbumin), | | |
| | member 1 | | |
| SFTPD | Surfactant, | Extracellular | AKA; PSPD; mannose-binding protein |
| | pulmonary | Lipoprotein | associated with pulmonary surfactant |
| | associated protein D | | |
| SLC2A2 | solute carrier | glucose transporter | Glucose transporters expressed uniquely in b- |
| | family 2 | | cells and liver. Transport glucose into the b- |
| | (facilitated | | cell. Typically under expressed in pancreatic |
| | glucose | | islet cells of individuals with NIDDM. |
| | transporter), | | |
| | member 2 | | |
| SLC2A4 | solute carrier | glucose transporter | Glucose transporter protein that is final |
| | family 2 | | mediator in insulin-stimulated glucose uptake |
| | (facilitated | | (rate limiting for glucose uptake). Under |
| | glucose | | expression not important, but over expression in |
| | transporter), | | muscle and adipose tissue consistently shown to |
| | member 4 | | increase glucose transport. |
| SMAC | Second | mitochondrial | Promotes caspase activation in cytochrome c/ |
| | mitochondria- | peptide | APAF-1/caspase 9 pathway of apoptosis |
| | derived activator | | |
| | of caspase | | |
| SOD2 | superoxide | Oxidoreductase | Enzyme that scavenges and destroys free |
| | dismutase 2, | | radicals within mitochondria |
| | mitochondrial | | |
| SRP19 | Signal recognition | | Responsible for signal-recognition-particle |
| | particle 19 kD | | assembly. SRP mediates the targeting of |
| | | | proteins to the endoplasmic reticulum. |
| STAT1 | Signal transducer | DNA-Binding | Binds to the IFN-Stimulated Response Element |
| | and activator of | Protein | (ISRE) and to the GAS element; specifically |
| | transcription 1, | | required for interferon signaling. STAT1 can |
| | 91 kD | | be activated by IFN-alpha, IFN-gamma, EGF, |
| | | | PDGF and IL6. BRCA1-regulated genes |
| | | | overexpressed in breast tumorigenesis included |
| | | | STAT1 and JAK1. |
| STAT3 | Signal | transcription factor | AKA APRF: Transcription factor for acute |
| | transduction and | | phase response genes; rapidly activated in |
| | activator of | | response to certain cytokines and growth |
| | transcription 3 | | factors; binds to IL6 response elements |
| TACI | Tumor necrosis | cytokines- | T cell activating factor and calcium cyclophilin |
| | factor receptor | chemokines-growth | modulator |
| | superfamily, | factors | |
| | member 13b | | |
| TEK | tyrosine kinase, | Transferase | AKA TIE2, VMCM; Receptor for angiopoietin- |
| | endothelial | Receptor | 1; may regulate endothelial cell proliferation |
| | | | and differentiation; involved in vascular |
| | | | morphogenesis; TEK defects are associated |
| | | | with venous malformations |
| TERT | Telomerase | transcriptase | Ribonucleoprotein which in vitro recognizes a |
| | reverse | | single-stranded G-rich telomere primer and |
| | transcriptase | | adds multiple telomeric repeats to its 3-prime |
| | | | end by using an RNA template |
| TGFA | Transforming | Transferase/ | Proinflammatory cytokine that is the primary |
| | Growth Factor, | Signal | mediator of immune response and regulation, |
| | Alpha | Transduction | Associated with TH₁ responses, mediates host |
| | | | response to bacterial stimuli, regulates cell |
| | | | growth & differentiation; Negative regulation of |
| | | | insulin action |
| TGFB1 | Transforming | cytokines- | Pro- and anti-inflammatory activity, anti- |
| | growth factor, | chemokines-growth | apoptotic; cell-cell signaling, can either inhibit |
| | beta 1 | factors | or stimulate cell growth |
| TGFB3 | Transforming | Cell Signaling | Transmits signals through transmembrane |
| | growth factor, | | serine/threonine kinases. Increased expression |
| | beta 3 | | of TGFB3 may contribute to the growth of |
| | | | tumors. |
| TGFBR2 | Transforming | Membrane protein | AKA: TGFR2; membrane protein involved in |
| | growth factor, | | cell signaling and activation, ser/thr protease; |
| | beta receptor II | | binds to DAXX. |
| TIMP1 | tissue inhibitor of | Proteinase/ | Irreversibly binds and inhibits |
| | metalloproteinase 1 | Proteinase Inhibitor | metalloproteinases, such as collagenase |
| TLR2 | toll-like receptor 2 | cell signaling and | mediator of petidoglycan and lipotechoic acid |
| | | activation | induced signaling |
| TLR4 | toll-like receptor 4 | cell signaling and | mediator of LPS induced signaling |
| | | activation | |
| TLX3 | T-cell leukemia, | Transcription | Member of the homeodomain family of DNA |
| | homeobox 3 | Factor | binding proteins. May be activated in T-ALL |
| | | | leukomogenesis. |
| TNF | tumor necrosis | cytokine/tumor | Negative regulation of insulin action. Produced |
| | factor | necrosis factor | in excess by adipose tissue of obese individuals - |
| | | receptor ligand | increases IRS-1 phosphorylation and |
| | | | decreases insulin receptor kinase activity. |
| TNFA | Tumor Necrosis | Cytokines/ | Pro-inflammatory; TH₁ cytokine; Mediates host |
| | Factor, Alpha | Chemokines/ | response to bacterial stimulus; Regulates cell |
| | | Growth factors | growth & differentiation |
| TNFRSF11A | Tumor necrosis | receptor | Activates NFKB1; Important regulator of |
| | factor receptor | | interactions between T cells and dendritic cells |
| | superfamily, | | |
| | member 11a, | | |
| | activator of NFKB | | |
| TNFRSF12 | Tumor necrosis | receptor | Induces apoptosis and activates NF-kappaB; |
| | factor receptor | | contains a cytoplasmic death domain and |
| | superfamily, | | transmembrane domains |
| | member 12 | | |
| | (translocating | | |
| | chain-association | | |
| | membrane | | |
| | protein) | | |
| TNFSF13B | Tumor necrosis | cytokines- | B cell activating factor, TNF family |
| | factor (ligand) | chemokines-growth | |
| | superfamily, | factors | |
| | member 13b | | |
| TNFSF5 | Tumor necrosis | cytokines- | Ligand for CD40; expressed on the surface of T |
| | factor (ligand) | chemokines-growth | cells. It regulates B cell function by engaging |
| | superfamily, | factors | CD40 on the B cell surface. |
| | member 5 | | |
| TNFSF6 | Tumor necrosis | cytokines- | AKA FasL; Ligand for FAS antigen; transduces |
| | factor (ligand) | chemokines-growth | apoptotic signals into cells |
| | superfamily, | factors | |
| | member 6 | | |
| TOSO | Regulator of Fas- | receptor | Potent inhibitor of Fas induced apoptosis; |
| | induced apoptosis | | expression of TOSO, like that of FAS and |
| | | | FASL, increases after T-cell activation, |
| | | | followed by a decline and susceptibility to |
| | | | apoptosis; hematopoietic cells expressing |
| | | | TOSO resist anti-FAS-, FADD-, and TNF- |
| | | | induced apoptosis without increasing expression |
| | | | of the inhibitors of apoptosis BCL2 and |
| | | | BCLXL; cells expressing TOSO and activated |
| | | | by FAS have reduced CASP8 and increased |
| | | | CFLAR expression, which inhibits CASP8 |
| | | | processing |
| TP53 | Tumor protein 53 | DNA binding | AKA P53: Activates expression of genes that |
| | | protein-cell cycle- | inhibit tumor growth and/or invasion; involved |
| | | tumor suppressor | in cell cycle regulation (required for growth |
| | | | arrest at G1); inhibits cell growth through |
| | | | activation of cell-cycle arrest and apoptosis |
| TRADD | TNFRSF1A- | co-receptor | Over expression of TRADD leads to 2 major |
| | associated via | | TNF-induced responses, apoptosis and |
| | death domain | | activation of NF-kappa-B |
| TRAF1 | TNF receptor- | co-receptor | Interact with cytoplasmic domain of TNFR2 |
| | associated factor 1 | | |
| TRAF2 | TNF receptor- | co-receptor | Interact with cytoplasmic domain of TNFR2 |
| | associated factor 2 | | |
| TREM1 | Triggering | cell signaling and | Member of the lg superfamily; receptor |
| | receptor expressed | activation | exclusively expressed on myeloid cells. |
| | on myeloid cells 1 | | TREM1 mediates activation of neutrophils and |
| | | | monocytes and may have a predominant role in |
| | | | inflammatory responses. |
| UCP2 | Uncoupling | Liver Health | Decouples oxidative phosphorylation from ATP |
| | protein 2 | Indicator | synthesis, linked to diabetes, obesity |
| UGT | UDP- | Metabolism | Catalyzes glucuronide conjugation to metabolic |
| | Glucuronosyltransferase | Enzyme | substrates, primer-probe set nonspecific for all |
| | | | members of UGT1 family |
| VCAM1 | vascular cell | Cell Adhesion/ | AKA L1CAM; CD106; INCAM-100; Cell |
| | adhesion molecule 1 | Matrix Protein | surface adhesion molecule specific for blood |
| | | | leukocytes and some tumor cells; mediates |
| | | | signal transduction; may be linked to the |
| | | | development of atherosclerosis, and rheumatoid |
| | | | arthritis |
| VDAC1 | Voltage- | membrane protein | Functions as a voltage-gated pore of the outer |
| | dependent anion | | mitochondrial membrane; proapoptotic proteins |
| | channel 1 | | BAX and BAK accelerate the opening of |
| | | | VDAC allowing cytochrome c to enter, whereas |
| | | | the antiapoptotic protein BCL2L1 closes VDAC |
| | | | by binding directly to it |
| VEGF | vascular | cytokines- | VPF: Induces vascular permeability, endothelial |
| | endothelial | chemokines-growth | cell proliferation, and angiogenesis. Produced |
| | growth factor | factors | by monocytes |
| VWF | Von Willebrand | Coagulation Factor | Multimeric plasma glycoprotein active in the |
| | factor | | blood coagulation system as an antihemophilic |
| | | | factor (VIIIC) carrier and platelet-vessel wall |
| | | | mediator. Secreted by endothelial cells. |
| XRCC5 | X-ray repair | helicase | Functions together with the DNA ligase IV- |
| | complementing | | XRCC4 complex in the repair of DNA double- |
| | defective repair in | | strand breaks |
| | Chinese hamster | | |
| | cells 5 | | |

What is claimed is:

1.-195. (canceled)

196. A method for evaluating infectious disease or inflammatory conditions related to infectious disease of a target population of cells affected by a first agent in relation to the infectious disease or inflammatory conditions related to infectious disease of the target population of cells affected by a second agent, based on a first sample from the target population cells to which the first agent has been administered and a second sample from the target population of cells to which the second agent has been administered, the samples providing a source of RNAs, the method comprising:
deriving from the first sample a first profile data set and from the second sample a second profile data set, the first and second profile data sets each including a plurality of members, each member being a quantitative measure of the amount of a distinct RNA constituent in a panel of constituents selected so that measurement of the constituents enables evaluation of the infectious disease or inflammatory conditions related to infectious disease affected by the first agent in relation to the second agent, wherein such measure for each constituent is obtained under measurement conditions that are substantially repeatable; and
producing a first calibrated profile data set and a second calibrated profile data set for the panel, wherein (i) each member of the first calibrated profile data set is a function of a corresponding member of the first profile data set and a corresponding member of a baseline profile data set for the panel, and (ii) each member of the second calibrated profile data set is a function of a corresponding member of the second profile data set and a corresponding member of the baseline profile data set, wherein each member of the baseline data set is a normative measure, determined with respect to a relevant set of subjects, of the amount of one of the constituents in the panel, and wherein the baseline profile data set is related to the infectious disease or inflammatory conditions related to infectious disease to be evaluated, the first and second calibrated profile data sets being a comparison between the first profile data set and the baseline profile set and a comparison between the second profile data set and the baseline profile data set, thereby providing an evaluation of the infectious disease or inflammatory conditions related to infectious disease of the target population of cells affected by the first agent in relation to the infectious disease or inflammatory conditions related to infectious disease of the target population of cells affected by the second agent.

197. A method according to claim 196, wherein the target population of cells has presumptive signs of a systemic infection including at least one of: elevated white blood cell count, elevated temperature, elevated heart rate, and elevated or reduced blood pressure, relative to medical standards.

198. A method according to claim 196, wherein the target population of cells has presumptive signs of a systemic infection that are related to inflammatory conditions arising from at least one of: blunt or penetrating trauma, surgery, endocarditis, urinary tract infection, pneumonia, or dental or gynecological examinations or treatments.

199. A method according to claim 196, wherein the first agent is a first drug and the second agent is a second drug.

200. A method according to claim 196, wherein the first agent is a drug and the second agent is a complex mixture.

201. A method according to claim 196, wherein the first agent is a drug and the second agent is a nutriceutical.

202. A method according to claim 196, wherein the quantitative measure is determined by amplification, and the measurement conditions are such that efficiencies of amplification for all constituents differ by less than approximately 2 percent.

203. A method according to claim 196, wherein the quantitative measure is determined by amplification, and the measurement conditions are such that efficiencies of amplification for all constituents differ by less than approximately 1 percent.

204. A method according to claim 196, wherein the measurement conditions that are substantially repeatable are within a degree of repeatability of better than five percent.

205. A method according to claim 196, wherein the measurement conditions that are substantially repeatable are within a degree of repeatability of better than three percent.

206. A method according to claim 196, wherein the infectious disease or inflammatory conditions related to infectious disease being evaluated is with respect to a localized tissue of the subject and the first sample is derived from tissue or fluid of a type distinct from that of the localized tissue.

207. A method according to claim 196, wherein the infectious disease or inflammatory conditions related to infectious disease is a microbial infection.

208. A method according to claim 196, wherein the infectious disease or inflammatory conditions related to infectious disease is a bacterial infection.

209. A method according to claim 196, wherein the infectious disease or inflammatory conditions related to infectious disease is a eukaryotic parasitic infection.

210. A method according to claim 196, wherein the infectious disease or inflammatory conditions related to infectious disease is a viral infection.

211. A method according to claim 196, wherein the infectious disease or inflammatory conditions related to infectious disease is a fungal infection.

212. A method according to claim 196, wherein the infectious disease or inflammatory conditions related to infectious disease is systemic inflammatory response syndrome (SIRS).

213. A method according to claim 196, wherein the infectious disease or inflammatory conditions related to infectious disease is bacteremia, viremia, or fungemia.

214. A method according to claim 196, wherein the infectious disease or inflammatory conditions related to infectious disease is septicemia due to any class of microbe.

215.-216. (canceled)

217. A method according to claim 196, wherein the baseline profile data set is derived from one or more other samples from the same subject taken under circumstances different from those of the first sample.

218. A method according to claim 196, wherein the baseline profile data set is derived from one or more other samples from the same subject taken under circumstances different from those of the second sample.

219.-262. (canceled)
